(19) United States
(12) Patent Application Publication
(10) Pub. No.: US 2025/0235493 A1
(43) Pub. Date: Jul. 24, 2025

(54) Recombinant Viral Genomes and Related Compositions and Methods

(71) Applicant: The Board of Trustees of the Leland Stanford Junior University, Stanford, CA (US)

(72) Inventors: Michael Z. Lin, Stanford, CA (US); Xinzhi Zou, Redwood City, CA (US); Hokyung Chung, San Diego, CA (US)

(21) Appl. No.: 18/705,463

(22) PCT Filed: Oct. 27, 2022
(86) PCT No.: PCT/US2022/078795

§ 371 Date: Apr. 26, 2024

Related U.S. Application Data
(60) Provisional application No. 63/272,515, filed on Oct. 27, 2021.

(51) Int. Cl. A61K 35/766; A61K 35/761; C12N 7/00

(52) U.S. Cl. CPC A61K 35/766; A61K 35/761; C12N 7/00; C12N 2710/10021; C12N 2710/10032; C12N 2710/10062; C12N 2760/20221; C12N 2760/20232; C12N 2760/20262; C12N 2820/007

(57) ABSTRACT

Provided are recombinant viral genomes. The viral genomes find use in a variety of contexts including for the production of viruses effective in inducing death of cells exhibiting a signal specific to a disease or disorder in a subject, where the viral life cycle is dependent upon the signal specific to the disease or disorder. According to some embodiments, the viral genomes comprise one or more transcription units comprising a sequence encoding a first fusion protein comprising a protease and a first binding domain that binds to a first target, and a sequence encoding a second fusion protein comprising a membrane-targeting signal, a second binding domain that binds to a second target, a cleavable substrate for the protease, and either a transcriptional effector protein or viral protein encoded by a viral gene deleted in the viral genome. Also provided are related cells, virions, pharmaceutical compositions and methods of use.

### CROSS-REFERENCE TO RELATED APPLICATIONS

This application claims the benefit of U.S. Provisional Patent Application No. 63/272,515, filed Oct. 27, 2021, which application is incorporated herein by reference in its entirety.

### INTRODUCTION

Cancer continues to kill millions each year worldwide and is a major determinant of life expectancy in developed countries. There are thus high levels of interest in developing cancer therapies with improved efficacy. The possibility of using viruses as anti-cancer agents has been widely tested and, in one case, has been proven in randomized controlled clinical trials. A large variety of viruses have been tested for their ability to replicate in and kill cancer cells in vitro and in vivo. Research in recent years has indicated that anti-cancer effects in animal models derives both from direct killing of cancer cells by the virus, and from the development of immune responses against tumor antigens following viral infection. However, currently described virotherapies for cancer demonstrate unsatisfactory specificity and efficacy.

For example, the single approved cancer virotherapy, T-VEC or talimogene laherparepvec, is indicated for melanoma, but requires intratumoral injection to prevent off-target effects. T-VEC is a herpes simplex virus 1 (HSV1) derivative with deletions of the ICP34.5 genes to reduce virulence and addition of a GM-CSF gene to provide immunostimulation. Its primary mechanism of action is believed to be via eliciting of an immune response against the tumor, as preclinical trials showed the generation of an immune response against tumor cells and poor activity without GM-CSF (1). However, melanoma is especially immunogenic, and T-VEC has yet to demonstrate efficacy vs. standard of care in other indications. For example, it recently failed a clinical trial in squamous cell carcinoma (2). How TVEC can be made more efficacious without causing adverse effects is unclear; existing ideas include picking more virulent strains and adding an immunoescape gene, but these manipulations are likely to enhance replication in normal cells as well.

Other examples of poor selectivity and efficacy comes from a virus under investigation as a cancer therapy, vesicular stomatitis virus (VSV). VSV with mutations of position 51 in the matrix (M) protein were believed to preferentially replicate in cells that lack innate antiviral immunity components, which includes many cancer cells (3), but it also replicates in some non-tumorigenic cells lines as well (4), and replicates more poorly than wild-type VSV across all cell lines tested (4, 5). To bolster protection to normal cells when used as a cancer virotherapy, VSV was engineered to express IFNβ, which activates the innate antiviral immune response. However IFNβ addition also attenuates replication in some cancer cells (6), and even then VSV-IFNβ shows hepatotoxicity and lymphopenia in rats after systemic injection as a result of nonspecific viral entry and replication (7).

Additional examples of poor selectivity and efficacy comes from yet another class of viruses under investigation as cancer therapies, adenoviruses. Adenoviruses with E1B deletions replicate preferentially in some cancer cells compared to non-cancer cells, but replication is attenuated by orders of magnitude relative to wild-type adenovirus (8). E1B-deleted adenoviruses failed to advance beyond Phase 2 trials due to poor efficacy and a high rate of adverse events (9).

As illustrated by the examples above, a method to regulate virus replication so it only occurs in cancer cells would be useful for improving virotherapies. Here, it would be useful to make viral replication dependent on biochemical signals that are present only in cancer cells. Cancer cells differ fundamentally from normal cells in constitutively activating signaling pathways promoting cell growth and proliferation (10), through the mutation or overexpression of cancer-causing genes, known as oncogenes. For example, a substantial fraction of colon, stomach, lung, breast, and brain cancers feature constitutive (always-on) activation of ErbB proteins, a family of transmembrane receptor tyrosine kinases that include epidermal growth factor receptor (EGFR) and human EGF receptor 2 (HER2). Mutation or overexpression of an ErbB gene causes constitutive autophosphorylation on tyrosine residues of the encoded ErbB protein. This hyperactive protein, which can be termed an oncoprotein, then drives activation of intracellular signals to promote non-physiological survival, growth, and proliferation of the cell (11). Indeed, treatments for cancers harboring constitutive ErbB activity (ErbB-positive cancers) have thus been developed based on inhibiting the intrinsic tyrosine kinase enzymatic activity with small-molecule drugs (11), or mediating an immune reaction against cells with high ErbB expression using ErbB-specific antibodies (11) or T-cell receptors (12). However, as these treatments target all ErbB proteins and not just the mutated ones, they are limited by toxicity to normal cells.

### SUMMARY

Provided are recombinant viral genomes. The viral genomes find use in a variety of contexts including for the production of viruses effective in inducing death of cells exhibiting a signal specific to a disease or disorder in a subject, where the viral life cycle is dependent upon the signal specific to the disease or disorder. According to some embodiments, the viral genomes comprise one or more transcription units comprising a sequence encoding a first fusion protein comprising a protease and a first binding domain that binds to a first target, and a sequence encoding a second fusion protein comprising a membrane-targeting signal, a second binding domain that binds to a second target, a cleavable substrate for the protease, and either a transcriptional effector protein or viral protein encoded by a viral gene deleted in the viral genome. Also provided are related cells, virions, pharmaceutical compositions and methods of use.

In an embodiment, a recombinant viral genome comprises a first transcription unit, comprising a first sequence encoding a first fusion protein. The first fusion protein comprises a protease and a first binding domain that binds to a first target. The first transcription unit further comprises a second sequence encoding a second fusion protein comprising, in N-terminal to C-terminal order or in C-terminal to N-terminal order, a membrane-targeting signal, a second binding domain that binds to a second target, a substrate cleavable by the protease, and a transcriptional effector protein. A second transcription unit comprises a third sequence encoding one or more viral replication proteins, wherein the second transcription unit is operably linked to a promoter activatable by the transcriptional effector protein.

In some embodiments, the recombinant viral genome comprises a modified genome of a virus. In some embodiments, the virus is an oncolytic virus. In some embodiments, the promoter is not activatable by the transcriptional effector protein until the transcriptional effector protein is cleaved from the second fusion protein. In some embodiments, the promoter is activatable to a lesser extent by the second fusion protein compared to the transcriptional effector protein when cleaved from the second fusion protein. In some embodiments, the transcriptional effector protein comprises a transcription factor. In some embodiments, the one or more viral replication proteins encoded by the second transcription unit is required for replication of the virus in cells or efficient replication of the virus in cells. In some embodiments, replication of the virus in cells is dependent on a signal specific to cancer cells or a signal in cancer cells that is higher than the signal in non-cancer cells. In some embodiments, replication of the virus in cells is not dependent on the presence of an endogenous transcription factor. In some embodiments, the endogenous transcription factor is an endogenous cancer-selective transcription factor. In some embodiments, the recombinant viral genome further comprises a third transcription unit encoding an immunostimulatory molecule, optionally wherein the immunostimulatory molecule is granulocyte/macrophage colony stimulating factor (GM-CSF). In some embodiments, the virus is a double-stranded DNA virus. In some embodiments, the double-stranded DNA virus is an adenovirus. In some embodiments, the one or more viral replication proteins comprise one or more E1 gene products. In some embodiments, the transcriptional effector protein comprises a GAL4-VP16 transcription factor. In some embodiments, the promoter activatable by the transcriptional effector protein is a GAL4 UAS promoter.

In an embodiment, a recombinant viral genome comprises a deficiency in at least one native viral gene and one or more transcription units. The one or more transcription units comprises a sequence encoding a first fusion protein comprising a protease and a first binding domain that binds to a first target, and a sequence encoding a second fusion protein comprising, in N-terminal to C-terminal order or in C-terminal to N-terminal order, a membrane-targeting signal, a second binding domain that binds to a second target, a substrate cleavable by the protease, and the viral protein encoded by the deleted gene.

In some embodiments, the recombinant viral genome comprises a modified genome of a virus. In some embodiments, the virus is a wild-type virus. In some embodiments, the virus is an engineered virus. In some embodiments, the virus is an RNA virus. In some embodiments, the RNA virus is a negative-strand RNA virus. In some embodiments, the negative-strand RNA virus is a mononegavirus. In some embodiments, the mononegavirus is a rhabdovirus. In some embodiments, the rhabdovirus is a vesicular stomatitis virus (VSV). In some embodiments, the viral protein encoded by the deleted gene is VSV phosphoprotein (VSVP). In some embodiments, the first and second targets are present on the same protein. In some embodiments, the first and second targets are different epitopes of the same protein. In some embodiments, the first and second targets are present on different proteins. In some embodiments, the different proteins form a complex with each other. In some embodiments, the complex is present at a higher level in cancer cells than non-cancer cells. In some embodiments, the first and second targets are present on one or more proteins that are specifically expressed by cancer cells. In some embodiments, the first and second targets are present on one or more proteins that are expressed at a higher level in cancer cells compared to non-cancer cells. In some embodiments, the first binding domain is a phosphotyrosine binding domain. In some embodiments, the second binding domain is a phosphotyrosine binding domain. In some embodiments, the first binding domain is a phosphotyrosine binding domain and the second binding domain is a phosphotyrosine binding domain. In some embodiments, the first and second targets are in proximity to each other such that the protease cleaves the cleavable substrate when the first and second binding domains are bound to their respective targets. In some embodiments, the same protein is a receptor or a transmembrane protein. In some embodiments, the same protein is hyperactive or signals constitutively. In some embodiments, the same protein is a mutant protein. In some embodiments, the same protein is human EGF receptor 2 (HER2). In some embodiments, the same protein is epidermal growth factor receptor (EGFR). In some embodiments, the first binding domain of the first fusion protein and the second binding domain of the second fusion protein is independently selected from a Src homology 2 (SH2) domain and a Shc domain. In some embodiments, the protease is a viral protease. In some embodiments, the viral protease is a hepatitis C virus (HCV) nonstructural protein 3 (NS3) protease. In some embodiments, the membrane-targeting signal comprises a transmembrane domain. In some embodiments, the membrane-targeting signal comprises a membrane-tethering domain. In some embodiments, the membrane-tethering domain comprises a post-translational modification. In some embodiments, the post-translational modification comprises palmitoylation, myristoylation, prenylation, a glycosylphosphatidylinositol (GPI) anchor, or any combination thereof. In some embodiments, the first fusion protein, the second fusion protein, or both, comprise a protein domain that emits a detectable signal.

In an embodiment, a cell comprises a recombinant viral genome as disclosed herein. In some embodiments, the cell is a mammalian cell. In some embodiments, the ell is that of a viral packaging cell line.

In an embodiment, a virion is produced by the cell disclosed herein. In an embodiment, a virion comprises a viral genome as disclosed herein. In an embodiment a pharmaceutical composition comprises a plurality of virions.

In an embodiment, a method of treating a disease or condition in a subject in need thereof comprises administering the pharmaceutical composition, as disclosed herein. In some embodiments, the pharmaceutical composition comprises the virions in an amount effective to induce death of cells exhibiting a signal specific to the disease or disorder in the subject. In some embodiments, the pharmaceutical composition comprises the virions in an amount effective to induce death of cells exhibiting a signal that is higher than the signal in non-diseased cells. In some embodiments, the disease or condition is a cancer. In some embodiments, the disease or condition is characterized by hyperactive ErbB signaling. In some embodiments, the disease or condition characterized by hyperactive ErbB signaling is a disorder characterized by hyperactive epidermal growth factor receptor (EGFR) signaling. In some embodiments, the disease or condition characterized by hyperactive ErbB signaling is a disease or condition characterized by hyperactive human EGF receptor 2 (HER2) signaling. In some embodiments, the disease or condition characterized by hyperactive ErbB signaling is a cell proliferative disease or condition. In some embodiments, the cell proliferative disease or condition is a cancer. In some embodiments, the cancer is selected from breast cancer, ovarian cancer, and non-small cell lung cancer.

In an embodiment, a method of producing a conditionally replicating or non-replicating mononegavirus comprises infecting a first cell population with a virus expressing a DNA sequence-specific RNA polymerase and culturing a second cell population with the first cell population and/or a culture medium thereof, wherein the second cell population expresses a nucleocapsid, a phosphoprotein, and a large protein, under one or more conditions in which the second cell population produces the mononegavirus. The first cell population comprises one or more plasmids containing one or more genes encoding a nucleocapsid, a phosphoprotein, and a large protein of a mononegavirus wherein the one or more genes are linked to a DNA sequence recognized by the RNA polymerase, and a plasmid comprising a genome of the mononegavirus linked to the DNA sequence recognized by the DNA sequence-specific RNA polymerase.

In some embodiments, the DNA sequence-specific RNA polymerase is a bacteriophage T7 RNA polymerase. In some embodiments, the mononegavirus is a rhabdovirus. In some embodiments, the rhabdovirus is a VSV. In some embodiments, the first cell population comprises HEK293T cells. In some embodiments, the second cell population comprises BHK21 cells. In some embodiments, the second cell population comprises Vero cells. In some embodiments, the nucleocapsid, the phosphoprotein, or the large protein, or a combination thereof, are conditionally functional. In some embodiments, the nucleocapsid, the phosphoprotein, or the large protein, or a combination thereof, are nonfunctional.

In an embodiment, a method of producing a conditionally replicating or non-replicating virus comprises infecting a first cell population with a virus expressing a polymerase and culturing a second cell population with the first cell population and/or a culture medium thereof, wherein the second cell population expresses a nucleocapsid, a phosphoprotein, and a large protein, under one or more conditions in which the second cell population produces the virus. The first cell population comprises one or more plasmids containing one or more genes encoding a viral nucleocapsid, a viral phosphoprotein, and a viral large protein, wherein the one or more genes are linked to a DNA sequence recognized by the polymerase, and a plasmid comprising a genome of the mononegavirus linked to the DNA sequence recognized by the polymerase.

### BRIEF DESCRIPTION OF THE FIGURES

FIG. 1A recombinant adenovirus (rAd) programmed to detect oncogenic ErbB signaling and replicate only in its presence. The rAd (1) binds to the cell surface and is endocytosed (2). Bursting of the endosome allows the double-stranded DNA viral genome (3) to enter the cytosol, after which it is transported to the nucleus. Activation of a constitutive promoter by cellular proteins (4) results in transcription of mRNA (5) encoding synthetic proteins (6a and 6b) of a system for rewiring aberrant signaling to effector release (RASER). Binding of these synthetic proteins to a constitutively activated ErbB protein (7) present in cancer cells (in humans, usually EGFR and HER2), results in proteolytic release of a synthetic transcription factor cargo (8), which then activates a transcriptional cascade to produce multiple mRNAs (9) that encode proteins essential for rAd genome replication (10) and structural capsid proteins (11). New copies of the rAd genome (12) synthesized by viral and cellular replication proteins are packaged within the capsid proteins to make new rAd particles (13) which are then released when the cells die.

FIG. 2 Concept for ErbB-RASER with GAL4-VP16 transcription factor as direct output. (a) Schematic illustration of ErbB-RASER-C with GAL4-VP16 effector. GAL4-VP16 effector is fused to the C-terminus of the substrate sequence. A transmembrane domain for membrane localization is located at the very N-terminus, and the protein also contains a SH2 phosphotyrosine-binding domain N-terminal to the substrate for recruitment to active ErbB receptor. The HCV NS3 protease holoenzyme is fused to a Shc phosphotyrosine-binding domain for co-recruitment to active ErbB receptor. Released GAL4-VP16 accumulates in the presence of constitutive ErbB signaling then activates the expression of genes downstream of GAL4 UAS promoter. (b) Schematic illustration of ErbB-RASER-N with GAL4-VP16 effector. GAL4-VP16 effector is fused to the N-terminus of the substrate. A CAAX sequence for membrane localization is located at the very C-terminus, and the protein also contains a SH2 phosphotyrosine-binding domain C-terminal to the substrate for recruitment to active ErbB receptor. The HCV NS3 protease holoenzyme is fused to a Shc phosphotyrosine-binding domain for co-recruitment to active ErbB receptor. Released GAL4-VP16 accumulates in the presence of constitutive ErbB signaling then activates the expression of genes downstream of GAL4 UAS promoter.

FIG. 3 GAL4-VP16 release in an ErbB hyperactive cell line. Ad5-ErbB-RASER-N (a) and Ad5-ErbB-RASER-C (b) release GAL4-VP16 in a ErbB-dependent manner. In this case, the ErbB family member being queried is HER2. Cells were infected with lentivirus expressing ErbB-RASER-N-GAL4-VP16 and treated with or without 0.5 μM of the ErbB kinase inhibitor lapatinib. After 48 h, cells were lysed for immunoblotting against the V5 epitope tag fused to GAL4-VP16. β-Actin served as a loading control. GAL4-VP16 is released in SKBR-3 cells, a HER2-positive breast cancer cell line in a HER2 activity-dependent manner. In contrast, MCF-7 cells with normal HER2 levels did not show effector release.

FIG. 4 Schematic illustrations of the Ad5-ErbB-RASER-C (a), Ad5-ErbB-RASER-N (b), and wild-type Ad5 (c) genomes. Each rectangle represents a transcription unit. Terminator sites recognized by this complex are at the end of each transcription unit. Each arrow represents a promoter. ITR, adenovirus inverted terminal repeat CMV, CMV promoter. P2A, poliovirus ribosomal skipping element. UAS, upstream activation sites.

FIG. 5 Specificity and efficacy of Ad5-ErbB-RASER-C and Ad5-ErbB-RASER-N. Cytotoxicity of Ad5-ErbB-RASER-C (a) and Ad5-ErbB-RASER-N (b) in ErbB-hyperactive cancer cell lines (SKBR-3 and BxPC-3) or ErbB-normal cell lines (MCF-7 and Mia paCa-2). Cells were infected with Ad5-ErbB-RASER-C at the multiplicity of infection (MOI) of 10 and treated with or without 0.5 μM of the ErbB kinase inhibitor lapatinib. After 48 hours, cytotoxicity was measured by dead-cell protease activity using a luminogenic cell-impermeant peptide substrate (AAF-aminoluciferin) (CytoTox-Glo™ Assay kit, Promega). Error bars represent standard deviation, n=3 replicates.

FIG. 6 Specificity and efficacy of Ad5-ErbB-RASER-N compared to Wild-type Ad5. Cytotoxicity of Ad5-ErbB-RASER-N (a) and Wild-type Ad5 (b) in ErbB-hyperactive cancer cell lines (SKBR-3, SKOV-3 and BxPC-3) or ErbB-normal cell lines (MCF-7, MDAH2774 and Mia paCa-2). Cells were infected with Ad5-ErbB-RASER-N (a) and Wild-type Ad5 (b) at the MOI of 10 and treated with or without 0.5 μM of the ErbB kinase inhibitor lapatinib. After 6 days, viability was measured by quantifying ATP (CellTiter-Glo® 2.0 Assay kit, Promega). Error bars represent standard deviation, n=3 replicates.

FIG. 7A recombinant negative-strand RNA virus (meganegalovirus) programmed to detect oncogenic ErbB signaling and replicate only in its presence. The virus (1) binds to the cell surface and is endocytosed (2). Membrane fusion allows a viral polymerase complex (composed of nucleocapsid, phosphoprotein, and large protein) and single-stranded negative-strand viral genome (3) to enter the cytosol. The viral polymerase complex transcribes positive-strand subgenomic RNAs (4) encoding synthetic proteins (5a and 5b) of the previously described system for rewiring aberrant signaling to effector release (RASER). Binding of these synthetic proteins to a constitutively activated ErbB protein (6) present in cancer cells (in humans, usually EGFR and HER2), results in proteolytic release of multiple copies of phosphoprotein (7). At the same time, additional RNAs (8, 9) encoding nucleocapsid, large, matrix, and glycoprotein (or other viral ligand) are produced. Nucleocapsid and large (10) complex with released phosphoprotein to make copies of the complete viral genomic RNA (11), which supplies further templates for expression of all encoded proteins. Matrix and glycoprotein or other viral ligand (12) create evaginations of the membrane (13) which take up viral genomic RNAs, nucleocapsid, phosphoprotein, and large proteins and bud off as new virions.

FIG. 8 Concept for ErbB-RASER-N with measles virus phosphoprotein (MVP) or vesicular stomatitis virus phosphoprotein (VSVP) as direct output. (a) Schematic description of ErbB-RASER-N with MVP as effector. MVP is fused to the N-terminus of the substrate, with a C-terminal CAAX sequence for membrane localization. VP is released in the presence of constitutive ErbB signaling then participates in genome replication and viral gene expression. (b) Schematic description of ErbB-RASER-N with VSVP as effector. VSVP is fused to the N-terminus of the substrate, with a C-terminal CAAX sequence for membrane localization. VSVP is released in the presence of constitutive ErbB signaling then participates in genome replication and viral gene expression.

FIG. 9 MVP or VSVP release by RASER systems in ErbB hyperactive cells. (a) ErbB-RASER-N releases MVP in a ErbB activity-dependent manner. Cells were transfected with plasmids expressing ErbB-RASER-N-MVP and treated with or without 50 nM of the ErbB kinase inhibitor lapatinib. After 24 h, cells were lysed for immunoblotting against the V5 epitope tag fused to MVP. MVP was released in LN299 cells overexpressing EGFRvlll in the absence of lapatinib, but release was inhibited by lapatinib. (b) ErbB-RASER-N releases VSVP in a ErbB activity-dependent manner. Cells were infected with lentivirus expressing ErbB-RASER-N-VSVP and treated with or without 500 nM of the ErbB kinase inhibitor lapatinib. After 48 h, cells were lysed for immunoblotting against the V5 epitope tag fused to VSVP. β-Actin served as a loading control. Release of VSVP is observed in SKBR-3 cells, a HER2-positive breast cancer cell line in an ErbB-dependent manner. In contrast, MCF-7 cells with normal ErbB did not show VSVP release.

FIG. 10 Schematic of (a) MV-ErbB-RASER-N and MV-GFP and (b) VSV-ErbB-RASER-N and VSV-YFP genomes. For MV-ErbB-RASER-N (a): Each rectangle represents a transcription unit. Triangles denote transcription start sites recognized by the RNA-dependent polymerase complex of large, phosphoprotein, and nucleocapsid. Terminator sites recognized by this complex are at the end of each transcription unit. GFP, green fluorescent protein. N, nucleocapsid. M, matrix. F, fusion. H, hemagglutinin. L, large. MVP, MV phosphoprotein. A transcriptional unit expressing unfused MVP no longer exists, rendering virus replication conditional on MVP release from ErbB-RASER-N-MVP. Other arrangements of the coding sequences are possible, as is the addition of other coding sequences. Note the coding sequence for the fluorescent protein can be removed. For VSV-ErbB-RASER-N (b): Each rectangle represents a transcription unit. Triangles denote transcription start sites recognized by the RNA-dependent polymerase complex of large, phosphoprotein, and nucleocapsid. Terminator sites recognized by this complex are at the end of each transcription unit. YFP, yellow fluorescent protein. N, nucleocapsid. M, matrix. G/Ligand, glycoprotein or other viral ligand. L, large. VSVP, VSV phosphoprotein. P2A, poliovirus ribosomal skipping element. A transcriptional unit expressing unfused MVP no longer exists, rendering virus replication conditional on MVP release from ErbB-RASER-N-MVP. Other arrangements of the coding sequences are possible, as is the addition of other coding sequences. Note the coding sequence for the fluorescent protein can be removed.

FIG. 11 Unsuccessful rescue (or recovery, i.e., packaging) of MV-ErbB-RASER-N according to a prior art method, and successful rescue of VSV-ErbB-RASER-N by a method according to embodiments of the present disclosure. (a) A published and widely used MV rescue procedure (Parks et al. 2000 J Virol 73:3560) was unsuccessful in rescuing MV-ErbB-RASER-N virus in 3 laboratories, but was successful in rescuing MV-GFP, as published previously (Chung et al. 2015, Nat Chem Biol 11:713) (b) A VSV rescue procedure according to embodiments of the present disclosure was successful in rescuing VSV-ErbB-RASER-N with similar virus yield as VSV-YFP. One previously described procedure (Takahasi and Yokobayashi, 2019 ACS Synth Biol 8:1976) transfected HEK293 cells with plasmids expressing T7 polymerase and plasmids expressing a modified VSV genome and the N, P, and L proteins from T7 promoters for initial virus rescue, then transferred supernatant to BHK21 cells for further amplification of any rescued viral particles. Another previously described procedure (Ammayappan et al, 2013 J Virol 87:3217) infected BHK21 cells with vaccinia virus expressing the T7 polymerase and transfected into the same cells plasmids expressing a modified VSV genome and the N, P, and L proteins from T7 promoters. We instead infected HEK293T cells with vaccinia virus expressing the T7 polymerase and transfected plasmids expressing the modified VSV genome and the N, P, and L proteins from T7 promoters, for the initial rescue. For viral amplification, we created BHK21 cells stably expressing the N, P, and L proteins so that VSV-ErbB-RASER-N viruses entering these cells do not need to produce their own N or L, or release P from the RASER substrate protein, to initiate the early steps of the virus life cycle. We overlaid these cells on to the HEK293T cells 2 days after vaccinia infection and plasmid transfection as described above. This method was successful in rescuing (or recovering) the VSV-ErbB-RASER-N virus to similarly high titers as VSV-GFP, which replicates without growth restriction.

FIG. 12 Specificity and efficacy of VSV-ErbB-RASER-N. Cytotoxicity of VSV-ErbB-RASER-N in ErbB-hyperactive cancer cell lines (SKBR-3 and BxPC-3) or ErbB-normal cell lines (MCF-7 and Mia paCa-2). Cells were infected with Ad5-ErbB-RASER-C at the MOI of 1 and treated with or without 0.5 μM of the ErbB kinase inhibitor lapatinib. After 48 hours, cytotoxicity was measured by dead-cell protease activity using a luminogenic cell-impermeant peptide substrate (AAF-aminoluciferin) (CytoTox-Glo™ Assay kit, Promega). Error bars represent standard deviation, n=3 replicates.

FIG. 13 Specificity and efficacy of VSV-ErbB-RASER-N compared to VSV-YFP. Cytotoxicity of VSV-ErbB-RASER-N (a) and Wild-type VSV (b) in ErbB-hyperactive cancer cell lines (SKBR-3, SKOV-3 and BxPC-3) or ErbB-normal cell lines (MCF-7, MDAH2774 and Mia paCa-2). Cells were infected with VSV-ErbB-RASER-N (a) and Wild-type VSV (b) at the MOI of 1 and treated with or without 0.5 μM of the ErbB kinase inhibitor lapatinib. After 4 days, viability was measured by quantifying ATP (CellTiter-Glo® 2.0 Assay kit, Promega). Error bars represent standard deviation, n=3 replicates.

FIG. 14 Fluorescence microscopy of VSV-ErbB-RASER-N replication in ErbB-hyperactive cancer cell lines (SKBR-3) or ErbB-normal cell lines (MCF-7). Cells were infected with VSV-ErbB-RASER-N at the MOI of 1 and treated with or without 0.5 μM of the ErbB kinase inhibitor lapatinib. Images were taken 48 hours infection. Scale bars, 400μm.

### DETAILED DESCRIPTION

Before the recombinant viral genomes, compositions and methods of the present disclosure are described in greater detail, it is to be understood that the recombinant viral genomes, compositions and methods are not limited to particular embodiments described, as such may, of course, vary. It is also to be understood that the terminology used herein is for the purpose of describing particular embodiments only, and is not intended to be limiting, since the scope of the recombinant viral genomes, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the recombinant viral genomes, compositions and methods. The upper and lower limits of these smaller ranges may independently be included in the smaller ranges and are also encompassed within the recombinant viral genomes, compositions and methods, subject to any specifically excluded limit in the stated range. Where the stated range includes one or both of the limits, ranges excluding either or both of those included limits are also included in the recombinant viral genomes, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the recombinant viral genomes, compositions and methods belong. Although any recombinant viral genomes, compositions and methods similar or equivalent to those described herein can also be used in the practice or testing of the recombinant viral genomes, compositions and methods, representative illustrative recombinant viral genomes, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present recombinant viral genomes, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the recombinant viral genomes, compositions and methods, which are, for clarity, described in the context of separate embodiments, may also be provided in combination in a single embodiment. Conversely, various features of the recombinant viral genomes, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present recombinant viral genomes, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Recombinant Viral Genomes

The present disclosure provides recombinant viral genomes. As summarized above, the viral genomes find use in a variety of contexts including for the production of viruses effective in inducing death of cells exhibiting a signal specific to a disease or disorder in a subject, where the viral life cycle is dependent upon the signal specific to the disease or disorder. According to some embodiments, the viral genomes comprise one or more transcription units comprising a sequence encoding a first fusion protein comprising a protease and a first binding domain that binds to a first target, and a sequence encoding a second fusion protein comprising a membrane-targeting signal, a second binding domain that binds to a second target, a cleavable substrate for the protease, and either a transcriptional effector protein or viral protein encoded by a viral gene deleted in the viral genome. Details regarding the recombinant viral genomes of the present disclosure will now be described.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide.

A first aspect of the present disclosure includes recombinant viral genomes comprising a first transcription unit and a second transcription unit. The first transcription unit comprises a sequence encoding a first fusion protein comprising a protease and a first binding domain that binds to a first target. The first transcription unit further comprises a sequence encoding a second fusion protein comprising, in N-terminal to C-terminal order or in C-terminal to N-terminal order, a membrane-targeting signal, a second binding domain that binds to a second target, a cleavable substrate for the protease, and a transcriptional effector protein. Such recombinant viral genomes further comprise a second transcription unit comprising a sequence encoding one or more viral replication proteins, wherein the second transcription unit is operably linked to a promoter activatable by the transcriptional effector protein. In certain embodiments, the recombinant viral genome comprises a modified genome of a virus.

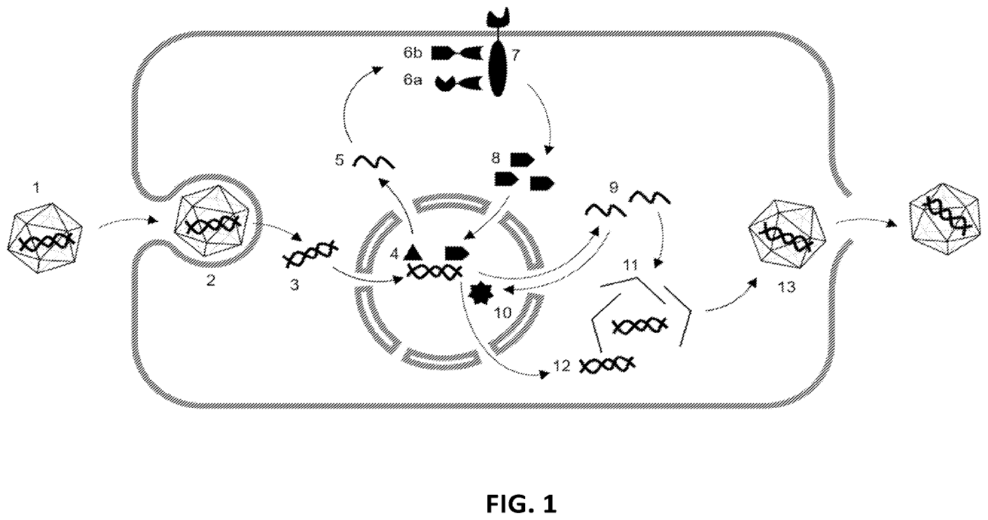

The above first aspects are applicable to a variety of types of viruses, non-limiting examples of which include oncolytic viruses, adenoviruses, etc. In general, these aspects are applicable to viruses (e.g., double-stranded DNA viruses, such as adenoviruses) having a life cycle involving entry of the viral genome into the nucleus followed by transcription of genes within the viral genome by host RNA polymerases. For example, in certain embodiments, a recombinant viral genome of the present disclosure is a modified genome of a virus having a life cycle that begins with its DNA genome entering the cell nucleus, where a set of early regulatory genes are transcribed into messenger RNAs by host RNA polymerases. The regulatory proteins produced then induce later expression of genes encoding DNA polymerase and structural proteins. The virus DNA is then replicated and packaged into progeny virions inside the cytoplasm, which are then released as cells die, primarily through necrosis. A non-limiting example of a virus having such a life cycle and a recombinant viral genome according to embodiments of the present disclosure is schematically illustrated in FIG. 1. As shown: the virus (1) binds to the cell surface and is endocytosed (2); bursting of the endosome allows the double-stranded DNA viral genome (3) to enter the cytosol, after which it is transported to the nucleus; activation of a constitutive promoter by cellular proteins (4) results in transcription of mRNA (5) encoding the first and second fusion proteins (6a and 6b); binding of the fusion proteins to their respective targets on protein (7) (responsible at least in part for a signal specific to a disease or disorder) results in proteolytic release of transcriptional effector protein (8), which then activates a transcriptional cascade to produce multiple mRNAs (9) that encode proteins essential for viral genome replication (10) and structural capsid proteins (11). New copies of the recombinant genome (12) synthesized by viral and cellular replication proteins are packaged within the capsid proteins to make new virus particles (virions) (13) which are then released when the cells die.

In certain embodiments of the first aspects, the promoter is not activatable by the transcriptional effector protein until the transcriptional effector protein is cleaved from the second fusion protein. According to some embodiments of the first aspects, the promoter is activatable to a lesser extent by the second fusion protein compared to the transcriptional effector protein when cleaved from the second fusion protein. In certain embodiments of the first aspects, the transcriptional effector protein comprises a transcription factor.

According to some embodiments of the first aspects, the one or more viral replication proteins encoded by the second transcription unit is required for replication of the virus in cells or efficient replication of the virus in cells. In certain embodiments of the first aspects, replication of the virus in cells is dependent on a signal specific to cancer cells or a signal in cancer cells that is higher than the signal in non-cancer cells. According to some embodiments of the first aspects, replication of the virus in cells is not dependent on the presence of an endogenous transcription factor. In certain embodiments, the endogenous transcription factor is an endogenous cancer-selective transcription factor.

In certain embodiments of the first aspects, the recombinant viral genome further comprises a third transcription unit encoding an immunostimulatory molecule. A non-limiting example of an immunostimulatory molecule that find use in the context of the present disclosure is granulocyte/macrophage colony stimulating factor (GM-CSF).

According to some embodiments of the first aspects, the recombinant viral genome comprises a modified genome of a virus, where the virus is a double-stranded DNA virus. A non-limiting example of such a virus is an adenovirus. When the recombinant viral genome comprises a modified genome of an adenovirus, in certain embodiments, the one or more viral replication proteins comprise one or more E1 gene products.

In certain embodiments of the first aspects, the transcriptional effector protein comprises a GAL4-VP16 transcription factor. When the transcriptional effector protein comprises a GAL4-VP16 transcription factor, according to some embodiments, the promoter activatable by the transcriptional effector protein is a GAL4 UAS promoter.

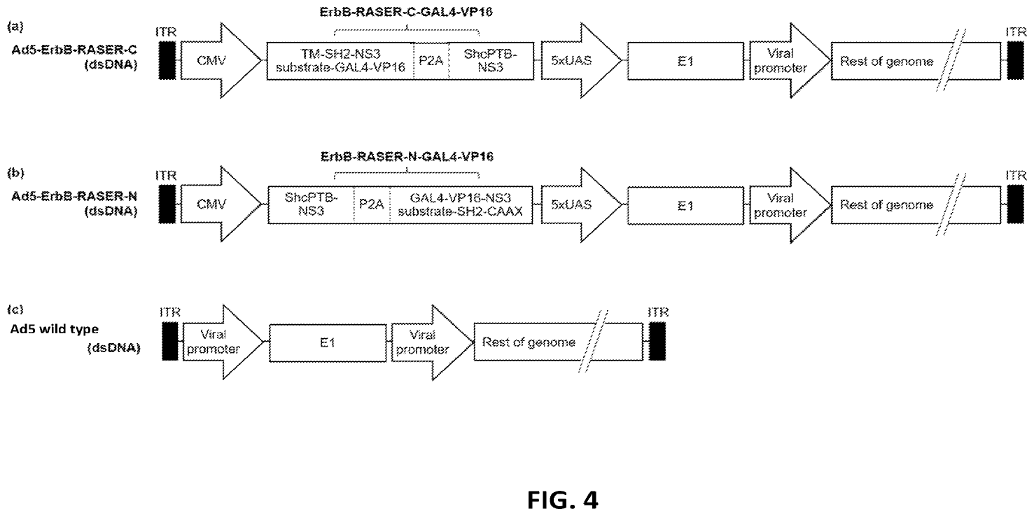

Non-limiting examples of recombinant viral genomes according to embodiments of the first aspects are schematically illustrated in FIG. 4. Shown are Ad5-ErbB-RASER-C (a), Ad5-ErbB-RASER-N (b), and wild-type Ad5 (c) genomes. Each rectangle represents a transcription unit. Terminator sites recognized by this complex are at the end of each transcription unit. Each arrow represents a promoter. ITR, adenovirus inverted terminal repeat CMV, CMV promoter. P2A, poliovirus ribosomal skipping element. UAS, upstream activation sites. Nucleotide sequences from the CMV promoter through the 5xUAS sequence for the Ad5-ErbB-RASER-C and Ad5-ErbB-RASER-N constructs, as well as the amino acid sequences of the first and second fusion proteins encoded by these constructs, are provided in Table 1 below.

TABLE 1
Ad5-ErbB-RASER-C and Ad5-ErbB-RASER-N Nucleotide and Amino Acid
Sequences

| | |
|---|---|
| Ad5-ErbB-RASER-C | actgtaatagtaatcaattacggggtcattagttcatagcccatatatggagttccgcgttacata |
| (SEQ ID NO: 1) | acttacggtaaatggcccgcctggctgaccgcccaacgacccccgcccattgacgtcaata |
| CMV promoter::TM-SH2- | atgacgtatgttcccatagtaacgccaatagggactttccattgacgtcaatgggtggagtattt |
| NS3-substrate-GAL4- | acggtaaactgcccacttggcagtacatcaagtgtatcatatgccaagtacgccccctattga |
| VP16-P2A-ShcPTB- | cgtcaatgacggtaaatggcccgcctggcattatgcccagtacatgaccttatgggactttcct |
| NS3::polyadenylation | acttggcagtacatctacgtattagtcatcgctattaccatggtgatgcggttttggcagtacatc |
| signal::5xUAS promoter | aatgggcgtggatagcggtttgactcacggggatttccaagtctccaccccattgacgtcaat |
| (replaces nucleotides | gggagtttgttttggcaccaaaatcaacgggactttccaaaatgtcgtaacaactccgccccat |
| 342-541 of wild-type Ad5 | tgacgcaaatgggcggtaggcgtgtacggtgggaggtctatataagcagagctggtttagtg |
| (Genbank accession # | aaccgtcagatccgctagagatctggtaccgtcgaccaccatgcacctgagaatccacgcg |
| AC_000008) to create | agacggagccctcctcgccggccggcctggacgcttgggatctggttcctgttctggggatgt |
| Ad5-ErbB-RASER-C. And | atcgtcagctctgtatggagtcaattgtcttctaatgtagcttcctcctcctccacctcttcctcgc |
| Deletion of Nucleotide | cggggtctcactacccatacgatgtgccagattacgccaaccccacggagccgggaatcag |
| 28137-30818 from wild- | acgggttccgggggcctcagaggtgatccgggagtcgagcagcacaacagggatggtcgt |
| type Ad5 (Genbank | cggcattgtggctgctgccgccctctgcatcttgatcctcctgtacgccatgtacaagtacagg |
| accession # AC_000008)) | aacagggacgagggctcgggctcgacctcgggctcgggcagcggtggctctggaggttct |
| | ggtggcagctctggaggtatgaatggagctataggaggtgaccttttgctcaattttcctgacat |
| | gtcggtcctagagcgccaaagggctcacctcaagtacctcaatcccacctttgattctcctctc |
| | gccggcttctttgccgattcttcaatgattaccggcggcgagatggacagctatctttcgactgc |
| | cggtttgaatcttccgatgatgtacggtgagacgacggtggaaggtgattcaagactctcaatt |
| | tcgccggaaacgacgcttgggactggaaatttcaaggcagcgaagtttgatacagagacta |
| | aggattgtaatgaggcggcgaagaagatgacgatgaacagagatgacctagtagaagaa |
| | ggagaagaagagaagtcgaaaataacagagcaaaacaatgggagcacaaaaagcatc |
| | aagaagatgaaacacaaagccaagaaagaagagaacaatttctctaatgattcatctaaa |
| | gtgacgaaggaattggagaaaacggattatattcatagcggtagtggggcggcagtgggg |
| | gctctggtgggtcaggtggcagtggggccctcctcaggacctgtctgttcatctctggtacgc |
| | aggccccatggagcgggcaggggcagagagcatcctggccaaccgctcggacgggactt |
| | tcttggtgcggcagagggtgaaggatgcagcagaatttgccatcagcattaaatataacgtc |
| | gaggtcaagcacattaaaatcatgacagcagaaggactgtaccggatcacagagaaaaa |
| | ggctttccgggggcttacggagctggtggagttttaccagcagaactctctaaaggattgcttc |
| | aagtctctggacaccaccttgcagttccccttcaaggagcctgaaaagagaggatctagag |
| | ggggatctggtggatcagggggtagcgggggttctggcggaccggcaggcagtagcgga |
| | agcagtattatacctgacagggaggttctctaccaggagttcgatgagatggaagagtgctct |
| | cagcacctccccggcgcgcctgctagctctatcgaacaagcatgcgatatttgccgacttaaa |
| | aagctcaagtgctccaaagaaaaaccgaagtgcgccaagtgtctgaagaacaactggga |
| | gtgtcgctactctcccaaaaccaaaaggtctccgctgactagggcacatctgacagaagtgg |
| | aatcaaggctagaaagactggaacagctatttctactgatttttcctcgagaagaccttgacat |
| | gattttgaaaatggattctttacaggatataaaagcattgttaacaggattatttgtacaagataa |
| | tgtgaataaagatgccgtcacagatagattggcttcagtggagactgatatgcctctaacattg |
| | agacagcatagaataagtgcgacatcatcatcggaagagagtagtaacaaaggtcaaag |
| | acagttgactgtatcgatgggccctaaaaagaagcgtaaagtcgcccccccgaccgatgtc |
| | agcctgggggacgagctccacttagacggcgaggacgtggcgatggcgcatgccgacgc |
| | gctagacgatttcgatctggacatgttgggggacggggattccccgggtccgggatttacccc |
| | ccacgactccgccccctacggcgctctggatatggccgacttcgagtttgagcagatgtttacc |
| | gatgcccttggaattgacgagtacggtgggattaacggcagtggagagggcagaggaagt |
| | ctgctaacatgcggtgacgtcgaggagaatcctggcccaatgggcaagccactgcatccca |
| | acgacaaagtcatgggacccggggtttcctacttggttcggtacatgggttgtgtggaggtcct |
| | ccagtcaatgcgtgccctggacttcaacacccggactcaggtcaccagggaggccatcagt |
| | ctggtgtgtgaggctgtgccgggtgctaagggggcgacaaggaggagaaagccctgtagc |
| | cgcccgctcagctctatcctggggaggagtaacctgaaatttgctggaatgccaatcactctc |
| | accgtctccaccagcagcctcaacctcatggccgcagactgcaaacagatcatcgccaac |
| | caccacatgcaatctatctcatttgcatccggcatgttagctccctatatcccagagtatgtcgcc |
| | tatgttgccaaagaccctgtgaatcagagagcctgccacattctggagtgtcccgaagggctt |
| | gcccaggatgtcatcagcaccattggccaggccttcgagttgcgcttcaaacaatacctcag |
| | ggacatcgagcaggtgccccagcagcccacccttaagatggattacaaggatgacgacga |
| | taagggctcttccgggacaggctccggatccggcactagtgcgcccatcacggcgtacgcc |
| | cagcagacgagaggcctcctagggtgtataatcaccagcctgactggccgggacaaaaac |
| | caagtggagggtgaggtccagatcgtgtcaactgctacccaaaccttcctggcaacgtgcat |
| | caatggggtatgctggactgtctaccacggggccggaacgaggaccatcgcatcacccaa |
| | gggtcctgtcatccagatgtataccaatgtggaccaagaccttgtgggctggcccgctcctca |
| | aggttcccgctcattgacaccctgtacctgcggctcctcggacctttacctggtcacgaggcac |
| | gccgatgtcattcccgtgcgccggcgaggtgatagcaggggtagcctgctttcgccccggcc |
| | catttcctacttgaaaggctcctcggggggtccgctgttgtgccccgcgggacacgccgtggg |
| | cctattcagggccgcggtgtgcacccgtggagtggctaaagcggtggactttatccctgtgga |
| | gaacctagagacaaccatgagatccccggtgttctcggacaattcctccccaccagcggtgt |
| | gagggccgctcgagcctaagcttctagataagatatccgatccaccggatctagataactga |
| | tcataatcagccataccacatttgtagaggttttacttgctttaaaaaacctcccacacctccccc |
| | tgaacctgaaacataaaatgaatgcaattgttgttgttaacttgtttattgcagcttataatggtta |
| | caaataaagcaatagcatcacaaatttcacaaataaagcatttttttcactgcattctagttgtggt |
| | ttgtccaaactcatcaatgtatcttacctgcaggtcggagtactgtcctccgagcggagtactgt |
| | cctccgagcggagtactgtcctccgagcggagtactgtcctccgagcggagtactgtcctccg |
| | agcggagactctagcgagcgcgtactgccgccaccatgg |
| Ad5-ErbB-RASER-C | MHLRIHARRSPPRRPAWTLGIWFLFWGCIVSSVWSQLSSNVASS |
| (SEQ ID NO: 2) | SSTSSSPGSHYPYDVPDYANPTEPGIRRVPGASEVIRESSSTTGM |
| TM-SH2-NS3-substrate- | VVGIVAAAALCILILLYAMYKYRNRDEGSGSTSGSGSGGSGGSGG |
| GAL4-VP16-P2A | SSGGMNGAIGGDLLLNFPDMSVLERQRAHLKYLNPTFDSPLAGFF |
| | ADSSMITGGEMDSYLSTAGLNLPMMYGETTVEGDSRLSISPETTL |
| | GTGNFKAAKFDTETKDCNEAAKKMTMNRDDLVEEGEEEKSKITE |
| | QNNGSTKSIKKMKHKAKKEENNFSNDSSKVTKELEKTDYIHSGSG |
| | GGSGGSGGSGGSGGPPQDLSVHLWYAGPMERAGAESILANRSD |
| | GTFLVRQRVKDAAEFAISIKYNVEVKHIKIMTAEGLYRITEKKAFRG |
| | LTELVEFYQQNSLKDCFKSLDTTLQFPFKEPEKRGSRGGSGGSG |
| | GSGGSGGPAGSSGSSIIPDREVLYQEFDEMEECSQHLPGAPASSI |
| | EQACDICRLKKLKCSKEKPKCAKCLKNNWECRYSPKTKRSPLTRA |
| | HLTEVESRLERLEQLFLLIFPREDLDMILKMDSLQDIKALLTGLFVQ |
| | DNVNKDAVTDRLASVETDMPLTLRQHRISATSSSEESSNKGQRQL |
| | TVSMGPKKKRKVAPPTDVSLGDELHLDGEDVAMAHADALDDFDL |
| | DMLGDGDSPGPGFTPHDSAPYGALDMADFEFEQMFTDALGIDEY |
| | GGIN |
| Ad5-ErbB-RASER-C | MGKPLHPNDKVMGPGVSYLVRYMGCVEVLQSMRALDFNTRTQV |
| (SEQ ID NO: 3) | TREAISLVCEAVPGAKGATRRRKPCSRPLSSILGRSNLKFAGMPIT |
| ShcPTB-NS3 | LTVSTSSLNLMAADCKQIIANHHMQSISFASGMLAPYIPEYVAYVA |
| | KDPVNQRACHILECPEGLAQDVISTIGQAFELRFKQYLRDIEQVPQ |
| | QPTLKMDYKDDDDKGSSGTGSGSGTSAPITAYAQQTRGLLGCIIT |
| | SLTGRDKNQVEGEVQIVSTATQTFLATCINGVCWTVYHGAGTRTI |
| | ASPKGPVIQMYTNVDQDLVGWPAPQGSRSLTPCTCGSSDLYLVT |
| | RHADVIPVRRRGDSRGSLLSPRPISYLKGSSGGPLLCPAGHAVGL |
| | FRAAVCTRGVAKAVDFIPVENLETTMRSPVFSDNSSPPAV |
| Ad5-ErbB-RASER-N | actgtaatagtaatcaattacggggtcattagttcatagcccatatatggagttccgcgttacata |
| (SEQ ID NO: 4) | acttacggtaaatggcccgcctggctgaccgcccaacgacccccgcccattgacgtcaata |
| CMV promoter::ShcPTB- | atgacgtatgttcccatagtaacgccaatagggactttccattgacgtcaatgggtggagtattt |
| NS3-P2A-GAL4-VP16- | acggtaaactgcccacttggcagtacatcaagtgtatcatatgccaagtacgccccctattga |
| NS3-substrate SH2- | cgtcaatgacggtaaatggcccgcctggcattatgcccagtacatgaccttatgggactttcct |
| CAAX::polyadenylation | acttggcagtacatctacgtattagtcatcgctattaccatggtgatgcggttttggcagtacatc |
| signal::5xUAS promoter | aatgggcgtggatagcggtttgactcacggggatttccaagtctccaccccattgacgtcaat |
| (replaces nucleotides | gggagtttgttttggcaccaaaatcaacgggactttccaaaatgtcgtaacaactccgccccat |
| 342-541 of wild-type Ad5 | tgacgcaaatgggcggtaggcgtgtacggtgggaggtctatataagcagagctggtttagtg |
| (Genbank accession # | aaccgtcagatccgctagagatctggtaccgtcgacgccaccatgggcaagccactgcatc |
| AC_000008) to create | ccaacgacaaagtcatgggacccggggtttcctacttggttcggtacatgggttgtgtggaggt |
| Ad5-ErbB-RASER-N. And | cctccagtcaatgcgtgccctggacttcaacacccggactcaggtcaccagggaggccatc |
| Deletion of Nucleotide | agtctggtgtgtgaggctgtgccgggtgctaagggggcgacaaggaggagaaagccctgt |
| 28137-30818 from wild- | agccgcccgctcagctctatcctggggaggagtaacctgaaatttgctggaatgccaatcact |
| type Ad5 (Genbank | ctcaccgtctccaccagcagcctcaacctcatggccgcagactgcaaacagatcatcgcca |
| accession # AC_000008)) | accaccacatgcaatctatctcatttgcatccggcatgttagctccctatatcccagagtatgtc |
| | gcctatgttgccaaagaccctgtgaatcagagagcctgccacattctggagtgtcccgaagg |
| | gcttgcccaggatgtcatcagcaccattggccaggccttcgagttgcgcttcaaacaatacct |
| | cagggacatcgagcaggtgccccagcagcccacccttaagatggattacaaggatgacga |
| | cgataagggctcttccgggacaggctccggatccggcactagtgcgcccatcacggcgtac |
| | gcccagcagacgagaggcctcctagggtgtataatcaccagcctgactggccgggacaaa |
| | aaccaagtggagggtgaggtccagatcgtgtcaactgctacccaaaccttcctggcaacgtg |
| | catcaatggggtatgctggactgtctaccacggggccggaacgaggaccatcgcatcaccc |
| | aagggtcctgtcatccagatgtataccaatgtggaccaagaccttgtgggctggcccgctcct |
| | caaggttcccgctcattgacaccctgtacctgcggctcctcggacctttacctggtcacgaggc |
| | acgccgatgtcattcccgtgcgccggcgaggtgatagcaggggtagcctgctttcgccccgg |
| | cccatttcctacttgaaaggctcctcggggggtccgctgttgtgccccgcgggacacgccgtg |
| | ggcctattcagggccgcggtgtgcacccgtggagtggctaaagcggtggactttatccctgtg |
| | gagaacctagagacaaccatgagatccccggtgttctcggacaattcctccccaccagcgg |
| | tgggctctggagccaccaacttcagcctgctgaagcaggcaggcgacgtggaagagaacc |
| | ctggccctgctagctctatcgaacaagcatgcgatatttgccgacttaaaaagctcaagtgctc |
| | caaagaaaaaccgaagtgcgccaagtgtctgaagaacaactgggagtgtcgctactctcc |
| | caaaaccaaaaggtctccgctgactagggcacatctgacagaagtggaatcaaggctaga |
| | aagactggaacagctatttctactgatttttcctcgagaagaccttgacatgattttgaaaatgg |
| | attctttacaggatataaaagcattgttaacaggattatttgtacaagataatgtgaataaagat |
| | gccgtcacagatagattggcttcagtggagactgatatgcctctaacattgagacagcataga |
| | ataagtgcgacatcatcatcggaagagagtagtaacaaaggtcaaagacagttgactgtat |
| | cgatgggccctaaaaagaagcgtaaagtcgcccccccgaccgatgtcagcctgggggac |
| | gagctccacttagacggcgaggacgtggcgatggcgcatgccgacgcgctagacgatttcg |
| | atctggacatgttgggggacggggattccccgggtccgggatttaccccccacgactccgcc |
| | ccctacggcgctctggatatggccgacttcgagtttgagcagatgtttaccgatgcccttggaat |
| | tgacgagtacggtgggcttaagggatccggtaaaccaatccccaatcctctgctgggtcttga |
| | tagcaccgatgagatggaagagtgctctcagcacctccccggcggttcgggaggttctggtg |
| | ggtcaggtggcagtggcggccctcctcaggacctgtctgttcatctctggtacgcaggcccca |
| | tggagcgggcaggggcagagagcatcctggccaaccgctcggacgggactttcttggtgc |
| | ggcagagggtgaaggatgcagcagaatttgccatcagcattaaatataacgtcgaggtcaa |
| | gcacattaaaatcatgacagcagaaggactgtaccggatcacagagaaaaaggctttccg |
| | ggggcttacggagctggtggagttttaccagcagaactctctaaaggattgcttcaagtctctg |
| | gacaccaccttgcagttccccttcaaggagcctgaaaagagaggatctagagggggatctg |
| | gtggatcagggggtagcgggggttctggcggacaattcaagatgagcaaggacggcaag |
| | aagaaaaagaagaagtccaagactaagtgcgtgatcatgtgagggccgctcgagcctaa |
| | gcttctagataagatatccgatccaccggatctagataactgatcataatcagccataccacat |
| | ttgtagaggttttacttgctttaaaaaacctcccacacctccccctgaacctgaaacataaaatg |
| | aatgcaattgttgttgttaacttgtttattgcagcttataatggttacaaataaagcaatagcatca |
| | caaatttcacaaataaagcatttttttcactgcattctagttgtggtttgtccaaactcatcaatgt |
| | atcttacctgcaggtcggagtactgtcctccgagcggagtactgtcctccgagcggagtactgtc |
| | ctccgagcggagtactgtcctccgagcggagtactgtcctccgagcggagactctagcgag |
| | cgcgtactgccgccaccatgg |
| Ad5-ErbB-RASER-N | ASSIEQACDICRLKKLKCSKEKPKCAKCLKNNWECRYSPKTKRSP |
| (SEQ ID NO: 5) | LTRAHLTEVESRLERLEQLFLLIFPREDLDMILKMDSLQDIKALLTG |
| GAL4-VP16-NS3- | LFVQDNVNKDAVTDRLASVETDMPLTLRQHRISATSSSEESSNKG |
| substrate SH2-CAAX | QRQLTVSMGPKKKRKVAPPTDVSLGDELHLDGEDVAMAHADALD |
| | DFDLDMLGDGDSPGPGFTPHDSAPYGALDMADFEFEQMFTDAL |
| | GIDEYGGLKGSGKPIPNPLLGLDSTDEMEECSQHLPGGSGGSGG |
| | SGGSGGPPQDLSVHLWYAGPMERAGAESILANRSDGTFLVRQR |
| | VKDAAEFAISIKYNVEVKHIKIMTAEGLYRITEKKAFRGLTELVEFYQ |
| | QNSLKDCFKSLDTTLQFPFKEPEKRGSRGGSGGSGGSGGSGGQ |
| | FKMSKDGKKKKKKSKTKCVIM |
| Ad5-ErbB-RASER-N | MGKPLHPNDKVMGPGVSYLVRYMGCVEVLQSMRALDFNTRTQV |
| (SEQ ID NO: 3) | TREAISLVCEAVPGAKGATRRRKPCSRPLSSILGRSNLKFAGMPIT |
| ShcPTB-NS3 | LTVSTSSLNLMAADCKQIIANHHMQSISFASGMLAPYIPEYVAYVA |
| | KDPVNQRACHILECPEGLAQDVISTIGQAFELRFKQYLRDIEQVPQ |
| | QPTLKMDYKDDDDKGSSGTGSGSGTSAPITAYAQQTRGLLGCIIT |
| | SLTGRDKNQVEGEVQIVSTATQTFLATCINGVCWTVYHGAGTRTI |
| | ASPKGPVIQMYTNVDQDLVGWPAPQGSRSLTPCTCGSSDLYLVT |
| | RHADVIPVRRRGDSRGSLLSPRPISYLKGSSGGPLLCPAGHAVGL |
| | FRAAVCTRGVAKAVDFIPVENLETTMRSPVFSDNSSPPAV |

A second aspect of the present disclosure includes recombinant viral genomes comprising a deletion of a viral gene, and one or more transcription units. The one or more transcription units comprise a sequence encoding a first fusion protein comprising a protease and a first binding domain that binds to a first target. The one or more transcription units further comprise a sequence encoding a second fusion protein comprising, in N-terminal to C-terminal order or in C-terminal to N-terminal order, a membrane-targeting signal, a second binding domain that binds to a second target, a cleavable substrate for the protease, and the viral protein encoded by the deleted gene. In certain embodiments, the recombinant viral genome comprises a modified genome of a virus. The recombinant viral genomes according to the second aspects are applicable to a variety of types of viruses, non-limiting examples of which include RNA viruses, including but not limited to negative-strand RNA viruses, e.g., vesicular stomatitis virus (VSV) or the like.

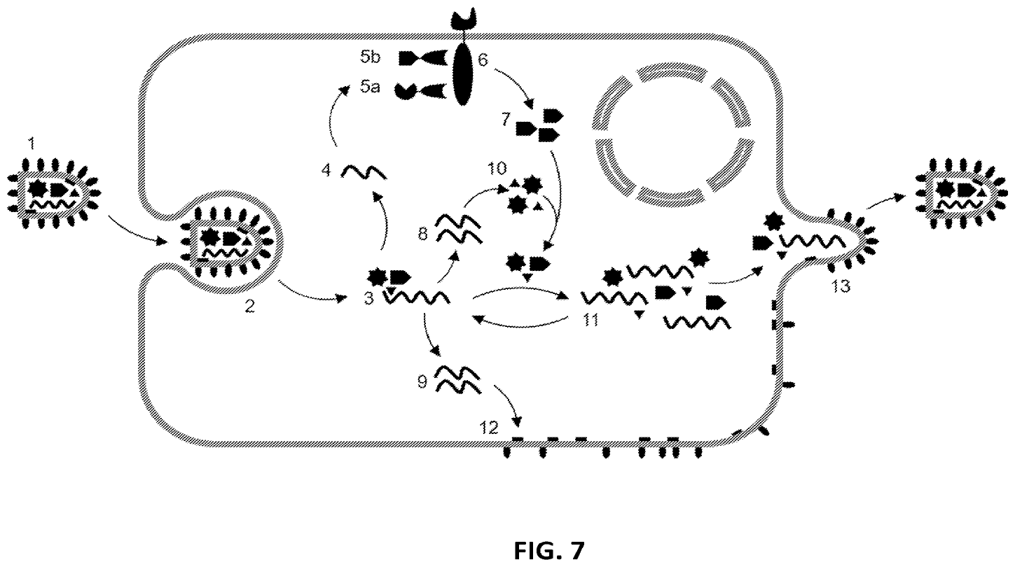

In certain embodiments, a recombinant viral genome according to the second aspects comprise a modified viral genome of a virus having a life cycle involving entry into the cytoplasm of a negative-strand RNA genome and a small number of replicase complexes, and transcription of messenger RNAs from the viral genes by the replicase (e.g., the five viral genes of VSFV). The resulting newly synthesized replicase complexes and structural proteins then allow formation of a full-length positive RNA genome strand, followed by amplification of full-length progeny negative genomes, recruitment of genomes to the membrane, and budding of new virions from the membrane, resulting in cell death primarily through necrosis. A non-limiting example of a virus having such a life cycle and a recombinant viral genome according to embodiments of the present disclosure is schematically illustrated in FIG. 7. This example depicts a recombinant negative-strand RNA virus (meganegalovirus) engineered to replicate only in the presence of a signal specific to a disease or disorder. In particular, in this example, the virus (1) binds to the cell surface and is endocytosed (2). Membrane fusion allows a viral polymerase complex (composed of nucleocapsid, phosphoprotein, and large protein) and single-stranded negative-strand viral genome (3) to enter the cytosol. The viral polymerase complex transcribes positive-strand subgenomic RNAs (4) encoding the first and second fusion proteins (5a and 5b). Binding of the fusion proteins to their respective targets on protein (6) (responsible at least in part for a signal specific to a disease or disorder) results in proteolytic release of multiple copies of phosphoprotein (7). At the same time, additional RNAs (8, 9) encoding nucleocapsid, large, matrix, and glycoprotein (or other viral ligand) are produced. Nucleocapsid and large (10) complex with released phosphoprotein to make copies of the complete viral genomic RNA (11), which supplies further templates for expression of all encoded proteins. Matrix and glycoprotein or other viral ligand (12) create evaginations of the membrane (13) which take up viral genomic RNAs, nucleocapsid, phosphoprotein, and large proteins and bud off as new virions.

According to some embodiments of the second aspects, the recombinant viral genome comprises a modified genome of a virus, where the virus is a vesicular stomatitis virus (VSV). When the recombinant viral genome comprises a modified genome of a VSV, in certain embodiments, the viral protein encoded by the deleted gene is VSV phosphoprotein (VSVP).

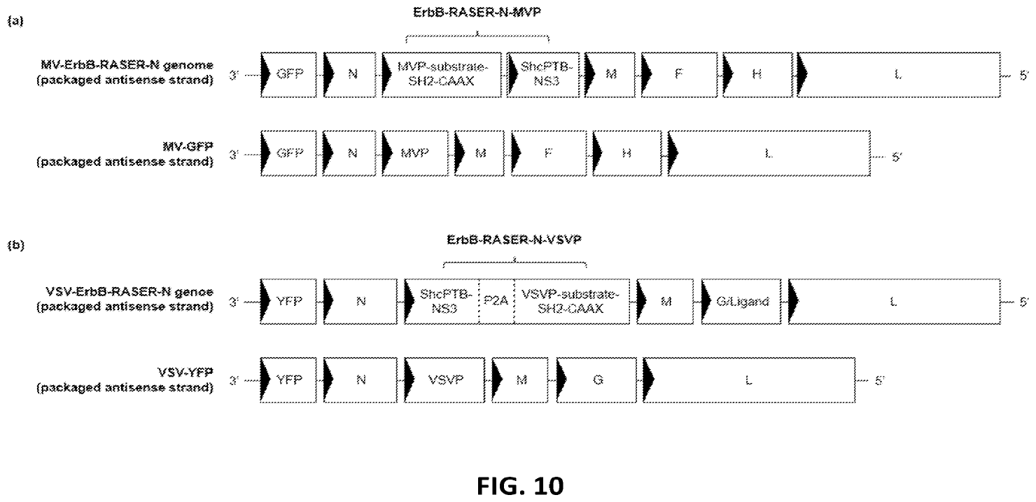

Non-limiting examples of recombinant viral genomes according to embodiments of the second aspects are schematically illustrated in FIG. 10. Shown in (a) is MV-ErbB-RASER-N, where each rectangle represents a transcription unit. Triangles denote transcription start sites recognized by the RNA-dependent polymerase complex of large, phosphoprotein, and nucleocapsid. Terminator sites recognized by this complex are at the end of each transcription unit. GFP, green fluorescent protein. N, nucleocapsid. M, matrix. F, fusion. H, hemagglutinin. L, large. MVP, MV phosphoprotein. A transcriptional unit expressing unfused MVP no longer exists, rendering virus replication conditional on MVP release from ErbB-RASER-N-MVP. Other arrangements of the coding sequences are possible, as is the addition of other coding sequences. Note the coding sequence for the fluorescent protein can be removed. Shown in (b) is VSV-ErbB-RASER-N, where each rectangle represents a transcription unit. Triangles denote transcription start sites recognized by the RNA-dependent polymerase complex of large, phosphoprotein, and nucleocapsid. Terminator sites recognized by this complex are at the end of each transcription unit. YFP, yellow fluorescent protein. N, nucleocapsid. M, matrix. G/Ligand, glycoprotein or other viral ligand. L, large. VSVP, VSV phosphoprotein. P2A, poliovirus ribosomal skipping element. A transcriptional unit expressing unfused MVP no longer exists, rendering virus replication conditional on MVP release from ErbB-RASER-N-MVP. The nucleotide sequences of the MV-ErbB-RASER-N and VSV-ErbB-RASER-N constructs, as well as the amino acid sequences of the first and second fusion proteins encoded by the constructs, are provided in Table 2 below.

TABLE 2
VSV-ErbB-RASER-N Nucleotide and Amino Acid Sequences

| | |
|---|---|
| MV-ErbB-RASER-N | accaaacaaagttgggtaaggatagttcaatcaatgatcatcttctagtgcacttaggattcaa |
| (SEQ ID NO: 6) | gatcctattatcagggacaagagcaggattagggatatcccgacgcgtacgccaccatggt |
| (reverse-complement of | gagcaagggcgaggagctgttcaccggggtggtgcccatcctggtcgagctggacggcga |
| RNA genome in DNA | cgtaaacggccacaagttcagcgtgtccggcgagggcgagggcgatgccacctacggca |
| form) | agctgaccctgaagttcatctgcaccaccggcaagctgcccgtgccctggcccaccctcgtg |
| | accaccctgacctacggcgtgcagtgcttcagccgctaccccgaccacatgaagcagcac |
| | gacttcttcaagtccgccatgcccgaaggctacgtccaggagcgcaccatcttcttcaaggac |
| | gacggcaactacaagacccgcgccgaggtgaagttcgagggcgacaccctggtgaaccg |
| | catcgagctgaagggcatcgacttcaaggaggacggcaacatcctggggcacaagctgg |
| | agtacaactacaacagccacaacgtctatatcatggccgacaagcagaagaacggcatca |
| | aggtgaacttcaagatccgccacaacatcgaggacggcagcgtgcagctcgccgaccact |
| | accagcagaacacccccatcggcgacggccccgtgctgctgcccgacaaccactacctga |
| | gcacccagtccgccctgagcaaagaccccaacgagaagcgcgatcacatggtcctgctgg |
| | agttcgtgaccgccgccgggatcactctcggcatggacgagctgtacaagtagactgacgtc |
| | tcgcgatcgcgtccgcctaccctccatcattgttataaaaaacttaggaaccaggtccacaca |
| | gccgccagcccatcaaccatccactcccacgattggagatatccgagatggccacactttta |
| | aggagcttagcattgttcaaaagaaacaaggacaaaccacccattacatcaggatccggtg |
| | gagccatcagaggaatcaaacacattattatagtaccaatccctggagattcctcaattacca |
| | ctcgatccagacttctggaccggttggtcaggttaattggaaacccggatgtgagcgggccc |
| | aaactaacaggggcactaataggtatattatccttatttgtggagtctccaggtcaattgattca |
| | gaggatcaccgatgaccctgacgttagcataaggctgttagaggttgtccagagtgaccagt |
| | cacaatctggccttaccttcgcatcaagaggtaccaacatggaggatgaggcggaccaata |
| | cttttcacatgatgatccaattagtagtgatcaatccaggttcggatggttcgagaacaaggaa |
| | atctcagatattgaagtgcaagaccctgagggattcaacatgattctgggtaccatcctagcc |
| | caaatttgggtcttgctcgcaaaggcggttacggccccagacacggcagctgattcggagct |
| | aagaaggtggataaagtacacccaacaaagaagggtagttggtgaatttagattggagag |
| | aaaatggttggatgtggtgaggaacaggattgccgaggacctctccttacgccgattcatggt |
| | cgctctaatcctggacatcaagagaacacccggaaacaaacccaggattgctgaaatgat |
| | atgtgacattgatacatatatcgtagaggcaggattagccagttttatcctgactattaagtttgg |
| | gatagaaactatgtatcctgctcttggactgcatgaatttgctggtgagttatccacacttgagtc |
| | cttgatgaacctttaccagcaaatgggggaaactgcaccctacatggtaatcctggagaact |
| | caattcagaacaagttcagtgcaggatcataccctctgctctggagctatgccatgggagtag |
| | gagtggaacttgaaaactccatgggaggtttgaactttggccgatcttactttgatccagcatatt |
| | ttagattagggcaagagatggtaaggaggtcagctggaaaggtcagttccacattggcatct |
| | gaactcggtatcactgccgaggatgcaaggcttgtttcagagattgcaatgcatactactgag |
| | gacaagatcagtagagcggttggacccagacaagcccaagtatcatttctacacggtgatc |
| | aaagtgagaatgagctaccgagattggggggcaaggaagataggagggtcaaacagag |
| | tcgaggagaagccagggagagctacagagaaaccgggcccagcagagcaagtgatgc |
| | gagagctgcccatcttccaaccggcacacccctagacattgacactgcatcggagtccagc |
| | caagatccgcaggacagtcgaaggtcagctgacgccctgcttaggctgcaagccatggca |
| | ggaatctcggaagaacaaggctcagacacggacacccctatagtgtacaatgacagaaat |
| | cttctagactaggtgcgagaggccgaggaccagaacaacatccgcctaccctccatcattgt |
| | tataaaaaacttaggaaccaggtccacacagccgccagcccatcaaccatccactcccac |
| | gattggagcagatggcagaagagcaggcacgccatgtcaaaaacggactggaatgcatc |
| | cgggctctcaaggccgagcccatcggctcactggccatcgaggaagctatggcagcatggt |
| | cagaaatatcagacaacccaggacaggagcgagccacctgcagggaagagaaggcag |
| | gcagttcgggtctcagcaaaccatgcctctcagcaattggatcaactgaaggcggtgcacct |
| | cgcatccgcggtcagggacctggagagagcgatgacgacgctgaaactttgggaatcccc |
| | ccaagaaatctccaggcatcaagcactgggctacagtgtcattatgtttatgatcacagcggt |
| | gaagcggttaagggaatccaagatgctgactctatcatggttcaatcaggccttgatggtgat |
| | agcaccctctcaggaggagacaatgaatctgaaaacagcgatgtggatattggcgaacctg |
| | ataccgagggatatgctatcactgaccggggatctgctcccatctctatggggttcagggcttct |
| | gatgttgaaactgcagaaggaggggagatccacgagctcctgagactccaatccagaggc |
| | aacaactttccgaagcttgggaaaactctcaatgttcctccgcccccggaccccggtagggc |
| | cagcacttccgggacacccattaaaaagggcacagacgcgagattagcctcatttggaacg |
| | gagatcgcgtcttcattgacaggtggtgcaacccaatgtgctcgaaagtcaccctcggaacc |
| | atcagggccaggtgcacctgcggggaacgtccccgagtgtgtgagcaatgccgcactgata |
| | caggagtggacacccgaatctggcaccacaatctccccgagatcccagaataatgaagaa |
| | gggggagaccattatgatgatgagctgttctctgatgtccaagatattaaaacagccttggcca |
| | aaatacacgaggataatcagaagataatctccaagctagaatcactgctgttattgaaggga |
| | gaagttgagtcaattaagaagcagatcaacaggcaaaatatcagcatatccaccctggaag |
| | gacacctctcaagcatcatgatcgccattcctggacttgggaaggatcccaacgaccccact |
| | gcagatgtcgaaatcaatcccgacttgaaacccatcataggcagagattcaggccgagcac |
| | tggccgaagttctcaagaaacccgttgccagccgacaactccaaggaatgacaaatggac |
| | ggaccagttccagaggacagctgctgaaggaatttcagctaaagccgatcgggaaaaaga |
| | tgagctcagccgtcgggtttgttcctgacaccggccctgcatcacgcagtgtaatccgctccatt |
| | ataaaatccagccggctagaggaggatcggaagcgttacctgatgactctccttgatgatatc |
| | aaaggagccaatgatcttgccaagttccaccagatgctgatgaagataataatgaaggatg |
| | agatggaagagtgctctaagcacctcggcggttcgggaggttctggtgggtcaggtggcagt |
| | ggcaagccaatggcgggcaacttcgactcggaggagcggagtagctggtactgggggcg |
| | gttgagtcggcaggaggcggtggcgctgctgcagggccagcggcacggggtgttcctggtg |
| | cgggactcgagcaccagccccggggactatgtgctcagcgtctcagagaactcgcgcgtct |
| | cccactacatcatcaacagcagcggcccgcgcccgccggtgccaccgtcgcccgcccag |
| | cctccgcccggggtgagcccctccagactccgaataggagatcaagagtttgattcattgcct |
| | gctttactggaattctacaaaatacactatttggacactacaacgttgatagaaccagttcttaa |
| | gggatctagaggtaaaccaatccccaatcctctgctgggtcttgatagcaccgaattcaagat |
| | gagcaaggacggcaagaagaaaaagaagaagtccaagactaagtgcgtgatcatgtgat |
| | gcgcatctacagctcaacttacctgccaaccccatgccagtcgacccaactagtacaaccta |
| | aatccattataaaaaacttaggagcaaagtgattgcctcccaagttccacattcgaaaccatg |
| | ggcaagccactgcatcccaacgacaaagtcatgggacccggggtttcctacttggttcggta |
| | catgggttgtgtggaggtcctccagtcaatgcgtgccctggacttcaacacccggactcaggt |
| | caccagggaggccatcagtctggtgtgtgaggctgtgccgggtgctaagggggcgacaag |
| | gaggagaaagccctgtagccgcccgctcagctctatcctggggaggagtaacctgaaattt |
| | gctggaatgccaatcactctcaccgtctccaccagcagcctcaacctcatggccgcagactg |
| | caaacagatcatcgccaaccaccacatgcaatctatctcatttgcatccggggggatccgg |
| | acacagccgagtatgtcgcctatgttgccaaagaccctgtgaatcagagagcctgccacatt |
| | ctggagtgtcccgaagggcttgcccaggatgtcatcagcaccattggccaggccttcgagttg |
| | cgcttcaaacaatacctcagggacatcgagcaggtgccccagcagcccacccttaagatg |
| | gattacaaggatgacgacgataagggctcttccgggacaggctccggatccggcactagtg |
| | cgcccatcacggcgtacgcccagcagacgagaggcctcctagggtgtataatcaccagcct |
| | gactggccgggacaaaaaccaagtggagggtgaggtccagatcgtgtcaactgctaccca |
| | aaccttcctggcaacgtgcatcaatggggtatgctggactgtctaccacggggccggaacga |
| | ggaccatcgcatcacccaagggtcctgtcatccagatgtataccaatgtggaccaagacctt |
| | gtgggctggcccgctcctcaaggttcccgctcattgacaccctgtacctgcggctcctcggac |
| | ctttacctggtcacgaggcacgccgatgtcattcccgtgcgccggcgaggtgatagcagggg |
| | tagcctgctttcgccccggcccatttcctacttgaaaggctcctcggggggtccgctgttgtgcc |
| | ccgcgggacacgccgtgggcctattcagggccgcggtgtgcacccgtggagtggctaaag |
| | cggtggactttatccctgtggagaacctagagacaaccatgagatagggcgcgccttgacta |
| | cagctcaacttacctgccaaccccatgccagtcgacccaactagtacaacctaaatccattat |
| | aaaaaacttaggagcaaagtgattgcctcccaagttccacaatgacagagatctacgacttc |
| | gacaagtcggcatgggacatcaaagggtcgatcgctccgatacaacccaccacctacagt |
| | gatggcaggctggtgccccaggtcagagtcatagatcctggtctaggcgacaggaaggatg |
| | aatgctttatgtacatgtttctgctgggggttgttgaggacagcgattccctagggcctccaatcg |
| | ggcgagcatttgggtccctgcccttaggtgttggcagatccacagcaaagcccgaaaaactc |
| | ctcaaagaggccactgagcttgacatagttgttagacgtacagcagggctcaatgaaaaact |
| | ggtgttctacaacaacaccccactaactctcctcacaccttggagaaaggtcctaacaacag |
| | ggagtgtcttcaacgcaaaccaagtgtgcaatgcggttaatctgataccgctcgataccccgc |
| | agaggttccgtgttgtttatatgagcatcacccgtctttcggataacgggtattacaccgttcctg |
| | gaagaatgctggaattcagatcggtcaatgcagtggccttcaacctgctggtgacccttagga |
| | ttgacaaggcgataggccctgggaagatcatcgacaatacagagcaacttcctgaggcaa |
| | catttatggtccacatcgggaacttcaggagaaagaagagtgaagtctactctgccgattattg |
| | caaaatgaaaatcgaaaagatgggcctggtttttgcacttggtgggatagggggcaccagtc |
| | ttcacattagaagcacaggcaaaatgagcaagactctccatgcacaactcgggttcaagaa |
| | gaccttatgttacccgctgatggatatcaatgaagaccttaatcgattactctggaggagcaga |
| | tgcaagatagtaagaatccaggcagttttgcagccatcagttcctcaagaattccgcatttacg |
| | acgacgtgatcataaatgatgaccaaggactattcaaagttctgtagaccgtagtgcccagc |
| | aatgcccgaaaacgacccccctcacaatgacagccagaaggcccggacaaaaaagccc |
| | cctccgaaagactccacggaccaagcgagaggccagccagcagccgacggcaagcgc |
| | gaacaccaggcggccccagcacagaacagccctgacacaaggccaccaccagccacc |
| | ccaatctgcatcctcctcgtgggacccccgaggaccaacccccaaggctgcccccgatcca |
| | aaccaccaaccgcatccccaccacccccgggaaagaaacccccagcaattggaaggcc |
| | cctccccctcttcctcaacacaagaactccacaaccgaaccgcacaagcgaccgaggtga |
| | cccaaccgcaggcatccgactccctagacagatcctctctccccggcaaactaaacaaaa |
| | cttagggccaaggaacatacacacccaacagaacccagaccccggcccacggcgccgc |
| | gcccccaacccccgacaaccagagggagcccccaaccaatcccgccggctcccccggt |
| | gcccacaggcagggacaccaacccccgaacagacccagcacccaaccatcgacaatcc |
| | aagacgggggggcccccccaaaaaaaggcccccaggggccgacagccagcaccgcg |
| | aggaagcccacccaccccacacacgaccacggcaaccaaaccagaacccagaccacc |
| | ctgggccaccagctcccagactcggccatcaccccgcagaaaggaaaggccacaaccc |
| | gcgcaccccagccccgatccggggggagccacccaacccgaaccagcacccaagag |
| | cgatccccgaaggacccccgaaccgcaaaggacatcagtatcccacagcctctccaagtc |
| | ccccggtctcctcctcttctcgaagggaccaaaagatcaatccaccacacccgacgacactc |
| | aactccccacccctaaaggagacaccgggaatcccagaatcaagactcatccaatgtccat |
| | catgggtctcaaggtgaacgtctctgccatattcatggcagtactgttaactctccaaacaccc |
| | accggtcaaatccattggggcaatctctctaagataggggggtaggaataggaagtgcaa |
| | gctacaaagttatgactcgttccagccatcaatcattagtcataaaattaatgcccaatataact |
| | ctcctcaataactgcacgagggtagagattgcagaatacaggagactactgagaacagtttt |
| | ggaaccaattagagatgcacttaatgcagtgacccagaatataagaccggttcagagtgta |
| | gcttcaagtaggagacacaagagatttgcgggagtagtcctggcaggtgcggccctaggcg |
| | ttgccacagctgctcagataacagccggcattgcacttcaccagtccatgctgaactctcaag |
| | ccatcgacaatctgagagcgagcctggaaactactaatcaggcaattgaggcaatcagac |
| | aagcagggcaggagatgatattggctgttcagggtgtccaagactacatcaataatgagctg |
| | ataccgtctatgaaccaactatcttgtgatttaatcggccagaagctcgggctcaaattgctca |
| | gatactatacagaaatcctgtcattatttggccccagcttacgggaccccatatctgcggagat |
| | atctatccaggctttgagctatgcgcttggaggagacatcaataaggtgttagaaaagctcgg |
| | atacagtggaggtgatttactgggcatcttagagagcagaggaataaaggcccggataact |
| | cacgtcgacacagagtcctacttcattgtcctcagtatagcctatccgacgctgtccgagatta |
| | agggggtgattgtccaccggctagagggggtctcgtacaacataggctctcaagagtggtat |
| | accactgtgcccaagtatgttgcaacccaagggtaccttatctcgaattttgatgagtcatcgtg |
| | tactttcatgccagaggggactgtgtgcagccaaaatgccttgtacccgatgagtcctctgctc |
| | caagaatgcctccgggggtccaccaagtcctgtgctcgtacactcgtatccgggtcttttggga |
| | accggttcattttatcacaagggaacctaatagccaattgtgcatcaatcctttgcaagtgttac |
| | acaacaggaacgatcattaatcaagaccctgacaagatcctaacatacattgctgccgatca |
| | ctgcccggtagtcgaggtgaacggcgtgaccatccaagtcgggagcaggaggtatccaga |
| | cgctgtgtacttgcacagaattgacctcggtcctcccatatcattggagaggttggacgtaggg |
| | acaaatctggggaatgcaattgctaagttggaggatgccaaggaattgttggagtcatcgga |
| | ccagatattgaggagtatgaaaggtttatcgagcactagcatagtctacatcctgattgcagtg |
| | tgtcttggagggttgatagggatccccgctttaatatgttgctgcagggggcgttgtaacaaaa |
| | agggagaacaagttggtatgtcaagaccaggcctaaagcctgatcttacgggaacatcaaa |
| | atcctatgtaaggtcgctctgatcctctacaactcttgaaacacaaatgtcccacaagtctcctc |
| | ttcgtcatcaagcaaccaccgcacccagcatcaagcccacctgaaattatctccggtttccaa |
| | gcggaagaacaatatcggtagttaattaaaacttagggtgcaagatcatccacaatgtcacc |
| | acaacgagaccggataaatgccttctacaaagataacccccatcccaagggaagtaggat |
| | agtcattaacagagaacatcttatgattgatagaccttatgttttgctggctgttctgtttgtcatgt |
| | ttctgagcttgatcgggttgctagccattgcaggcattagacttcatcgggcagccatctacaccg |
| | cagagatccataaaagcctcagcaccaatctagatgtaactaactcaatcgagcatcaggtc |
| | aaggacgtgctgacaccactcttcaaaatcatcggtgatgaagtgggcctgaggacacctc |
| | agagattcactgacctagtgaaattcatctctgacaagattaaattccttaatccggataggga |
| | gtacgacttcagagatctcacttggtgtatcaacccgccagagagaatcaaattggattatgat |
| | caatactgtgcagatgtggctgctgaagagctcatgaatgcattggtgaactcaactctactgg |
| | agaccagaacaaccaatcagttcctagctgtctcaaagggaaactgctcagggcccactac |
| | aatcagaggtcaattctcaaacatgtcgctgtccctgttagacttgtatttaggtcgaggttacaa |
| | tgtgtcatctatagtcactatgacatcccagggaatgtatgggggaacttacctagtggaaaa |
| | gcctaatctgagcagcaaaaggtcagagttgtcacaactgagcatgtaccgagtgtttgaagt |
| | aggtgttatcagaaatccgggtttgggggctccggtgttccatatgacaaactatcttgagcaa |
| | ccagtcagtaatgatctcagcaactgtatggtggctttgggggagctcaaactcgcagcccttt |
| | gtcacggggaagattctatcacaattccctatcagggatcagggaaaggtgtcagcttccag |
| | ctcgtcaagctaggtgtctggaaatccccaaccgacatgcaatcctgggtccccttatcaacg |
| | gatgatccagtgatagacaggctttacctctcatctcacagaggtgttatcgctgacaaccaa |
| | gcaaaatgggctgtcccgacaacacgaacagatgacaagttgcgaatggagacatgcttc |
| | caacaggcgtgtaagggtaaaatccaagcactctgcgagaatcccgagtgggcaccattg |
| | aaggataacaggattccttcatacggggtcttgtctgttgatctgagtctgacagttgagcttaa |
| | aatcaaaattgcttcgggattcgggccattgatcacacacggttcagggatggacctatacaa |
| | atccaaccacaacaatgtgtattggctgactatcccgccaatgaagaacctagccttaggtgt |
| | aatcaacacattggagtggataccgagattcaaggttagtccctacctcttcactgtcccaatta |
| | aggaagcaggcggagactgccatgccccaacatacctacctgcggaggtggatggtgatg |
| | tcaaactcagttccaatctggtgattctacctggtcaagatctccaatatgttttggcaacctacg |
| | atacttccagggttgaacatgctgtggtttattacgtttacagcccaagccgctcattttcttacttt |
| | tatccttttaggttgcctataaagggggtccccatcgaattacaagtggaatgcttcacatggga |
| | ccaaaaactctggtgccgtcacttctgtgtgcttgcggactcagaatctggtggacatatcactc |
| | actctgggatggtgggcatgggagtcagctgcacagtcacccgggaagatggaaccaatc |
| | gcagatagggctgctagtgaaccaatcacatgatgtcacccagacatcaggcatacccact |
| | agtgtgaaatagacatcagaattaagaaaaacgtagggtccaagtggttccccgttatggac |
| | tcgctatctgtcaaccagatcttataccctgaagttcacctagatagcccgatagttaccaataa |
| | gatagtagccatcctggagtatgctcgagtccctcacgcttacagcctggaggaccctacact |
| | gtgtcagaacatcaagcaccgcctaaaaaacggattttccaaccaaatgattataaacaatg |
| | tggaagttgggaatgtcatcaagtccaagcttaggagttatccggcccactctcatattccatat |
| | ccaaattgtaatcaggatttatttaacatagaagacaaagagtcaacgaggaagatccgtga |
| | actcctcaaaaaggggaattcgctgtactccaaagtcagtgataaggttttccaatgcttaagg |
| | gacactaactcacggcttggcctaggctccgaattgagggaggacatcaaggagaaagtta |
| | ttaacttgggagtttacatgcacagctcccagtggtttgagccctttctgttttggtttacagtcaag |
| | actgagatgaggtcagtgattaaatcacaaacccatacttgccataggaggagacacacac |
| | ctgtattcttcactggtagttcagttgagttgctaatctctcgtgaccttgttgctataatcagtaaa |
| | gagtctcaacatgtatattacctgacatttgaactggttttgatgtattgtgatgtcatagaggggag |
| | gttaatgacagagaccgctatgactattgatgctaggtatacagagcttctaggaagagtcag |
| | atacatgtggaaactgatagatggtttcttccctgcactcgggaatccaacttatcaaattgtag |
| | ccatgctggagcctctttcacttgcttacctgcagctgagggatataacagtagaactcagag |
| | gtgctttccttaaccactgctttactgaaatacatgatgttcttgaccaaaacgggttttctgatga |
| | aggtacttatcatgagttaattgaagctctagattacattttcataactgatgacatacatctgac |
| | aggggagattttctcatttttcagaagtttcggccaccccagacttgaagcagtaacggctgct |
| | gaaaatgttaggaaatacatgaatcagcctaaagtcattgtgtatgagactctgatgaaaggt |
| | catgccatattttgtggaatcataatcaacggctatcgtgacaggcacggaggcagttggcca |
| | ccgctgaccctccccctgcatgctgcagacacaatccggaatgctcaagcttcaggtgatgg |
| | gttaacacatgagcagtgcgttgataactggaaatcttttgctggagtgaaatttggctgctttat |
| | gcctcttagcctggatagtgatctgacaatgtacctaaaggacaaggcacttgctgctctccaa |
| | agggaatgggattcagtttacccgaaagagttcctgcgttacgaccctcccaagggaaccg |
| | ggtcacggaggcttgtagatgttttccttaatgattcgagctttgacccatatgatgtgataatgta |
| | tgttgtaagtggagcttacctccatgaccctgagttcaacctgtcttacagcctgaaagaaaag |
| | gagatcaaggaaacaggtagactttttgctaaaatgacttacaaaatgagggcatgccaagt |
| | gattgctgaaaatctaatctcaaacgggattggcaaatattttaaggacaatgggatggccaa |
| | ggatgagcacgatttgactaaggcactccacactctagctgtctcaggagtccccaaagatct |
| | caaagaaagtcacaggggggggccagtcttaaaaacctactcccgaagcccagtccaca |
| | caagtaccaggaacgtgagagcagcaaaagggtttatagggttccctcaagtaattcggca |
| | ggaccaagacactgatcatccggagaatatggaagcttacgagacagtcagtgcatttatca |
| | cgactgatctcaagaagtactgccttaattggagatatgagaccatcagcttgtttgcacagag |
| | gctaaatgagatttacggattgccctcatttttccagtggctgcataagaggcttgagacctctgt |
| | cctgtatgtaagtgaccctcattgcccccccgaccttgacgcccatatcccgttatataaagtcc |
| | ccaatgatcaaatcttcattaagtaccctatgggaggtatagaagggtattgtcagaagctgtg |
| | gaccatcagcaccattccctatctatacctggctgcttatgagagcggagtaaggattgcttcgt |
| | tagtgcaaggggacaatcagaccatagccgtaacaaaaagggtacccagcacatggccc |
| | tacaaccttaagaaacgggaagctgctagagtaactagagattactttgtaattcttaggcaa |
| | aggctacatgatattggccatcacctcaaggcaaatgagacaattgtttcatcacatttttttgtct |
| | attcaaaaggaatatattatgatgggctacttgtgtcccaatcactcaagagcatcgcaagatg |
| | tgtattctggtcagagactatagttgatgaaacaagggcagcatgcagtaatattgctacaac |
| | aatggctaaaagcatcgagagaggttatgaccgttaccttgcatattccctgaacgtcctaaa |
| | agtgatacagcaaattctgatctctcttggcttcacaatcaattcaaccatgacccgggatgta |
| | gtcatacccctcctcacaaacaacgacctcttaataaggatggcactgttgcccgctcctattg |
| | gggggatgaattatctgaatatgagcaggctgtttgtcagaaacatcggtgatccagtaacat |
| | catcaattgctgatctcaagagaatgattctcgcctcactaatgcctgaagagaccctccatca |
| | ggtaatgacacaacaaccgggggactcttcattcctagactgggctagcgacccttactcag |
| | caaatcttgtatgtgtccagagcatcactagactcctcaagaacataactgcaaggtttgtcct |
| | gatccatagtccaaacccaatgttaaaaggattattccatgatgacagtaaagaagaggac |
| | gagggactggcggcattcctcatggacaggcatattatagtacctagggcagctcatgaaat |
| | cctggatcatagtgtcacaggggcaagagagtctattgcaggcatgctggataccacaaaa |
| | ggcttgattcgagccagcatgaggaaggggggtttaacctctcgagtgataaccagattgtcc |
| | aattatgactatgaacaattcagagcagggatggtgctattgacaggaagaaagagaaatg |
| | tcctcattgacaaagagtcatgttcagtgcagctggcgagagctctaagaagccatatgtggg |
| | cgaggctagctcgaggacggcctatttacggccttgaggtccctgatgtactagaatctatgc |
| | gaggccaccttattcggcgtcatgagacatgtgtcatctgcgagtgtggatcagtcaactacg |
| | gatggttttttgtcccctcgggttgccaactggatgatattgacaaggaaacatcatccttgaga |
| | gtcccatatattggttctaccactgatgagagaacagacatgaagcttgccttcgtaagagcc |
| | ccaagtcgatccttgcgatctgctgttagaatagcaacagtgtactcatgggcttacggtgatg |
| | atgatagctcttggaacgaagcctggttgttggctaggcaaagggccaatgtgagcctggag |
| | gagctaagggtgatcactcccatctcaacttcgactaatttagcgcataggttgagggatcgta |
| | gcactcaagtgaaatactcaggtacatcccttgtccgagtggcgaggtataccacaatctcca |
| | acgacaatctctcatttgtcatatcagataagaaggttgatactaactttatataccaacaagga |
| | atgcttctagggttgggtgttttagaaacattgtttcgactcgagaaagataccggatcatctaa |
| | cacggtattacatcttcacgtcgaaacagattgttgcgtgatcccgatgatagatcatcccagg |
| | atacccagctcccgcaagctagagctgagggcagagctatgtaccaacccattgatatatga |
| | taatgcacctttaattgacagagatgcaacaaggctatacacccagagccataggaggcac |
| | cttgtggaatttgttacatggtccacaccccaactatatcacattttagctaagtccacagcacta |
| | tctatgattgacctggtaacaaaatttgagaaggaccatatgaatgaaatttcagctctcatag |
| | gggatgacgatatcaatagtttcataactgagtttctgctcatagagccaagattattcactatct |
| | acttgggccagtgtgcggccatcaattgggcatttgatgtacattatcatagaccatcagggaa |
| | atatcagatgggtgagctgttgtcatcgttcctttctagaatgagcaaaggagtgtttaaggtgct |
| | tgtcaatgctctaagccacccaaagatctacaagaaattctggcattgtggtattatagagcct |
| | atccatggtccttcacttgatgctcaaaacttgcacacaactgtgtgcaacatggtttacacatg |
| | ctatatgacctacctcgacctgttgttgaatgaagagttagaagagttcacatttctcttgtgtgaa |
| | agcgacgaggatgtagtaccggacagattcgacaacatccaggcaaaacacttatgtgttct |
| | ggcagatttgtactgtcaaccagggacctgcccaccaattcaaggtctaagaccggtagag |
| | aaatgtgcagttctaaccgaccatatcaaggcagaggctatgttatctccagcaggatcttcgt |
| | ggaacataaatccaattattgtagaccattactcatgctccctgacttatctccggcgaggatcg |
| | atcaaacagataagattgagagttgatccaggattcattttcgacgccctcgctgaggtaaatg |
| | tcagtcagccaaagatcggcagcaacaacatctcaaatatgagcatcaaggctttcagacc |
| | cccacacgatgatgttgcaaaattgctcaaagatatcaacacaagcaagcacaatcttccca |
| | tttcagggggcaatctcgccaattatgaaatccatgctttccgcagaatcgggttgaactcatct |
| | gcttgctacaaagctgttgagatatcaacattaattaggagatgccttgagccaggggagga |
| | cggcttgttcttgggtgagggatcgggttctatgttgatcacttataaggagatacttaaactaag |
| | caagtgcttctataatagtggggtttccgccaattctagatctggtcaaagggaattagcaccct |
| | atccctccgaagttggccttgtcgaacacagaatgggagtaggtaatattgtcaaagtgctcttt |
| | aacgggaggcccgaagtcacgtgggtaggcagtgtagattgcttcaatttcatagttagtaat |
| | atccctacctctagtgtggggtttatccattcagatatagagaccttgcctgacaaagatactat |
| | agagaagctagaggaattggcagccatcttatcgatggctctgctcctgggcaaaataggat |
| | caatactggtgattaagcttatgcctttcagcggggattttgttcagggatttataagttatgtagg |
| | gtctcattatagagaagtgaaccttgtataccctagatacagcaacttcatatctactgaatctta |
| | tttggttatgacagatctcaaggctaaccggctaatgaatcctgaaaagattaagcagcagat |
| | aattgaatcatctgtgaggacttcacctggacttataggtcacatcctatccattaagcaactaa |
| | gctgcatacaagcaattgtgggagacgcagttagtagaggtgatatcaatcctactctgaaa |
| | aaacttacacctatagagcaggtgctgatcaattgcgggttggcaattaacggacctaagctg |
| | tgcaaagaattgatccaccatgatgttgcctcagggcaagatggattgcttaattctatactcat |
| | cctctacagggagttggcaagattcaaagacaaccaaagaagtcaacaagggatgttcca |
| | cgcttaccccgtattggtaagtagcaggcaacgagaacttatatctaggatcacccgcaaatt |
| | ttgggggcacattcttctttactccgggaacagaaagttgataaataagtttatccagaatctca |
| | agtccggctatctgatactagacttacaccagaatatcttcgttaagaatctatccaagtcaga |
| | gaaacagattattatgacggggggtttgaaacgtgagtgggtttttaaggtaacagtcaagga |
| | gaccaaagaatggtataagttagtcggatacagtgccctgattaaggactaattggttgaact |
| | ccggaaccctaatcctgccctaggtggttaggcattatttgcaatatattaaagaaaactttgaa |
| | aatacgaagtttctattcccagctttgtctggt |
| MV-ErbB-RASER-N | MAEEQARHVKNGLECIRALKAEPIGSLAIEEAMAAWSEISDNPGQ |
| (SEQ ID NO: 7) | ERATCREEKAGSSGLSKPCLSAIGSTEGGAPRIRGQGPGESDDD |
| MVP-substrate-SH2- | AETLGIPPRNLQASSTGLQCHYVYDHSGEAVKGIQDADSIMVQSG |
| CAAX | LDGDSTLSGGDNESENSDVDIGEPDTEGYAITDRGSAPISMGFRA |
| (encoded by nucleotides | SDVETAEGGEIHELLRLQSRGNNFPKLGKTLNVPPPPDPGRASTS |
| 2659 to 4735 of the | GTPIKKGTDARLASFGTEIASSLTGGATQCARKSPSEPSGPGAPA |
| above | GNVPECVSNAALIQEWTPESGTTISPRSQNNEEGGDHYDDELFS |
| MV-ErbB-RASER-N | DVQDIKTALAKIHEDNQKIISKLESLLLLKGEVESIKKQINRQNISIST |
| nucleotide sequence) | LEGHLSSIMIAIPGLGKDPNDPTADVEINPDLKPIIGRDSGRALAEVL |
| | KKPVASRQLQGMTNGRTSSRGQLLKEFQLKPIGKKMSSAVGFVP |
| | DTGPASRSVIRSIIKSSRLEEDRKRYLMTLLDDIKGANDLAKFHQM |
| | LMKIIMKEDVVCCSMSYGGSGGSGGSGGSGKPMAGNFDSEERS |
| | SWYWGRLSRQEAVALLQGQRHGVFLVRDSSTSPGDYVLSVSEN |
| | SRVSHYIINSSGPRPPVPPSPAQPPPGVSPSRLRIGDQEFDSLPAL |
| | LEFYKIHYLDTTTLIEPVLKGSRGKPIPNPLLGLDSTEFKMSKDGKK |
| | KKKKSKTKCVIM |
| MV-ErbB-RASER-N | MGKPLHPNDKVMGPGVSYLVRYMGCVEVLQSMRALDFNTRTQV |
| (SEQ ID NO: 8) | TREAISLVCEAVPGAKGATRRRKPCSRPLSSILGRSNLKFAGMPIT |
| ShcPTB-NS3 | LTVSTSSLNLMAADCKQIIANHHMQSISFASGGDPDTAEYVAYVAK |
| (encoded by nucleotides | DPVNQRACHILECPEGLAQDVISTIGQAFELRFKQYLRDIEQVPQQ |
| 4858 to 6018 of the | PTLKMDYKDDDDKGSSGTGSGSGTSAPITAYAQQTRGLLGCIITS |
| above | LTGRDKNQVEGEVQIVSTATQTFLATCINGVCWTVYHGAGTRTIA |
| MV-ErbB-RASER-N | SPKGPVIQMYTNVDQDLVGWPAPQGSRSLTPCTCGSSDLYLVTR |
| nucleotide sequence) | HADVIPVRRRGDSRGSLLSPRPISYLKGSSGGPLLCPAGHAVGLF |
| | RAAVCTRGVAKAVDFIPVENLETTMR |
| VSV-ErbB-RASER-N | acgaagacaaacaaaccattattatcattaaaaggctcaggagaaactttaacagtaatca |
| (SEQ ID NO: 9) | gaattctcgaggatccaccggtcgccaccatggtgagcaagggcgaggagctgttcaccgg |
| | ggtggtgcccatcctggtcgagctggacggcgacgtaaacggccacaagttcagcgtgtcc |
| | ggcgagggcgagggcgatgccacctacggcaagctgaccctgaagttaatctgcaccacc |
| | ggcaagctgcccgtgccctggcccaccctcgtgaccaccttaggctacggcctgcagtgctt |
| | cgcccgctaccccgaccacatgaagcagcacgacttcttcaagtccgccatgcccgaagg |
| | ctacgtccaggagcgcaccatcttcttcaaggacgacggcaactacaagacccgcgccga |
| | ggtgaagttcgagggcgacaccctggtgaaccgcatcgagctgaagggcatcgacttcaa |
| | ggaggacggcaacatcctggggcacaagctggagtacaactacaacagccacaacgtct |
| | atatcacggccgacaagcagaagaacggcatcaaggcgaacttcaagatccgccacaac |
| | atcgaggacggcggcgtgcagctcgccgaccactaccagcagaacacccccatcggcga |
| | cggccccgtgctgctgcccgacaaccactacctgagctaccagtccgccctgagcaaaga |
| | ccccaacgagaagcgcgatcacatggtcctgctggagttcgtgaccgccgccgggatcact |
| | ctcggcatggacgagctgtacaagtaaagcggccgccctcctcaggtgctggccatatgaa |
| | aaaaactaacagtaatcaaaatgtctgttacagtcaagagaatcattgacaacacagtcata |
| | gttccaaaacttcctgcaaatgaggatccagtggaatacccggcagattacttcagaaaatc |
| | aaaggagattcctctttacatcaatactacaaaaagtttgtcagatctaagaggatatgtctacc |
| | aaggcctcaaatccggaaatgtatcaatcatacatgtcaacagctacttgtatggagcattaa |
| | aggacatccggggtaagttggataaagattggtcaagtttcggaataaacatcgggaaagc |
| | aggggatacaatcggaatatttgaccttgtatccttgaaagccctggacggcgtacttccagat |
| | ggagtatcggatgcttccagaaccagcgcagatgacaaatggttgcctttgtatctacttggctt |
| | atacagagtgggcagaacacaaatgcctgaatacagaaaaaagctcatggatgggctga |
| | caaatcaatgcaaaatgatcaatgaacagtttgaacctcttgtgccagaaggtcgtgacattttt |
| | gatgtgtggggaaatgacagtaattacacaaaaattgtcgctgcagtggacatgttcttccac |
| | atgttcaaaaaacatgaatgtgcctcgttcagatacggaactattgtttccagattcaaagattg |
| | tgctgcattggcaacatttggacacctctgcaaaataaccggaatgtctacagaagatgtaac |
| | gacctggatcttgaaccgagaagttgcagatgaaatggtccaaatgatgcttccaggccaag |
| | aaattgacaaggccgattcatacatgccttatttgatcgactttggattgtcttctaagtctccatat |
| | tcttccgtcaaaaaccctgccttccacttctgggggcaattgacagctcttctgctcagatccac |
| | cagagcaaggaatgcccgacagcctgatgacattgagtatacatctcttactacagcaggttt |
| | gttgtacgcttatgcagtaggatcctctgccgacttggcacaacagttttgtgttggagataaca |
| | aatacactccagatgatagtaccggaggattgacgactaatgcaccgccacaaggcagag |
| | atgtggtcgaatggctcggatggtttgaagatcaaaacagaaaaccgactcctgatatgatg |
| | cagtatgcgaaaagagcagtcatgtcactgcaaggcctaagagagaagacaattggcaa |
| | gtatgctaagtcagaatttgacaaatgaccctataattctcagatcacctattatatattatgctac |
| | atatgaaaaaaactaacagatatcatgggcaagccactgcatcccaacgacaaagtcatg |
| | ggacccggggtttcctacttggttcggtacatgggttgtgtggaggtcctccagtcaatgcgtgc |
| | cctggacttcaacacccggactcaggtcaccagggaggccatcagtctggtgtgtgaggctg |
| | tgccgggtgctaagggggcgacaaggaggagaaagccctgtagccgcccgctcagctcta |
| | tcctggggaggagtaacctgaaatttgctggaatgccaatcactctcaccgtctccaccagca |
| | gcctcaacctcatggccgcagactgcaaacagatcatcgccaaccaccacatgcaatctat |
| | ctcatttgcatccggcatgttagctccctatatcccagagtatgtcgcctatgttgccaaagacc |
| | ctgtgaatcagagagcctgccacattctggagtgtcccgaagggcttgcccaggatgtcatca |
| | gcaccattggccaggccttcgagttgcgcttcaaacaatacctcagggacatcgagcaggtg |
| | ccccagcagcccacccttaagatggattacaaggatgacgacgataagggctcttccggga |
| | caggctccggatccggcactagtgcgcccatcacggcgtacgcccagcagacgagaggc |
| | ctcctagggtgtataatcaccagcctgactggccgggacaaaaaccaagtggagggtgag |
| | gtccagatcgtgtcaactgctacccaaaccttcctggcaacgtgcatcaatggggtatgctgg |
| | actgtctaccacggggccggaacgaggaccatcgcatcacccaagggtcctgtcatccag |
| | atgtataccaatgtggaccaagaccttgtgggctggcccgctcctcaaggttcccgctcattga |
| | caccctgtacctgcggctcctcggacctttacctggtcacgagGCACGCCGATGTCA |
| | TTCCCGTGCGCCGGCGAGGtgatagcaggggtagcctgctttcgccccggccc |
| | atttcctacttgaaaggctcctcggggggtccgctgttgtgccccgcgggacacgccgtgggc |
| | ctattcagggccgcggtgtgcacccgtggagtggctaaagcggtggactttatccctgtggag |
| | aacctagagacaaccatgagatccccggtgttctcggacaattcctccccaccagcggtgg |
| | gctctggagccaccaacttcagcctgctgaagcaggcaggcgacgtggaagagaaccctg |
| | gccctatggataatctcacaaaagttcgtgagtatctcaagtcctactctcgtctagatcaggcg |
| | gtaggagagatagatgagatcgaagcacaacgagctgaaaagtccaattatgagttgttcc |
| | aagaggacggagtggaagagcatactaggccctcttattttcaggcagcagatgattctgac |
| | acagaatctgaaccagaaattgaagacaatcaaggcttgtatgtaccagatccggaagctg |
| | agcaagttgaaggctttatacaggggcctttagatgactatgcagatgaggacgtggatgttgt |
| | attcacttcggactggaaacagcctgagcttgaatccgacgagcatggaaagaccttacggt |
| | tgacattgccagagggtttaagtggagagcagaaatcccagtggcttttgacgattaaagca |
| | gtcgttcaaagtgccaaacactggaatctggcagagtgcacatttgaagcatcgggagaag |
| | gggtcatcataaaaaagcgccagataactccggatgtatataaggtcactccagtgatgaac |
| | acacatccgtaccaatcagaagccgtatcagatgtttggtctctctcaaagacatccatgacttt |
| | ccaacccaagaaagcaagtcttcagcctctcaccatatccttggatgaattgttctcatctaga |
| | ggagaattcatctctgtcggaggtaacggacgaatgtctcataaagaggccatcctgctcggt |
| | ctgaggtacaaaaagttgtacaatcaggcgagagtcaaatattctctgggtaaaccaatccc |
| | caatcctctgctgggtcttgatagcaccgatgagatggaagagtgctctcagcacctccccgg |
| | gggtagcgggggttctggcggttcgggaggttctaagcctttttctacacaagacaccgacct |
| | cgacttggaaatgctcgctccatatatccctatggaggaggaatttcaactcagatcatttgac |
| | cagctctccccaaaaaagaaaggcggttcgggaggttctggtgggtcaggtggcagtggcg |
| | gccctcctcaggacctgtctgttcatctctggtacgcaggccccatggagcgggcaggggca |
| | gagagcatcctggccaaccgctcggacgggactttcttggtgcggcagagggtgaaggatg |
| | cagcagaatttgccatcagcattaaatataacgtcgaggtcaagcacattaaaatcatgaca |
| | gcagaaggactgtaccggatcacagagaaaaaggctttccgggggcttacggagctggtg |
| | gagttttaccagcagaactctctaaaggattgcttcaagtctctggacaccaccttgcagttccc |
| | cttcaaggagcctgaaaagagaggatctagagggggatctggtggatcagggggtagcgg |
| | gggttctggcggacaattcaagatgagcaaggacggcaagaagaaaaagaagaagtcc |
| | aagactaagtgcgtgatcatgtgaactatgaaaaaaagtaacagatatcacaatctaagtgtt |
| | atcccaatccattcatcatgagttccttaaagaagattctcggtctgaaggggaaaggtaaga |
| | aatctaagaaattagggatcgcaccacccccttatgaagaggacactagcatggagtatgct |
| | ccgagcgctccaattgacaaatcctattttggagttgacgagatggacacctatgatccgaat |
| | caattaagatatgagaaattcttctttacagtgaaaatgacggttagatctaatcgtccgttcag |
| | aacatactcagatgtggcagccgctgtatcccattgggatcacatgtacatcggaatggcag |
| | ggaaacgtcccttctacaaaatcttggcttttttgggttcttctaatctaaaggccactccagcggt |
| | attggcagatcaaggtcaaccagagtatcacgctcactgcgaaggcagggcttatttgccac |
| | ataggatggggaagacccctcccatgctcaatgtaccagagcacttcagaagaccattcaat |
| | ataggtctttacaagggaacgattgagctcacaatgaccatctacgatgatgagtcactggaa |
| | gcagctcctatgatctgggatcatttcaattcttccaaattttctgatttcagagagaaggccttaa |
| | tgtttggcctgattgtcgagaaaaaggcatctggagcgtgggtcctggattctatcagccacttc |
| | aaatgagctagtctagcttccagcttctgaacaatccccggtttactcagtctctcctaattccag |
| | cctttcgaacaactaatatcctgtcttttctatccctatgaaaaaaactaacagagatcgatctgt |
| | ttccttgacacgcgtcacgatgaagtgccttttgtacttaGcctttttattcattggggtgaattgca |
| | agttcaccatagtttttccacacaaccaaaaaggaaactggaaaaatgttccttctaattacca |
| | ttattgcccgtcaagctcagatttaaattggcataatgacttaataggcacagccttacaagtca |
| | aaatgcccaagagtcacaaggctattcaagcagacggttggatgtgtcatgcttccaaatgg |
| | gtcactacttgtgatttccgctggtatggaccgaagtatataacacattccatccgatccttcact |
| | ccatctgtagaacaatgcaaggaaagcattgaacaaacgaaacaaggaacttggctgaat |
| | ccaggcttccctcctcaaagttgtggatatgcaactgtgacggatgccgaagcagtgattgtcc |
| | aggtgactcctcaccatgtgctggttgatgaatacacaggagaatgggttgattcacagttcat |
| | caacggaaaatgcagcaattacatatgccccactgtccataactctacaacctggcattctga |
| | ctataaggtcaaagggctatgtgattctaacctcatttccatggacatcaccttcttctcagagg |
| | acggagagctatcatccctgggaaaggagggcacagggttcagaagtaactactttgcttat |
| | gaaactggaggcaaggcctgcaaaatgcaatactgcaagcattggggagtcagactccca |
| | tcaggtgtctggttcgagatggctgataaggatctctttgctgcagccagattccctgaatgccc |
| | agaagggtcaagtatctctgctccatctcagacctcagtggatgtaagtctaattcaggacgtt |
| | gagaggatcttggattattccctctgccaagaaacctggagcaaaatcagagcgggtcttcc |
| | aatctctccagtggatctcagctatcttgctcctaaaaacccaggaaccggtcctgctttcacca |
| | taatcaatggtaccctaaaatactttgagaccagatacatcagagtcgatattgctgctccaat |
| | cctctcaagaatggtcggaatgatcagtggaactaccacagaaagggaactgtgggatgac |
| | tgggcaccatatgaagacgtggaaattggacccaatggagttctgaggaccagttcaggat |
| | ataagtttcctttatacatgattggacatggtatgttggactccgatcttcatcttagctcaaaggct |
| | caggtgttcgaacatcctcacattcaagacgctgcttcgcaacttcctgatgatgagagtttattt |
| | tttggtgatactgggctatccaaaaatccaatcgagcttgtagaaggttggttcagtagttggaa |
| | aagctctattgcctcttttttctttatcatagggttaatcattggactattcttggttctccgagttg |
| | gtatccatctttgcattaaattaaagcacaccaagaaaagacagatttatacagacatagagatga |
| | accgacttggaaagtaactcgcggccgccctgcacaacagattcttcatgtttgaaccaaatc |
| | aacttgtgatatcatgctcaaagaggccttaattatattttaatttttaatttttatgaaaaaaacta |
| | acagcaatcatggaagtccacgattttgagaccgacgagttcaatgatttcaatgaagatgact |
| | atgccacaagagaattcctgaatcccgatgagcgcatgacgtacttgaatcatgctgattaca |
| | atttgaattctcctctaattagtgatgatattgacaatttgatcaggaaattcaattctcttccgatt |
| | ccctcgatgtgggatagtaagaactgggatggagttcttgagatgttaacatcatgtcaagcca |
| | atcccatctcaacatctcagatgcataaatggatgggaagttggttaatgtctgataatcatgat |
| | gccagtcaagggtatagttttttacatgaagtggacaaagaggcagaaataacatttgacgtg |
| | gtggagaccttcatccgcggctggggcaacaaaccaattgaatacatcaaaaaggaaaga |
| | tggactgactcattcaaaattctcgcttatttgtgtcaaaagtttttggacttacacaagttgacatt |
| | aatcttaaatgctgtctctgaggtggaattgctcaacttggcgaggactttcaaaggcaaagtc |
| | agaagaagttctcatggaacgaacatatgcaggattagggttcccagcttgggtcctacttttat |
| | ttcagaaggatgggcttacttcaagaaacttgatattctaatggaccgaaactttctgttaatggt |
| | caaagatgtgattatagggaggatgcaaacggtgctatccatggtatgtagaatagacaacc |
| | tgttctcagagcaagacatcttctcccttctaaatatctacagaattggagataaaattgtggag |
| | aggcagggaaatttttcttatgacttgattaaaatggtggaaccgatatgcaacttgaagctgat |
| | gaaattagcaagagaatcaaggcctttagtcccacaattccctcattttgaaaatcatatcaag |
| | acttctgttgatgaaggggcaaaaattgaccgaggtataagattcctccatgatcagataatg |
| | agtgtgaaaacagtggatctcacactggtgatttatggatcgttcagacattggggtcatcctttt |
| | atagattattacactggactagaaaaattacattcccaagtaaccatgaagaaagatattgat |
| | gtgtcatatgcaaaagcacttgcaagtgatttagctcggattgttctatttcaacagttcaatgatc |
| | ataaaaagtggttcgtgaatggagacttgctccctcatgatcatccctttaaaagtcatgttaaa |
| | gaaaatacatggcccacagctgctcaagttcaagattttggagataaatggcatgaacttccg |
| | ctgattaaatgttttgaaatacccgacttactagacccatcgataatatactctgacaaaagtca |
| | ttcaatgaataggtcagaggtgttgaaacatgtccgaatgaatccgaacactcctatccctagt |
| | aaaaaggtgttgcagactatgttggacacaaaggctaccaattggaaagaatttcttaaaga |
| | gattgatgagaagggcttagatgatgatgatctaattattggtcttaaaggaaaggagaggga |
| | actgaagttggcaggtagatttttctccctaatgtcttggaaattgcgagaatactttgtaattacc |
| | gaatatttgataaagactcatttcgtccctatgtttaaaggcctgacaatggcggacgatctaac |
| | tgcagtcattaaaaagatgttagattcctcatccggccaaggattgaagtcatatgaggcaatt |
| | tgcatagccaatcacattgattacgaaaaatggaataaccaccaaaggaagttatcaaacg |
| | gcccagtgttccgagttatgggccagttcttaggttatccatccttaatcgagagaactcatgaa |
| | ttttttgagaaaagtcttatatactacaatggaagaccagacttgatgcgtgttcacaacaacac |
| | actgatcaattcaacctcccaacgagtttgttggcaaggacaagagggtggactggaaggtc |
| | tacggcaaaaaggatggagtatcctcaatctactggttattcaaagagaggctaaaatcaga |
| | aacactgctgtcaaagtcttggcacaaggtgataatcaagttatttgcacacagtataaaacg |
| | aagaaatcgagaaacgttgtagaattacagggtgctctcaatcaaatggtttctaataatgag |
| | aaaattatgactgcaatcaaaatagggacagggaagttaggacttttgataaatgacgatga |
| | gactatgcaatctgcagattacttgaattatggaaaaataccgattttccgtggagtgattagag |
| | ggttagagaccaagagatggtcacgagtgacttgtgtcaccaatgaccaaatacccacttgt |
| | gctaatataatgagctcagtttccacaaatgctctcaccgtagctcattttgctgagaacccaat |
| | caatgccatgatacagtacaattattttgggacatttgctagactcttgttgatgatgcatgatcct |
| | gctcttcgtcaatcattgtatgaagttcaagataagataccgggcttgcacagttctactttcaaa |
| | tacgccatgttgtatttggacccttccattggaggagtgtcgggcatgtctttgtccaggtttttgat |
| | tagagccttcccagatcccgtaacagaaagtctctcattctggagattcatccatgtacatgctc |
| | gaagtgagcatctgaaggagatgagtgcagtatttggaaaccccgagatagccaagtttcg |
| | aataactcacatagacaagctagtagaagatccaacctctctgaacatcgctatgggaatga |
| | gtccagcgaacttgttaaagactgaggttaaaaaatgcttaatcgaatcaagacaaaccatc |
| | aggaaccaggtgattaaggatgcaaccatatatttgtatcatgaagaggatcggctcagaag |
| | tttcttatggtcaataaatcctctgttccctagatttttaagtgaattcaaatcaggcacttttttgg |
| | gagtcgcagacgggctcatcagtctatttcaaaattctcgtactattcggaactcctttaagaaaaa |
| | gtatcatagggaattggatgatttgattgtgaggagtgaggtatcctctttgacacatttaggga |
| | aacttcatttgagaaggggatcatgtaaaatgtggacatgttcagctactcatgctgacacatt |
| | aagatacaaatcctggggccgtacagttattgggacaactgtaccccatccattagaaatgtt |
| | gggtccacaacatcgaaaagagactccttgtgcaccatgtaacacatcagggttcaattatgt |
| | ttctgtgcattgtccagacgggatccatgacgtctttagttcacggggaccattgcctgcttatcta |
| | gggtctaaaacatctgaatctacatctattttgcagccttgggaaagggaaagcaaagtccca |
| | ctgattaaaagagctacacgtcttagagatgctatctcttggtttgttgaacccgactctaaacta |
| | gcaatgactatactttctaacatccactctttaacaggcgaagaatggaccaaaaggcagca |
| | tgggttcaaaagaacagggtctgcccttcataggttttcgacatctcggatgagccatggtggg |
| | ttcgcatctcagagcactgcagcattgaccaggttgatggcaactacagacaccatgaggg |
| | atctgggagatcagaatttcgactttttattccaagcaacgttgctctatgctcaaattaccacca |
| | ctgttgcaagagacggatggatcaccagttgtacagatcattatcatattgcctgtaagtcctgtt |
| | tgagacccatagaagagatcaccctggactcaagtatggactacacgcccccagatgtatc |
| | ccatgtgctgaagacatggaggaatggggaaggttcgtggggacaagagataaaacagat |
| | ctatcctttagaagggaattggaagaatttagcacctgctgagcaatcctatcaagtcggcag |
| | atgtataggttttctatatggagacttggcgtatagaaaatctactcatgccgaggacagttctct |
| | atttcctctatctatacaaggtcgtattagaggtcgaggtttcttaaaagggttgctagacggatt |
| | aatgagagcaagttgctgccaagtaatacaccggagaagtctggctcatttgaagaggccg |
| | gccaacgcagtgtacggaggtttgatttacttgattgataaattgagtgtatcacctccattccttt |
| | ctcttactagatcaggacctattagagacgaattagaaacgattccccacaagatcccaacct |
| | cctatccgacaagcaaccgtgatatgggggtgattgtcagaaattacttcaaataccaatgcc |
| | gtctaattgaaaagggaaaatacagatcacattattcacaattatggttattctcagatgtcttat |
| | ccatagacttcattggaccattctctatttccaccaccctcttgcaaatcctatacaagccattttt |
| | atctgggaaagataagaatgagttgagagagctggcaaatctttcttcattgctaagatcagga |
| | gagggggggaagacatacatgtgaaattcttcaccaaggacatattattgtgtccagagga |
| | aatcagacatgcttgcaagttcgggattgctaaggataataataaagacatgagctatccccc |
| | ttggggaagggaatccagagggacaattacaacaatccctgtttattatacgaccacccctta |
| | cccaaagatgctagagatgcctccaagaatccaaaatcccctgctgtccggaatcaggttgg |
| | gccaattaccaactggcgctcattataaaattcggagtatattacatggaatgggaatccatta |
| | cagggacttcttgagttgtggagacggctccggagggatgactgctgcattactacgagaaa |
| | atgtgcatagcagaggaatattcaatagtctgttagaattatcagggtcagtcatgcgaggcg |
| | cctctcctgagccccccagtgccctagaaactttaggaggagataaatcgagatgtgtaaat |
| | ggtgaaacatgttgggaatatccatctgacttatgtgacccaaggacttgggactatttcctccg |
| | actcaaagcaggcttggggcttcaaattgatttaattgtaatggatatggaagttcgggattcttc |
| | tactagcctgaaaattgagacgaatgttagaaattatgtgcaccggattttggatgagcaagg |
| | agttttaatctacaagacttatggaacatatatttgtgagagcgaaaagaatgcagtaacaatc |
| | cttggtcccatgttcaagacggtcgacttagttcaaacagaatttagtagttctcaaacgtctga |
| | agtatatatggtatgtaaaggtttgaagaaattaatcgatgaacccaatcccgattggtcttcca |
| | tcaatgaatcctggaaaaacctgtacgcattccagtcatcagaacaggaatttgccagagca |
| | aagaaggttagtacatactttaccttgacaggtattccctcccaattcattcctgatccttttgtaa |
| | acattgagactatgctacaaatattcggagtacccacgggtgtgtctcatgcggctgccttaaa |
| | atcatctgatagacctgcagatttattgaccattagccttttttatatggcgattatatcgtattat |
| | aacatcaatcatatcagagtaggaccgatacctccgaaccccccatcagatggaattgcacaa |
| | aatgtggggatcgctataactggtataagcttttggctgagtttgatggagaaagacattccact |
| | atatcaacagtgtttggcagttatccagcaatcatttccgattaggtgggaggctatttcagtaa |
| | aaggaggatacaagcagaagtggagtactagaggtgatgggctcccaaaagatacccga |
| | atttcagactccttggccccaatcgggaactggatcagatctttggaattggtccgaaaccaa |
| | gttcgtctaaatccattcaataagatcttgttcaatcagctatgtcgtacagtggataatcatttga |
| | agtggtcaaatttgcgaaaaaacacaggaatgattgaatggatcaatgggcgaatttcaaaa |
| | gaagaccggtctatactgatgttgaagagtgacctacatgaggaaaactcttggagagatta |
| | aaaaatcaggaggagactccaaactttaagtatgaaaaaaactttgatccttaagaccctctt |
| | gtggtttttatttttttatctggttttgtggtcttcgtgggtcggcatggcatctccacctcctcgcg |
| | gtccgacctgggcatccgaaggaggacgtcgtccactcggatggctaagggagagctcggatc |
| | cggctgctaacaaagcccgaaaggaagctgagttggctgctgccaccgctgagcaataact |
| | agcataaccccttggggcctctaaacgggtcttgaggggttttttgctgaaaggaggaactata |
| | tccggatcgagatcctctagagtcgacctgcaggcatgcaagcttgtattctatagtgtcaccta |
| | aatcgtatgtgtatgatacataaggttatgtattaattgtagccgcgttctaacgacaatatgtac |
| | aagcctaattgtgtagcatctggcttactgaagcagaccctatcatctctctcgtaaactgccgt |
| | cagagtcggtttggttggacgaaccttctgagtttctggtaacgccgtcccgcacccggaaatg |
| | gtcagcgaaccaatcagcagggtcatcgctagccagatcctctacgccggacgcatcgtgg |
| | ccggcatcaccggcgccacaggtgcggttgctggcgcctatatcgccgacatcaccgatgg |
| | ggaagatcgggctcgccacttcgggctcatgagcgcttgtttcggcgtgggtatggtggcagg |
| | ccccgtggccgggggactgttgggcgccatctccttgcaccattccttgcggcggcggtgctc |
| | aacggcctcaacctactactgggctgcttcctaatgcaggagtcgcataagggagagcgtcg |
| | aatggtgcactctcagtacaatctgctctgatgccgcatagttaagccagccccgacacccgc |
| | caacacccgctgacgcgccctgacgggcttgtctgctcccggcatccgcttacagacaagct |
| | gtgaccgtctccgggagctgcatgtgtcagaggttttcaccgtcatcaccgaaacgcgcgag |
| | acgaaagggcctcgtgatacgcctatttttataggttaatgtcatgataataatggtttcttagac |
| | gtcaggtggcacttttcggggaaatgtgcgcggaacccctatttgtttatttttctaaatacattca |
| | aatatgtatccgctcatgagacaataaccctgataaatgcttcaataatattgaaaaaggaag |
| | agtatgagtattcaacatttccgtgtcgcccttattcccttttttgcggcattttgccttcctgttt |
| | ttgctcacccagaaacgctggtgaaagtaaaagatgctgaagatcagttgggtgcacgagtgggtt |
| | acatcgaactggatctcaacagcggtaagatccttgagagttttcgccccgaagaacgttttcc |
| | aatgatgagcacttttaaagttctgctatgtggcgcggtattatcccgtattgacgccgggcaag |
| | agcaactcggtcgccgcatacactattctcagaatgacttggttgagtactcaccagtcacag |
| | aaaagcatcttacggatggcatgacagtaagagaattatgcagtgctgccataaccatgagt |
| | gataacactgcggccaacttacttctgacaacgatcggaggaccgaaggagctaaccgcttt |
| | tttgcacaacatgggggatcatgtaactcgccttgatcgttgggaaccggagctgaatgaagc |
| | cataccaaacgacgagcgtgacaccacgatgcctgtagcaatggcaacaacgttgcgcaa |
| | actattaactggcgaactacttactctagcttcccggcaacaattaatagactggatggaggc |
| | ggataaagttgcaggaccacttctgcgctcggcccttccggctggctggtttattgctgataaat |
| | ctggagccggtgagcgtgggtctcgcggtatcattgcagcactggggccagatggtaagcc |
| | ctcccgtatcgtagttatctacacgacggggagtcaggcaactatggatgaacgaaatagac |
| | agatcgctgagataggtgcctcactgattaagcattggtaactgtcagaccaagtttactcatat |
| | atactttagattgatttaaaacttcatttttaatttaaaaggatctaggtgaagatcctttttgataa |
| | tctcatgaccaaaatcccttaacgtgagttttcgttccactgagcgtcagaccccgtagaaaagat |
| | caaaggatcttcttgagatcctttttttctgcgcgtaatctgctgcttgcaaacaaaaaaaccacc |
| | gctaccagcggtggtttgtttgccggatcaagagctaccaactctttttccgaaggtaactggct |
| | tcagcagagcgcagataccaaatactgttcttctagtgtagccgtagttaggccaccacttcaa |
| | gaactctgtagcaccgcctacatacctcgctctgctaatcctgttaccagtggctgctgccagtg |
| | gcgataagtcgtgtcttaccgggttggactcaagacgatagttaccggataaggcgcagcgg |
| | tcgggctgaacggggggttcgtgcacacagcccagcttggagcgaacgacctacaccgaa |
| | ctgagatacctacagcgtgagctatgagaaagcgccacgcttcccgaagggagaaaggc |
| | ggacaggtatccggtaagcggcagggtcggaacaggagagcgcacgagggagcttcca |
| | gggggaaacgcctggtatctttatagtcctgtcgggtttcgccacctctgacttgagcgtcgattt |
| | ttgtgatgctcgtcaggggggcggagcctatggaaaaacgccagcaacgcggcctttttacg |
| | gttcctggccttttgctggccttttgctcacatgttctttcctgcgttatcccctgattctgtggata |
| | accgtattaccgcctttgagtgagctgataccgctcgccgcagccgaacgaccgagcgcagcg |
| | agtcagtgagcgaggaagcggaagagcgcccaatacgcaaaccgcctctccccgcgcgt |
| | tggccgattcattaatgcagggggatctcgatcccgcgaaattaatacgactcactatagg |
| VSV-ErbB-RASER-N | MDNLTKVREYLKSYSRLDQAVGEIDEIEAQRAEKSNYELFQEDGV |
| (SEQ ID NO: 10) | EEHTRPSYFQAADDSDTESEPEIEDNQGLYVPDPEAEQVEGFIQG |
| VSVP-NS3-substrate | PLDDYADEDVDVVFTSDWKQPELESDEHGKTLRLTLPEGLSGEQ |
| SH2-CAAX | KSQWLLTIKAVVQSAKHWNLAECTFEASGEGVIIKKRQITPDVYKV |
| (encoded by nucleotides | TPVMNTHPYQSEAVSDVWSLSKTSMTFQPKKASLQPLTISLDELF |
| 3463 to 4971 of the | SSRGEFISVGGNGRMSHKEAILLGLRYKKLYNQARVKYSLGKPIP |
| above | NPLLGLDSTDEMEECSQHLPGGSGGSGGSGGSKPFSTQDTDLD |
| VSV-ErbB-RASER-N | LEMLAPYIPMEEEFQLRSFDQLSPKKKGGSGGSGGSGGSGGPP |
| nucleotide sequence) | QDLSVHLWYAGPMERAGAESILANRSDGTFLVRQRVKDAAEFAIS |
| | IKYNVEVKHIKIMTAEGLYRITEKKAFRGLTELVEFYQQNSLKDCFK |
| | SLDTTLQFPFKEPEKRGSRGGSGGSGGSGGSGGQFKMSKDGKK |
| | KKKKSKTKCVIM |
| VSV-ErbB-RASER-N | MGKPLHPNDKVMGPGVSYLVRYMGCVEVLQSMRALDENTRTQV |
| (SEQ ID NO: 3) | TREAISLVCEAVPGAKGATRRRKPCSRPLSSILGRSNLKFAGMPIT |
| ShcPTB-NS3 | LTVSTSSLNLMAADCKQIIANHHMQSISFASGMLAPYIPEYVAYVA |
| (encoded by nucleotides | KDPVNQRACHILECPEGLAQDVISTIGQAFELRFKQYLRDIEQVPQ |
| 2197 to 3396 of the | QPTLKMDYKDDDDKGSSGTGSGSGTSAPITAYAQQTRGLLGCIIT |
| above | SLTGRDKNQVEGEVQIVSTATQTFLATCINGVCWTVYHGAGTRTI |
| VSV-ErbB-RASER-N | ASPKGPVIQMYTNVDQDLVGWPAPQGSRSLTPCTCGSSDLYLVT |
| nucleotide sequence) | RHADVIPVRRRGDSRGSLLSPRPISYLKGSSGGPLLCPAGHAVGL |
| | FRAAVCTRGVAKAVDFIPVENLETTMRSPVFSDNSSPPAV |

According to any of the embodiments of the first and second aspects, the first and second binding domains may bind to a variety of first and second targets in a variety of configurations. In certain embodiments, the first and second targets are present on the same or different proteins. That is, according to some embodiments, the first and second targets are present on the same protein. For example, the first and second targets may be different epitopes of the same protein. In certain other embodiments, the first and second targets are present on different proteins. When the first and second targets are present on different proteins, the different proteins may form a complex with each other. According to some embodiments, such a complex is present at a higher level in cancer cells than non-cancer cells.

In certain embodiments of the first and second aspects, the first and second targets are present on one or more proteins that are specifically expressed by cancer cells. According to some embodiments, the first and second targets are present on one or more proteins that are expressed at a higher level in cancer cells compared to non-cancer cells.

By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, a cancer cell line, and the like.

According to any of the embodiments of the first and second aspects, a variety of suitable first and second binding domains may be employed. In certain embodiments, one or both of the first and second binding domains comprise an antibody binding domain. The term “antibody” (also used interchangeably with “immunoglobulin”) encompasses antibodies of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the antigen, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information system®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, an antibody of the present disclosure comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments which may be genetically encoded or produced by digestion with various peptidases. Thus, for example, pepsin digests an antibody below the disulfide linkages in the hinge region to produce F(ab)′₂, a dimer of Fab which itself is a light chain joined to VH-CHI by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or synthesized de novo using recombinant DNA methodologies. In certain embodiments, one or both of the first and second binding domains comprise a single chain antibody, e.g., an scFv or the like.

According to any of the embodiments of the first and second aspects, one or both of the first and second binding domains comprise a binding domain of a protein, e.g., a protein other than an antibody. In one non-limiting example, one or both of the first and second binding domains comprise a phosphotyrosine binding domain. For example, one or both of the first and second binding domains may comprise a Src homology 2 (SH2) domain or a Shc domain, e.g., when it is desirable for one or both of the first and second binding domains to phosphotyrosines on an ErbB protein such as HER2, EGFR, or the like.

In certain embodiments according to any of the first and second aspects, the first and second targets are in proximity to each other such that the protease cleaves the cleavable substrate when the first and second binding domains are bound to their respective targets.

According to any of the embodiments of the first and second aspects, the first and second targets may be present on the same protein. When the first and second targets are present on the same protein, in some embodiments, the same protein is a receptor or a transmembrane protein. Receptors and transmembrane proteins of interest include, but are not limited to, stem cell receptors, immune cell receptors, growth factor receptors, cytokine receptors, hormone receptors, receptor tyrosine kinases, immune receptors such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type Il cytokine receptor such as interferon-alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2(1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, and ErbB protein (e.g., a hyperactive ErbB protein), HER2 (e.g., a hyperactive HER2 protein), EGFR (e.g., a hyperactive EGFR protein), a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, an eosinophil receptor, a chimeric antigen receptor (CAR), a recombinant T cell receptor (TCR), etc. When the same protein is a receptor or transmembrane protein (e.g., any of the proteins described above in the present paragraph), the receptor or transmembrane protein is hyperactive or signals constitutively.

In certain embodiments, when the first and second targets are present on the same protein, the same protein is hyperactive or signals constitutively. According to some embodiments, the same protein is a mutant protein. In certain embodiments, the same protein is human EGF receptor 2 (HER2). According to some embodiments, the HER2 protein is hyperactive or signals constitutively. In certain embodiments, the same protein is epidermal growth factor receptor (EGFR). According to some embodiments, the EGFR protein is hyperactive or signals constitutively. In certain embodiments, the first binding domain of the first fusion protein and the second binding domain of the second fusion protein is independently selected from a Src homology 2 (SH2) domain and a Shc domain.

The first fusion protein may be engineered to comprise any suitable protease of interest. In one non-limiting example, the first fusion protein comprises a viral protease. In certain embodiments, the first fusion protein comprises a hepatitis C virus (HCV) nonstructural protein 3 (NS3) protease. According to some embodiments, a HCV NS3 protease is derived from HCV NS3. NS3 consists of an N-terminal serine protease domain and a C-terminal helicase domain. By “derived from HCV NS3” is meant the protease comprises or consists of the serine protease domain of HCV NS3 or a proteolytically active variant thereof capable of cleaving a cleavage site for the serine protease domain of HCV NS3. The protease domain of NS3 forms a heterodimer with the HCV nonstructural protein 4A (NS4A), which activates proteolytic activity. A protease derived from HCV NS3 may include the entire NS3 protein or a proteolytically active fragment thereof, and may further include a cofactor polypeptide, such as a cofactor polypeptide derived from HCV nonstructural protein 4A (NS4A), e.g., an activating NS4A region. NS3 protease is highly selective and can be inhibited by a number of non-toxic, cell-permeable drugs, which are currently available for use in humans. NS3 protease inhibitors that may be employed include, but are not limited to, simeprevir, danoprevir, asunaprevir, ciluprevir, boceprevir, sovaprevir, paritaprevir, telaprevir, grazoprevir, and any combination thereof.

The second fusion protein may be engineered to comprise any suitable membrane targeting signal of interest. In certain embodiments, the membrane-targeting signal comprises a transmembrane domain. According to some embodiments, the membrane-targeting signal comprises a membrane-tethering domain. When the membrane-targeting signal comprises a membrane-tethering domain, in certain embodiments, the membrane-tethering domain comprises a post-translational modification. Non-limiting examples of post-translational modifications include palmitoylation, myristoylation, prenylation, a glycosylphosphatidylinositol (GPI) anchor, or any combination thereof.

The first fusion protein, the second fusion protein, or both, may comprise a protein domain that emits a detectable signal. For example, one or both of the first and second fusion proteins may comprise a bioluminescence reporter domain. In some embodiments, the bioluminescence reporter domain comprises a luciferase, e.g., a nanoluciferase, a firefly luciferase, or the like. One or both of the first and second fusion proteins may comprise a fluorescence reporter domain. In some embodiments, the fluorescence reporter domain comprises green fluorescent protein (GFP), red fluorescent protein (RFP), yellow fluorescent protein (YFP), or the like.

### Cells, Virions and Compositions

Also provided by the present disclosure are cells (e.g., host cells) comprising any of the recombinant viral genomes of the present disclosure, including any of the recombinant viral genomes described in the section above entitled “Recombinant Viral Genomes”. “Recombinant host cells,” “host cells,” “cells,” “cell lines,” “cell cultures,” and other such terms denoting microorganisms or higher eukaryotic cell lines, refer to cells which can be, or have been, used as recipients for a recombinant viral genome or other transferred DNA, and include the progeny of the cell which has been transfected. Host cells may be cultured as unicellular or multicellular entities (e.g., tissue, organs, or organoids) including an expression vector of the present disclosure. A recombinant viral genome may be present in any host of interest, e.g., mammalian cells, insect cells, plant cells, yeast, bacteria, and the like. In some embodiments, provided is a cell of a viral packaging cell line comprising any of the recombinant viral genomes of the present disclosure.

The present disclosure also provides virions produced by a cell of a viral packaging cell line comprising any of the recombinant viral genomes of the present disclosure. Virions comprising any of the recombinant viral genomes of the present disclosure are also provided.

Also provided by the present disclosure are compositions. A composition of the present disclosure may include any of the recombinant viral genomes of the present disclosure, any of the virions of the present disclosure, and/or the like. In some embodiment, a subject composition comprises a recombinant viral genome and/or a virion of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure comprises any of the virions of the present disclosure, where the pharmaceutical composition optionally comprises a pharmaceutically acceptable carrier.

The virions can be incorporated into a variety of formulations for therapeutic administration. More particularly, the virions can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the virions for administration to the individual (e.g., suitable for human administration) are generally sterile and may further be free of detectable pyrogens or other contaminants contraindicated for administration to a patient according to a selected route of administration. The following methods and carriers/excipients are merely examples and are in no way limiting.

The virions can be formulated for parenteral (e.g., intravenous, intra-arterial, intra-tumoral, intraosseous, intramuscular, intracerebral, intracerebroventricular, intrathecal, subcutaneous, etc.) administration. In certain embodiments, the virions are formulated for injection by dissolving, suspending or emulsifying the virions in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, the virions can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Pharmaceutical compositions that include the virions may be prepared by mixing the virions having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

An aqueous formulation of the virions may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the virions against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the virions and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods of Use

The present disclosure also provides methods of using the recombinant viral genomes, virions and/or pharmaceutical compositions of the present disclosure.

In some aspects, provided are methods of treating a disease or condition in a subject in need thereof, such methods comprising administering a pharmaceutical composition of the present disclosure to the subject. According to some embodiments, the pharmaceutical composition comprises the virions in an amount effective to induce death of cells exhibiting a signal specific to the disease or disorder in the subject. In certain embodiments, the pharmaceutical composition comprises the virions in an amount effective to induce death of cells exhibiting a signal that is higher than the signal in non-diseased cells.

According to the methods of treating a disease or condition in a subject in need thereof, in certain embodiments, the disease or condition is a cancer. The terms “cancer” and “cancerous” refer to or describe the physiological condition in mammals that is typically characterized by unregulated cell growth/proliferation. In some embodiments, the subject has a cancer characterized by the presence of a solid tumor, a semi-solid tumor, a primary tumor, a metastatic tumor, or the like. In some embodiments, the subject has a cancer selected from breast cancer (e.g., HER2+ breast cancer), melanoma, lung cancer, colorectal cancer, prostate cancer, glioma, gioblastoma, bladder cancer, endometrial cancer, kidney cancer, leukemia (e.g., acute myeloid leukemia (AML)), liver cancer (e.g., hepatocellular carcinoma (HCC), such as primary or recurrent HCC), non-Hodgkin lymphoma, pancreatic cancer, thyroid cancer, and any sub-types thereof. In certain embodiments, the cancer is selected from breast cancer, ovarian cancer, and non-small cell lung cancer.

By “treat”, “treating” or “treatment” is meant at least an amelioration of the symptoms associated with the disease or condition (e.g., cancer) of the individual, where amelioration is used in a broad sense to refer to at least a reduction in the magnitude of a parameter, e.g., symptom, associated with the disease or condition being treated. As such, treatment also includes situations where the disease or condition, or at least symptoms associated therewith, are completely inhibited, e.g., prevented from happening, or stopped, e.g., terminated, such that the individual no longer suffers from the disease or condition, or at least the symptoms that characterize the disease or condition.

In some embodiments, an effective amount of the virions (or pharmaceutical composition comprising same) is an amount that, when administered alone (e.g., in monotherapy) or in combination (e.g., in combination therapy) with one or more additional therapeutic agents, in one or more doses, is effective to reduce the symptoms of disease or condition of the subject (e.g., cancer, etc.) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the subject in the absence of treatment with the virions or pharmaceutical composition.

In certain embodiments, the disease or condition is characterized by hyperactive ErbB signaling, e.g., a cell proliferative disease or condition (e.g., cancer) characterized by hyperactive ErbB signaling. For example, the disease or condition characterized by hyperactive ErbB signaling may be a disease or condition (e.g., a cell proliferative disease or condition, e.g., cancer) characterized by hyperactive epidermal growth factor receptor (EGFR) signaling. Also by way of example, the disease or condition characterized by hyperactive ErbB signaling may be a disease or condition (e.g., a cell proliferative disease or condition, e.g., cancer) characterized by human EGF receptor 2 (HER2) signaling.

### Viral Production Methods

Also provided by the present disclosure are methods of producing viruses/virions. According to some embodiments, provided are methods of producing a conditionally replicating or nonreplicating virus of the order mononegavirus. Such methods comprise infecting a first mammalian cell population with a virus expressing a bacteriophage T7 polymerase. The first mammalian cell population comprises one or more plasmids expressing nucleocapsid, phosphoprotein, and large (polymerase) protein for a species of the virus of the order mononegavirus under the transcriptional control of T7 promoters. The first mammalian cell population further comprises a plasmid comprising a viral genome encoding the virus of the order mononegavirus lacking phosphoprotein or large protein, or encoding phosphoprotein or large protein in a conditionally functional form, under the transcriptional control of a T7 promoter. The methods further comprise culturing a second mammalian cell population with the infected first mammalian cell population and/or a culture medium thereof, wherein the second mammalian cell population stably expresses nucleocapsid, phosphoprotein, and large (polymerase) protein, under conditions in which the second mammalian cell population produces the virus of the order mononegavirus.

According to some embodiments, the virus of the order mononegavirus is VSV. In certain embodiments, the first mammalian cell population is a population of HEK293 cells or derivatives thereof. According to some embodiments, the first mammalian cell population is a population of HEK293T cells. In certain embodiments, the second mammalian cell population is a population of BHK21 cells. According to some embodiments, the second mammalian cell population is a population of Vero cells. In certain embodiments, the first mammalian cell population is a population of HEK293T cells, the second mammalian cell population is a population of BHK21 cells, and the virus of the order mononegavirus is VSV.

Notwithstanding the appended claims, the present disclosure is also defined by the following embodiments 1-86:

1. A recombinant viral genome comprising:
(a) a first transcription unit comprising:
   (i) a first sequence encoding a first fusion protein comprising a protease and a first binding domain that binds to a first target, and
   (ii) a second sequence encoding a second fusion protein comprising, in N-terminal to C-terminal order or in C-terminal to N-terminal order, a membrane-targeting signal, a second binding domain that binds to a second target, a substrate cleavable by the protease, and a transcriptional effector protein; and
(b) a second transcription unit comprising a third sequence encoding one or more viral replication proteins, wherein the second transcription unit is operably linked to a promoter activatable by the transcriptional effector protein.
   2. The recombinant viral genome of embodiment 1, wherein the recombinant viral genome comprises a modified genome of a virus.
   3. The recombinant viral genome of embodiment 2, wherein the virus is an oncolytic virus.
   4. The recombinant viral genome of any one of embodiments 1 to 3, wherein the promoter is not activatable by the transcriptional effector protein until the transcriptional effector protein is cleaved from the second fusion protein.
   5. The recombinant viral genome of any one of embodiments 1 to 3, wherein the promoter is activatable to a lesser extent by the second fusion protein compared to the transcriptional effector protein when cleaved from the second fusion protein.
   6. The recombinant viral genome of any one of embodiments 1 to 5, wherein the transcriptional effector protein comprises a transcription factor.
   7. The recombinant viral genome of any one of embodiments 2 to 6, wherein the one or more viral replication proteins encoded by the second transcription unit is required for replication of the virus in cells or efficient replication of the virus in cells.
   8. The recombinant viral genome of any one of embodiments 2 to 7, wherein replication of the virus in cells is dependent on a signal specific to cancer cells or a signal in cancer cells that is higher than the signal in non-cancer cells.
   9. The recombinant viral genome of any one of embodiments 2 to 8, wherein replication of the virus in cells is not dependent on the presence of an endogenous transcription factor.
   10. The recombinant viral genome of embodiment 9, wherein the endogenous transcription factor is an endogenous cancer-selective transcription factor.
   11. The recombinant viral genome of any one of embodiments 1 to 10, wherein the recombinant viral genome further comprises a third transcription unit encoding an immunostimulatory molecule, optionally wherein the immunostimulatory molecule is granulocyte/macrophage colony stimulating factor (GM-CSF).
   12. The recombinant viral genome of any one of embodiments 2 to 11, wherein the virus is a double-stranded DNA virus.
   13. The recombinant viral genome of embodiment 12, wherein the double-stranded DNA virus is an adenovirus.
   14. The recombinant viral genome of embodiment 13, wherein the one or more viral replication proteins comprise one or more E1 gene products.
   15. The recombinant viral genome of any one of embodiments 1 to 14, wherein the transcriptional effector protein comprises a GAL4-VP16 transcription factor.
   16. The recombinant viral genome of embodiment 15, wherein the promoter activatable by the transcriptional effector protein is a GAL4 UAS promoter.
   17. A recombinant viral genome, comprising:
(a) a deficiency in at least one native viral gene; and
(b) one or more transcription units comprising:
   (i) a sequence encoding a first fusion protein comprising a protease and a first binding domain that binds to a first target, and
   (ii) a sequence encoding a second fusion protein comprising, in N-terminal to C-terminal order or in C-terminal to N-terminal order, a membrane-targeting signal, a second binding domain that binds to a second target, a substrate cleavable by the protease, and the viral protein encoded by the deleted gene.
      18. The recombinant viral genome of embodiment 17, wherein the recombinant viral genome comprises a modified genome of a virus.
      19. The recombinant viral genome of embodiment 18, wherein the virus is a wild-type virus.
      20. The recombinant viral genome of embodiment 18, wherein the virus is an engineered virus.
      21. The recombinant viral genome of embodiment 18, wherein the virus is an RNA virus.
      22. The recombinant viral genome of embodiment 19, wherein the RNA virus is a negative-strand RNA virus.
      23. The recombinant viral genome of embodiment 22, wherein the negative-strand RNA virus is a mononegavirus.
      24. The recombinant viral genome of embodiment 23, wherein the mononegavirus is a rhabdovirus.
      25. The recombinant viral genome of embodiment 24, wherein the rhabdovirus is a vesicular stomatitis virus (VSV).
      26. The recombinant viral genome of embodiment 25, wherein the viral protein encoded by the deleted gene is VSV phosphoprotein (VSVP).
      27. The recombinant viral genome of any one of embodiments 1 to 26, wherein the first and second targets are present on the same protein.
      28. The recombinant viral genome of embodiment 27, wherein the first and second targets are different epitopes of the same protein.
      29. The recombinant viral genome of any one of embodiments 1 to 26, wherein the first and second targets are present on different proteins.
      30. The recombinant viral genome of embodiment 29, wherein the different proteins form a complex with each other.
      31. The recombinant viral genome of embodiment 30, wherein the complex is present at a higher level in cancer cells than non-cancer cells.
      32. The recombinant viral genome of any one of embodiments 1 to 28, wherein the first and second targets are present on one or more proteins that are specifically expressed by cancer cells.
      33. The recombinant viral genome of any one of embodiments 1 to 28, wherein the first and second targets are present on one or more proteins that are expressed at a higher level in cancer cells compared to non-cancer cells.
      34. The recombinant viral genome of any one of embodiments 1 to 28, 32, or 33, wherein the first binding domain is a phosphotyrosine binding domain.
      35. The recombinant viral genome of any one of embodiments 1 to 28, 32, or 33, wherein the second binding domain is a phosphotyrosine binding domain.
      36. The recombinant viral genome of any one of embodiments 1 to 28, 32, or 33, wherein the first binding domain is a phosphotyrosine binding domain and the second binding domain is a phosphotyrosine binding domain.
      37. The recombinant viral genome of any one of embodiments 1 to 36, wherein the first and second targets are in proximity to each other such that the protease cleaves the cleavable substrate when the first and second binding domains are bound to their respective targets.
      38. The recombinant viral genome of embodiment 27, wherein the same protein is a receptor or a transmembrane protein.
      39. The recombinant viral genome of embodiment 27, wherein the same protein is hyperactive or signals constitutively.
      40. The recombinant viral genome of embodiment 27, wherein the same protein is a mutant protein.
      41. The recombinant viral genome of embodiment 27, wherein the same protein is human EGF receptor 2 (HER2).
      42. The recombinant viral genome of embodiment 27, wherein the same protein is epidermal growth factor receptor (EGFR).
      43. The recombinant viral genome of any one of embodiments 1 to 42, wherein the first binding domain of the first fusion protein and the second binding domain of the second fusion protein is independently selected from a Src homology 2 (SH2) domain and a Shc domain.
      44. The recombinant viral genome of any one of embodiments 1 to 43, wherein the protease is a viral protease.
      45. The recombinant viral genome of embodiment 44, wherein the viral protease is a hepatitis C virus (HCV) nonstructural protein 3 (NS3) protease.
      46. The recombinant viral genome of any one of embodiments 1 to 45, wherein the membrane-targeting signal comprises a transmembrane domain.
      47. The recombinant viral genome of embodiment 46, wherein the membrane-targeting signal comprises a membrane-tethering domain.
      48. The recombinant viral genome of embodiment 47, wherein the membrane-tethering domain comprises a post-translational modification.
      49. The recombinant viral genome of embodiment 48, wherein the post-translational modification comprises palmitoylation, myristoylation, prenylation, a glycosylphosphatidylinositol (GPI) anchor, or any combination thereof.
      50. The recombinant viral genome of any one of embodiments 1 to 49, wherein the first fusion protein, the second fusion protein, or both, comprise a protein domain that emits a detectable signal.
      51. A cell comprising the recombinant viral genome of any one of embodiments 1 to 50.
      52. The cell of embodiment 51, wherein the cell is a mammalian cell.
      53. The cell of embodiment 51 or embodiment 52, wherein the cell is that of a viral packaging cell line.
      54. A virion produced by the cell of embodiment 53.
      55. A virion comprising the recombinant viral genome of any one of embodiments 1 to 50.
      56. A pharmaceutical composition comprising a plurality of virions of embodiment 55.
      57. A method of treating a disease or condition in a subject in need thereof comprising administering the pharmaceutical composition of embodiment 56 to the subject.
      58. The method of embodiment 57, wherein the pharmaceutical composition comprises the virions in an amount effective to induce death of cells exhibiting a signal specific to the disease or disorder in the subject.
      59. The method of embodiment 57, wherein the pharmaceutical composition comprises the virions in an amount effective to induce death of cells exhibiting a signal that is higher than the signal in non-diseased cells.
      60. The method of embodiment 57, wherein the disease or condition is a cancer.
      61. The method of embodiment 57, wherein the disease or condition is characterized by hyperactive ErbB signaling.
      62. The method according to embodiment 61, wherein the disease or condition characterized by hyperactive ErbB signaling is a disorder characterized by hyperactive epidermal growth factor receptor (EGFR) signaling.
      63. The method according to embodiment 61, wherein the disease or condition characterized by hyperactive ErbB signaling is a disease or condition characterized by hyperactive human EGF receptor 2 (HER2) signaling.
      64. The method according to embodiment 63, wherein the disease or condition characterized by hyperactive ErbB signaling is a cell proliferative disease or condition.
      65. The method according to embodiment 64, wherein the cell proliferative disease or condition is a cancer.
      66. The method according to embodiment 65, wherein the cancer is selected from breast cancer, ovarian cancer, and non-small cell lung cancer.
      67. A method of producing a conditionally replicating or nonreplicating mononegavirus, the method comprising:
(a) infecting a first cell population with a virus expressing a DNA sequence-specific RNA polymerase, wherein the first cell population comprises:
   (i) one or more plasmids containing one or more genes encoding a nucleocapsid, a phosphoprotein, and a large protein of a mononegavirus wherein the one or more genes are linked to a DNA sequence recognized by the RNA polymerase, and
   (ii) a plasmid comprising a genome of the mononegavirus linked to the DNA sequence recognized by the DNA sequence-specific RNA polymerase; and
(b) culturing a second cell population with the first cell population and/or a culture medium thereof, wherein the second cell population expresses a nucleocapsid, a phosphoprotein, and a large protein, under one or more conditions in which the second cell population produces the mononegavirus.
   68. The method according to embodiment 67, wherein the DNA sequence-specific RNA polymerase is a bacteriophage T7 RNA polymerase.
   69. The method according to embodiment 67, wherein the mononegavirus is a rhabdovirus.
   70. The method according to embodiment 69, wherein the rhabdovirus is a VSV.
   71. The method according to any one of embodiments 67 to 70, wherein the first cell population comprises HEK293T cells.
   72. The method according to any one of embodiments 67 to 71, wherein the second cell population comprises BHK21 cells.
   73. The method according to any one of embodiments 67 to 72, wherein the second cell population comprises Vero cells.
   74. The method according to any one of embodiments 67 to 73, wherein the nucleocapsid, the phosphoprotein, or the large protein, or a combination thereof, are conditionally functional.
   75. The method according to any one of embodiments 67 to 73, wherein the nucleocapsid, the phosphoprotein, or the large protein, or a combination thereof, are nonfunctional.
   76. A method of producing a conditionally replicating or nonreplicating virus, the method comprising:
(a) infecting a first cell population with a virus expressing an RNA polymerase, wherein the first cell population comprises:
   (i) one or more plasmids containing one or more genes encoding a nucleocapsid, a phosphoprotein, and a large protein of the virus to be produced, wherein the one or more genes are linked to a DNA sequence recognized by the RNA polymerase so that they can be transcribed by the RNA polymerase, and
   (ii) a plasmid comprising a genome of the virus to be produced wherein the viral genome is linked to the DNA sequence recognized by the RNA polymerase so that it can be transcribed by the RNA polymerase; and
(b) culturing a second cell population with the first cell population or a culture medium thereof, wherein the second cell population stably expresses the nucleocapsid, the phosphoprotein, and the large protein, under one or more conditions in which the second cell population produces the virus.
   77. The method according to embodiment 76, wherein the RNA polymerase is a bacteriophage T7 RNA polymerase.
   78. The method according to embodiment 76 or embodiment 77, wherein the virus is a mononegavirus.
   79. The method according to embodiment 78, wherein the mononegavirus is a rhabdovirus.
   80. The method according to embodiment 79, wherein the rhabdovirus is a VSV.
   81. The method according to any one of embodiments 78 to 80, wherein the viral genome of the mononegavirus encodes phosphoprotein in a conditionally functional form.
   82. The method according to any one of embodiments 76 to 81, wherein the first mammalian cell population comprises HEK293T cells.
   83. The method according to any one of embodiments 76 to 82, wherein the second cell population comprises BHK21 cells.
   84. The method according to any one of embodiments 76 to 83, wherein the second cell population comprises Vero cells.
   85. The method according to any one of embodiments 76 to 84, wherein the nucleocapsid, the phosphoprotein, or the large protein, or a combination thereof, are conditionally functional.
   86. The method according to any one of embodiments 76 to 84, wherein the nucleocapsid, the phosphoprotein, or the large protein, or a combination thereof, are nonfunctional.
   Notwithstanding the appended claims, the present disclosure is also defined by the following further embodiments B1-B40:
   B1. A recombinant viral genome, comprising:
a first transcription unit encoding:
   a first fusion protein comprising a phosphotyrosine-binding domain and a protease, and
   a second fusion protein comprising, in N-terminal to C-terminal order or in C-terminal to N-terminal order, a membrane-targeting signal, a phosphotyrosine-binding domain, a cleavable substrate for the protease, and a transcriptional effector protein; and
a second transcription unit encoding one or more viral replication proteins, wherein the second transcription unit is operably linked to a promoter activatable by the transcriptional effector protein.
   B2. The recombinant viral genome of embodiment B1, wherein the recombinant viral genome is that of a double-stranded DNA virus.
   B3. The recombinant viral genome of embodiment B2, wherein the double-stranded DNA virus is an adenovirus.
   B4. The recombinant viral genome of embodiment B3, wherein the one or more viral replication proteins comprise one or more E1 gene products.
   B5 The recombinant viral genome of any one of embodiments B1 to B4, wherein the transcriptional effector protein comprises a GAL4-VP16 transcription factor.
   B6. The recombinant viral genome of embodiment B5, wherein the promoter activatable by the transcriptional effector protein is a GAL4 UAS promoter.
   B7. A recombinant viral genome, comprising:
deletion of a viral gene; and
one or more transcription units encoding:
   a first fusion protein comprising a phosphotyrosine-binding domain and a protease, and
   a second fusion protein comprising, in N-terminal to C-terminal order or in C-terminal to N-terminal order, a membrane-targeting signal, a phosphotyrosine-binding domain, a cleavable substrate for the protease, and the viral protein encoded by the deleted gene.
      B8. The recombinant viral genome of embodiment B7, wherein the recombinant viral genome is that of an RNA virus.
      B9. The recombinant viral genome of embodiment B8, wherein the RNA virus is a negative-strand RNA virus.
      B10. The recombinant viral genome of embodiment B9, wherein the negative-strand RNA virus is a vesicular stomatitis virus (VSV).
      B11. The recombinant viral genome of embodiment B10, wherein the viral protein encoded by the deleted gene is VSV phosphoprotein (VSVP).
      B12. The recombinant viral genome of any one of embodiments B1 to B11, wherein the phosphotyrosine-binding domain of the first fusion protein and the second fusion protein is independently selected from a Src homology 2 (SH2) domain and a Shc domain.
      B13. The recombinant viral genome of any one of embodiments B1 to B12, wherein the protease is a viral protease.
      B14. The recombinant viral genome of embodiment B13, wherein the viral protease cleavage site is a hepatitis C virus (HCV) nonstructural protein 3 (NS3) protease.
      B15. The recombinant viral genome of any one of embodiments B1 to B14, wherein the membrane-targeting signal comprises a transmembrane domain.
      B16. The recombinant viral genome of embodiment B15, wherein the membrane-targeting signal comprises a membrane-tethering domain.
      B17. The recombinant viral genome of embodiment B16, wherein the membrane-tethering domain comprises a post-translational modification.
      B18. The recombinant viral genome of embodiment B17, wherein the post-translational modification comprises palmitoylation, myristoylation, prenylation, a glycosylphosphatidylinositol (GPI) anchor, or any combination thereof.
      B19. The recombinant viral genome of any one of embodiments B1 to B18, wherein the first fusion protein, the second fusion protein, or both, comprise a protein domain that emits a detectable signal.
      B20. The recombinant viral genome of embodiment B19, wherein the detectable signal is a fluorescent signal.
      B21. The recombinant viral genome of embodiment B19, wherein the detectable signal is a bioluminescent signal.
      B22. A cell comprising the recombinant viral genome of any one of embodiments B1 to B21.
      B23. The cell of embodiment B22, wherein the cell is a mammalian cell.
      B24. The cell of embodiment B22 or embodiment B23, wherein the cell is that of a viral packaging cell line.
      B25. Virions produced by the cell of embodiment B24.
      B26. Virions comprising the recombinant viral genome of any one of embodiments B1 to B21.
      B27. A pharmaceutical composition comprising the virions of embodiment B26.
      B28. A method comprising:
administering the virions of embodiment B26 to an individual having a disorder characterized by hyperactive ErbB signaling, wherein the virions are administered in an amount effective to induce death of cells exhibiting hyperactive ErbB signaling in the individual.
   B29. The method according to embodiment B28, wherein the disorder characterized by hyperactive ErbB signaling is a disorder characterized by hyperactive epidermal growth factor receptor (EGFR) signaling.
   B30. The method according to embodiment B28, wherein the disorder characterized by hyperactive ErbB signaling is a disorder characterized by hyperactive human EGF receptor 2 (HER2) signaling.
   B31. The method according to any one of embodiments B28 to B30, wherein the disorder characterized by hyperactive ErbB signaling is a cell proliferative disorder.
   B32. The method according to embodiment B31, wherein the cell proliferative disorder is cancer.
   B33. The method according to embodiment B32, wherein the cancer is selected from breast cancer, ovarian cancer, and non-small cell lung cancer.
   B34. A method of producing a conditionally replicating or nonreplicating virus of the order mononegalovirus, the method comprising:
(a) infecting a first mammalian cell population with vaccinia virus expressing bacteriophage T7 polymerase, wherein the first mammalian cell population comprises:
   (i) one or more plasmids expressing nucleocapsid, phosphoprotein, and the large (polymerase) protein for the species of the virus of the order mononegalovirus under the transcriptional control of T7 promoters, and
   (ii) a plasmid comprising a genome for the virus of the order mononegalovirus lacking phosphoprotein or large protein, or encoding phosphoprotein or large protein in a conditionally functional form, under the transcriptional control of a T7 promoter; and
(b) culturing a second mammalian cell population with the infected first mammalian cell population and/or a culture medium thereof, wherein the second mammalian cell population stably expresses nucleocapsid, phosphoprotein, and large (polymerase) protein, under conditions in which the second mammalian cell population produces the virus of the order mononegalovirus.
   B35. The method according to embodiment B34, wherein the virus of the order mononegalovirus is VSV.
   B36. The method according to embodiment B34 or embodiment B35, wherein the first mammalian cell population is a population of HEK293 cells or derivatives thereof.
   B37. The method according to embodiment B34 or embodiment B35, wherein the first mammalian cell population is a population of HEK293T cells.
   B38. The method according to any one of embodiments B34 to B37, wherein the second mammalian cell population is a population of BHK21 cells.
   B39. The method according to any one of embodiments B34 to B37, wherein the second mammalian cell population is a population of Vero cells.
   B40. The method according to embodiment B38 or embodiment B39, wherein the first mammalian cell population is a population of HEK293T cells, the second mammalian cell population is a population of BHK21 cells, and the virus of the order mononegalovirus is VSV.

The following examples are offered by way of illustration and not by way of limitation.

### EXPERIMENTAL

Described in the examples herein is the engineering of viruses that replicate selectively in response to hyperactive ErbB signaling. A synthetic two-protein system named ErbB-RASER (rewiring aberrant signaling to effector release) was previously developed to discern the difference between normal signaling and constitutive ErbB signaling (13). ErbB-RASER consists of a sequence-specific protease and a plasma membrane-localized substrate fused to an effector via a cognate substrate site. The two proteins contain phosphotyrosine-binding motifs for co-recruitment to active ErbB proteins, where proximity-enhanced substrate cleavage then releases the effector. As co-recruitment and cleavage continue as long as ErbB is phosphorylated, released effector levels reflect integration of ErbB signal over time. ErbB-RASER with an apoptosis-inducing effector previously outperformed standards of care in efficacy and specificity on HER2+ breast cancer cells (BT474) and EGFR+ lung cancer cells (H1975) in culture, indicating that ErbB-RASER output is highly selective for oncogenic ErbB signaling as opposed to normal signaling.

Assessed in the examples herein was whether it would be possible to construct a virus with ErbB-positive cancer-selective replication by incorporating ErbB-RASER into the viral life cycle (FIG. 1). To achieve RASER control of virus replication, a number of steps needed to be taken. First, the RASER components needed to be expressed from a location in the virus genome without perturbing the ability of the virus to proceed through its life cycle and to package the genome. Second, it was required to identify an early viral regulation step for control by ErbB-RASER so that the virus life cycle cannot proceed to a point of causing cytotoxicity in cells with normal ErbB signaling. Third, it was required to identify or construct an effector protein that is inactive when not released from the ErbB-RASER substrate fusion, but can promote viral progression through the life cycle after release. Fourth, the substrate-effector fusion required optimization to obtain cleavage selectively in ErbB-hyperactive cells and at the desired substrate site. Five, the overall ErbB-RASER system must be adjusted so that virus replication can complete in cancer cells prior to the virus genome becoming degraded, while minimizing breakthrough replication in non-cancer cells. It was not clear a priori that these multiple requirements could be simultaneously met for any virus.

To elucidate general principles for engineering viruses activated by ErbB signaling, the engineering of ErbB-RASER regulation into two viruses with distinct structures and life cycles was attempted: adenovirus, a protein-encapsulated double-stranded DNA virus, and vesicular stomatitis virus (VSV), a membrane-enveloped negative-strand RNA virus. The adenovirus life cycle begins with its DNA genome entering the cell nucleus, where a set of early regulatory genes are transcribed into messenger RNAs by host RNA polymerases. The regulatory proteins produced then induce later expression of genes encoding DNA polymerase and structural proteins. The virus DNA is then replicated and packaged into progeny virions inside the cytoplasm, which are then released as cells die, primarily through necrosis. The VSV life cycle begins with the negative-strand RNA genome and a small number of replicase complexes entering the cytoplasm. The replicase then transcribes messenger RNAs for all five viral genes. The resulting newly synthesized replicase complexes and structural proteins then allow formation of a full-length positive RNA genome strand, followed by amplification of full-length progeny negative genomes, recruitment of genomes to the membrane, and budding of new virions from the membrane. Cells die primarily through necrosis as well.

### Example 1—Engineering of an Adenovirus Regulated by ErbB Hyperactivity

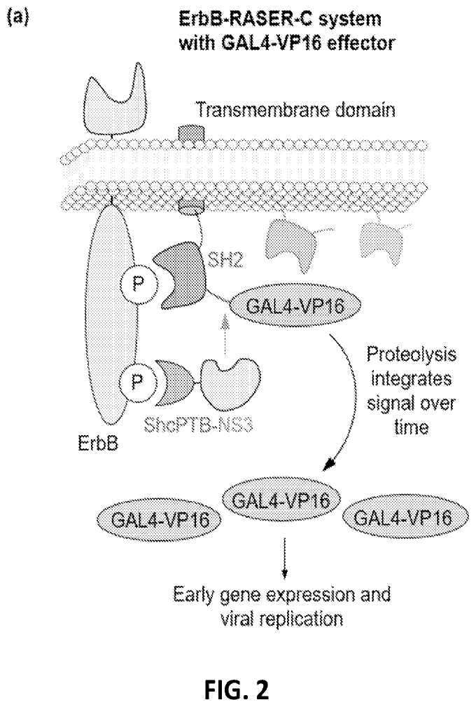

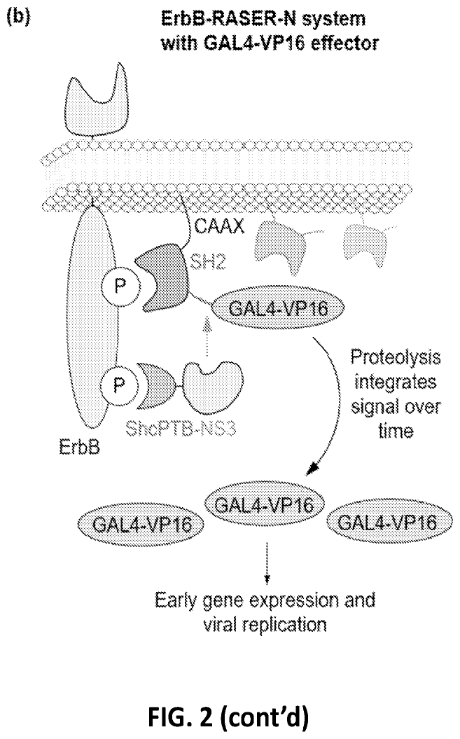

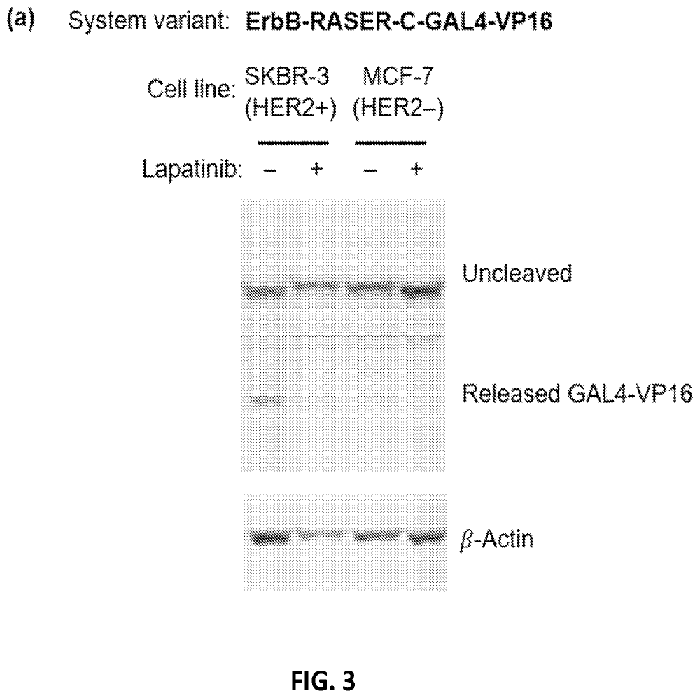

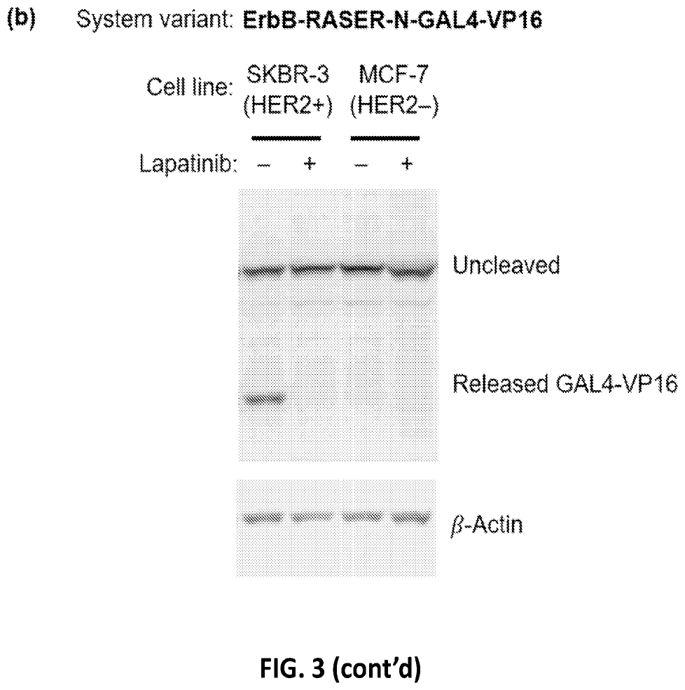

To engineer an adenovirus regulated by ErbB hyperactivity, an ErbB-RASER substrate fusion protein was first created that releases the GAL4-VP16 transcription factor upon ErbB-dependent cleavage by the protease fusion (FIG. 2). Two versions of the substrate fusion protein were tested, one with an N-terminal transmembrane domain for membrane localization, with the GAL4-VP16 effector fused to the C-terminus, and another with the GAL4-VP16 effector fused to the N-terminus, with a C-terminal CAAX sequence for membrane localization (FIG. 2). It was found that both systems released GAL4-VP16 when expressed in a HER2-positive breast cancer cell line but not in a HER2-negative breast cancer cell line (FIG. 3). GAL4-VP16 release was dependent on HER2 kinase activity, as it was inhibited by the ErbB inhibitor lapatinib. The adenovirus serotype 5 (Ad5) genome was then modified as follows (FIG. 4): (1) the ErbB-RASER protease and substrate fusions were expressed from a transcription unit driven by the constitutive CMV promoter placed upstream of the E1A/B gene, (2) the E1 promoter was replaced with a GAL4 UAS promoter.

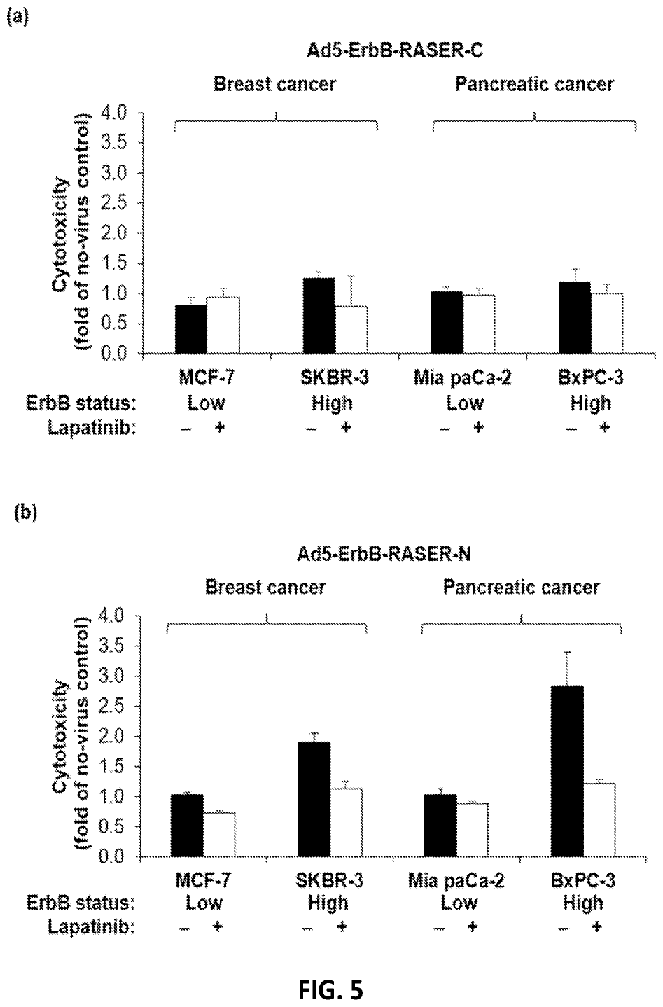

Ad5-ErbB-RASER-C and Ad5-ErbB-RASER-N were first tested for cytotoxicity in cancer cells that were either ErbB-positive, i.e., hyperactive for EGFR or HER2 signaling, or tissue-matched cancer cells that were ErbB-negative. To determine whether any effects were due to ErbB activity, the assessment was also made in the absence or presence of the ErbB activity inhibitor lapatinib. Ad5-ErbB-RASER-N showed greater toxicity on ErbB-positive cells than Ad5-RASER-C (FIG. 5). While the reason for this difference is unclear, it is a functionally important difference that could not be predicted and was derived from experimentation. It is possible that expression of the RASER-C substrate fusion is relatively impaired, or is not well colocalized with ErbB at the plasma membrane.

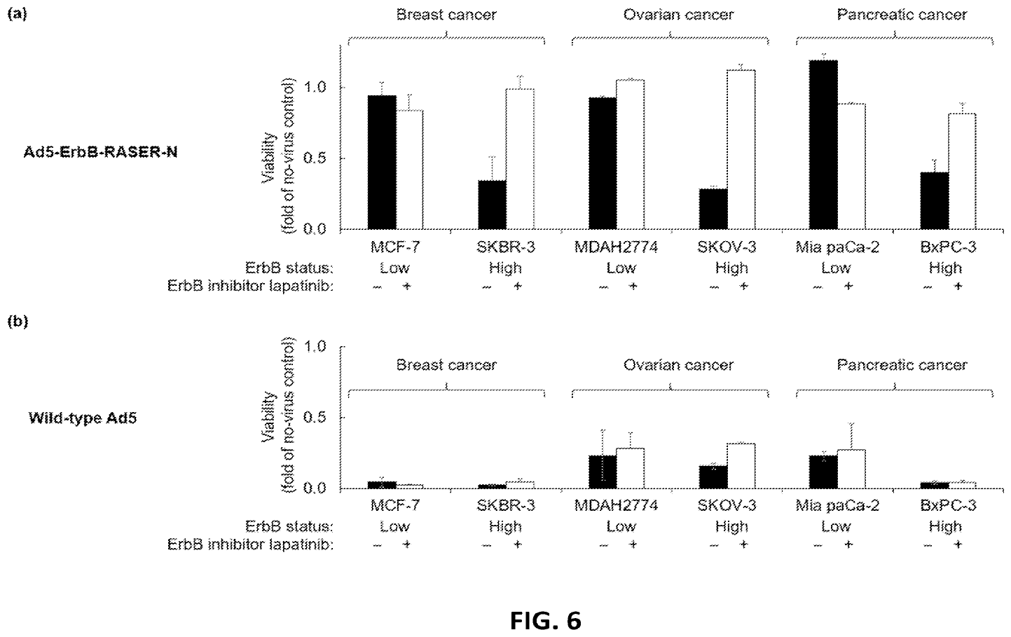

To further test Ad5-ErbB-RASER-N performance and specificity, viability assays were performed to determine the percentage of live cells remaining following Ad5-ErbB-RASER-N infection at different times. At day 6 post-infection, a specific ability of Ad5-ErbB-RASER-N to kill ErbB-positive cell lines was observed (FIG. 6a), and again could be suppressed by lapatinib, as desired. Also as desired, Ad5-ErbB-RASER-N was completely inactive on ErbB-negative cells. However, even at the prolonged 6-day incubation times, Ad5-ErbB-RASER-N was unable to completely eliminate ErbB-positive cells. Wild-type Ad5 was more effective in cellular elimination but did so regardless of ErbB-positivity (FIG. 6b). In ErbB-negative cells, wild-type Ad5 was capable of causing more cytotoxicity than Ad5-ErbB-RASER-N (comparing FIGS. 6a and 6b), demonstrating that the RASER switch was limiting virus-mediated toxicity in ErbB-negative cells, and confirming these lines can be infected with Ad5.

Interestingly, Ad5-RASER-N was suppressed to baseline levels in ErbB-positive cancer lines (where baseline is 1-fold of no-virus control) by lapatinib, demonstrating cytotoxicity required ErbB activity, as desired (FIG. 6b). These results thus indicate that Ad5-ErbB-RASER-N demonstrates the desired specificity, but is not as effective as wild-type adenovirus in killing cancer cells.

### Example 2—Engineering of RNA Viruses Regulated by ErbB Hyperactivity

Attempted next was the engineering of a different class of oncotropic viruses based on measles virus (MV) or vesicular stomatitis virus (VSV), RNA viruses of the order mononegaloviridae that replicate entirely in the cytosol using their own polymerase protein (FIG. 7). These viruses are challenging to regulate as the transcription of individual genes is not controlled by transcription factors. Rather the viral RNA-dependent RNA polymerase initiates and terminates transcription of each gene, using the antisense genomic strand as a template.

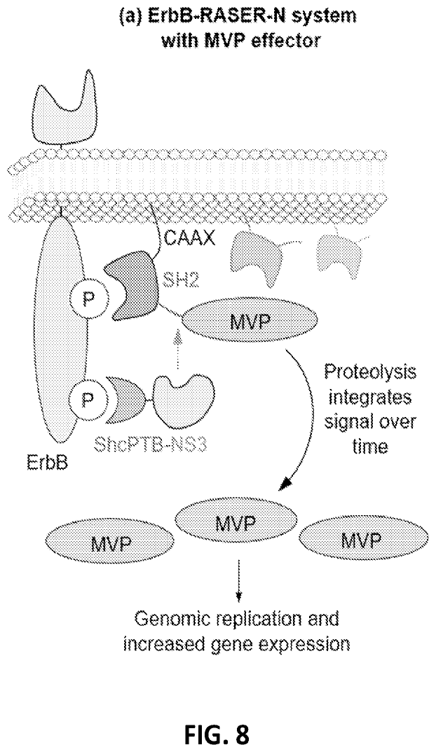

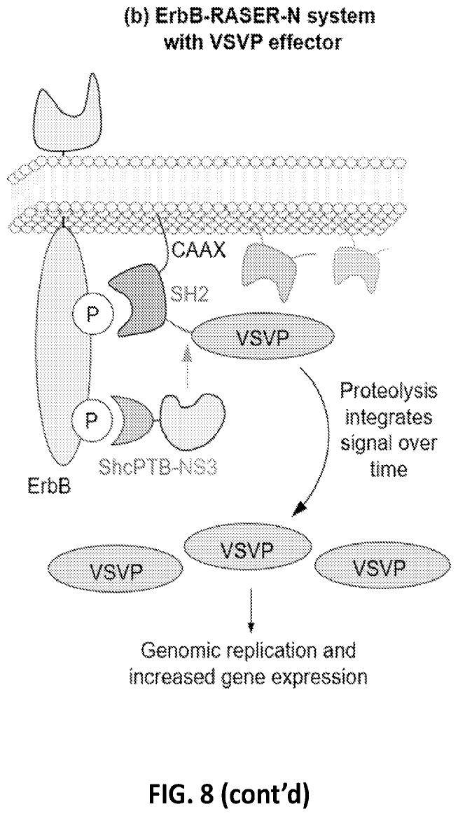

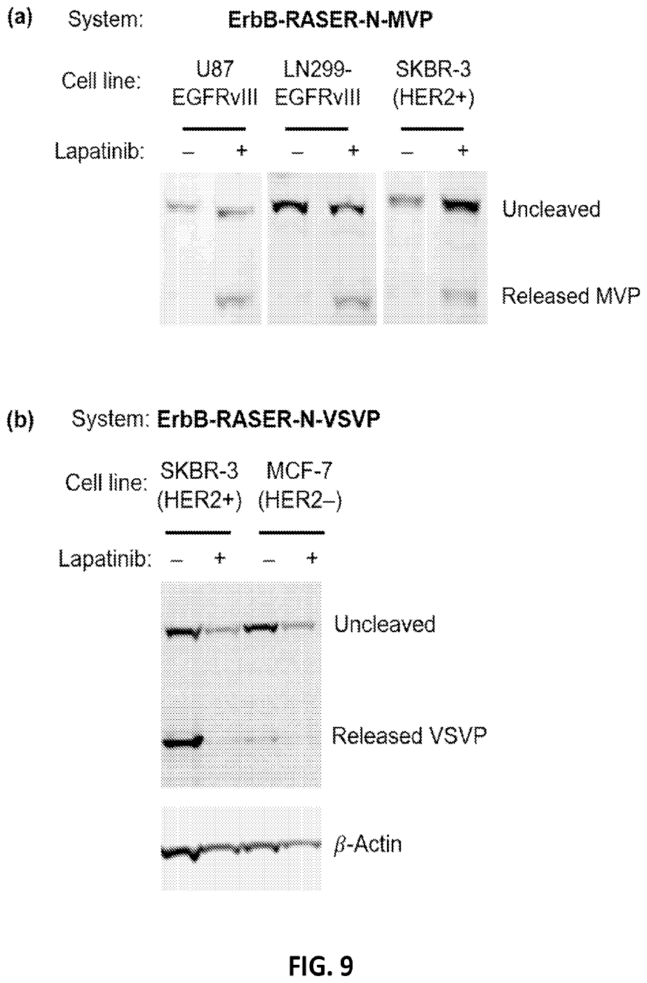

To engineer a MV or VSV regulated by ErbB hyperactivity, MVP-substrate-SH2-CAAX, an ErbB-RASER substrate fusion that releases the MV phosphoprotein (MVP) from the N-terminus upon ErbB-dependent cleavage by the ShcPTB-NS3 protease fusion protein, was created (FIG. 8a). Likewise, VSVP-substrate-SH2-CAAX, an ErbB-RASER substrate fusion that releases the VSV phosphoprotein (VSVP) from the N-terminus upon ErbB-dependent cleavage by the ShcPTB-NS3 protease fusion protein, was created (FIG. 8b). It was found that these systems released MV or VSVP when expressed in HER2-positive cancer cells but not in HER2-negative cancer cells (FIG. 9). MV or VSVP release was dependent on HER2 kinase activity, as it was inhibited by the ErbB kinase inhibitor lapatinib.

Next, working with pT7-MV-GFP, a DNA plasmid in which the entire reverse-transcribed genome of a recombinant MV with an additional green fluorescent protein transcription unit is placed under the transcriptional control of the bacteriophase T7 polymerase, plasmid pT7-MV-ErbB-RASER-N was made as follows (FIG. 10a): (1) the GFP open reading frame was fused to a P2A translational skipping sequence followed by the open reading frame for the ShcPTB-NS3 protease fusion protein, and (2) the MVP coding sequences were replaced with MVP-substrate-SH2-CAAX coding sequences. pT7-VSV-YFP, a recombinant VSV with an additional yellow fluorescent protein transcription unit, was also modified to make pT7-VSV-ErbB-RASER-N as follows (FIG. 10b): the VSVP coding sequence was replaced with one encoding both ErbB-RASER-N components (ShcPTB-NS3 and VSVP-substrate-SH2-CAAX) separated by the P2A ribosome-skipping sequence.

Established methods to recover (or rescue) live virus from MV or VSV plasmids involve transfecting the genome-containing plasmid into mammalian cells that also express the nucleocapsid, phosphoprotein, and large (polymerase) viral proteins and the polymerase of the bacteriophage T7. The T7 polymerase performs transcription of a positive strand (sense) RNA genome from the genomic plasmid from the T7 promoter sequence. The resulting positive strand of RNA serves as a template for the generation by the replicase complex (consisting of nucleocapsid, phosphoprotein, and large protein) of complete genomic negative strands which are then transcribed and packaged into virions. Cell culture supernatants from these transfected cells, or the cells themselves, are then put in contact with a second virus-permissive cell line, such as Vero or BHK21 cells, to further amplify the virus.

To amplify the MV-ErbB-RASER-N or VSV-ErbB-RASER-N viruses, MVP or VSVP protein must be present in the amplifying cell type. If the cell type exhibits hyperactive ErbB signaling, MVP or VSVP protein will be released by ErbB signal-dependent cleavage from the MVP-substrate-SH2-CAAX or VSV-substrate-SH2-CAAX fusion protein expressed by the virus. However, these proteins are only expressed after viral entry, and time will be required for released MVP or VSVP to accumulate to allow replication of the virus to continue. It is thus advantageous to engineer the cell line used for amplification to express MVP or VSVP protein constitutively, so that the cell contains abundant MVP or VSVP protein even before viruses enter.

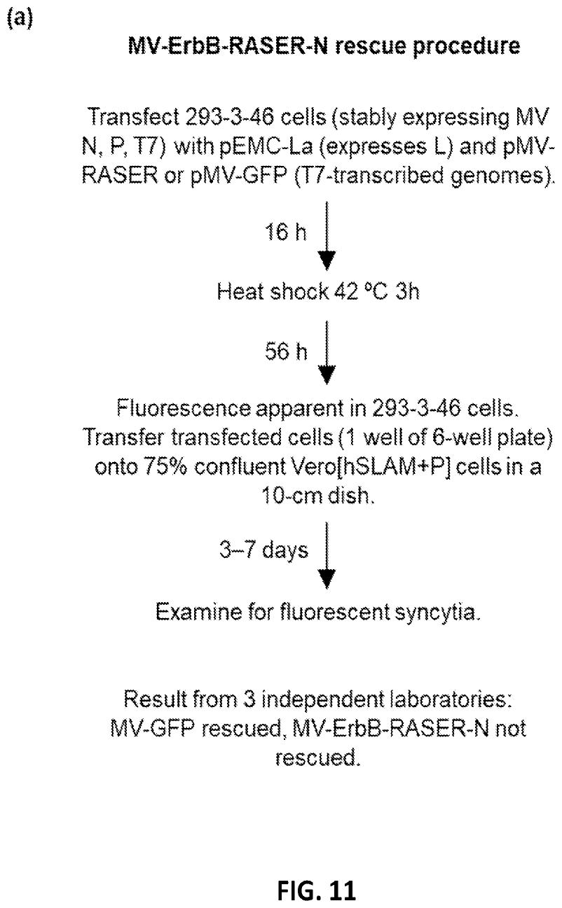

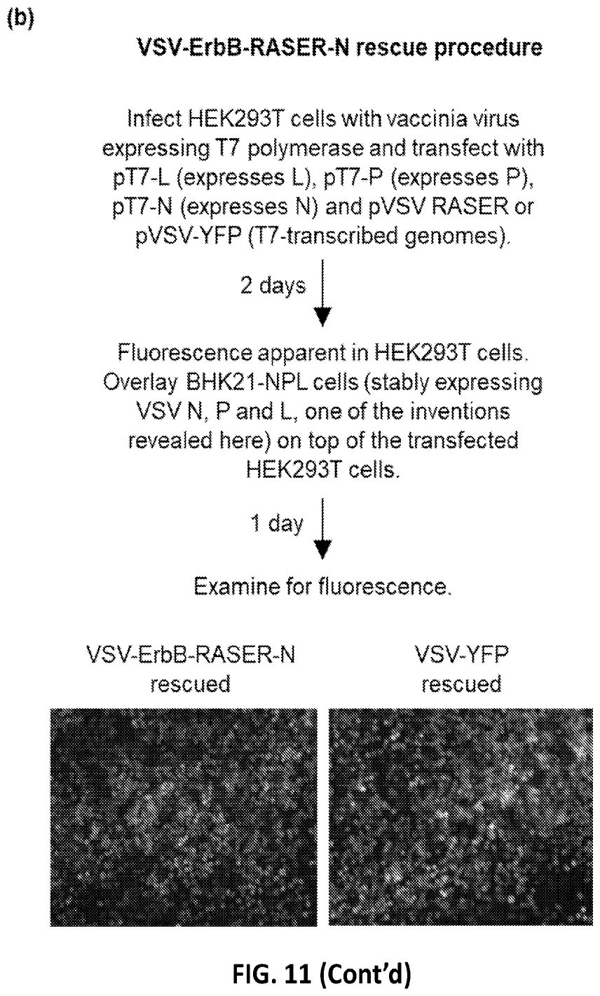

To recover MV-ErbB-RASER-N virus, Vero cells stably expressing SLAM (a receptor for MV) and MVP were generated. Then, recovery of MV-ErbB-RASER-N virus was attempted by adapting an established protocol used to package a MV with a drug-controllable phosphoprotein (14). Specifically, pT7-MV-ErbB-RASER-N and an expression plasmid encoding MV large protein were transfected into the 293-3-46 cell line that expresses T7, MV nucleocapsid, and MV phosphoprotein, and the Vero-SLAM-MVP line was used in place of standard Vero cells during the following amplification step. However, this approach did not result in successful recovery of live virus (FIG. 11a).

Recovery of live virus from the pT7-VSV-ErbB-RASER-N plasmid was then attempted. One published protocol instructed transfecting into HEK293 cells the pT7-VSV-ErbB-RASER-N plasmid and plasmids that express T7 polymerase, VSV nucleocapsid (VSVN), VSVP, VSV large (VSVL), and VSV glycoprotein (VSVG) from a constitutive CAG promoter, then amplifying in BHK21 cells (15). Here, BHK21 cells stably expressing VSVP (BHK21-VSVP) were generated and used in place of standard BHK21 cells during the amplification step. Another protocol instructed infecting BHK21 cells with vaccinia virus expressing T7 polymerase, then transfecting them with pT7-VSV-ErbB-RASER-N and plasmids that express VSVN, VSVP, and VSVL cells from a combined CMV and T7 promoter (16). Here, BHK21-VSVP was used in place of BHK21 cells in both of these procedures, but they failed to produce live virus from the pT7-VSV-ErbB-RASER-N plasmid.

Described herein are new materials and a new method for recovering recombinant VSV with conditional VSVP function from genomic plasmids. First, BHK21 cells stably expressing VSVN, VSVP, and VSVL proteins (BHK21-NPL cells) were generated. Next, HEK293T cells were infected with vaccinia virus expressing T7 polymerase, followed by transfection with the pT7-VSV-ErbB-RASER-N genomic plasmid and plasmids that express VSVN, VSVP, and VSVL from a T7 promoter. Two days later, the cells were overlaid with the BHK21-NPL cells. This procedure was successful in generating VSV-ErbB-RASER-N viruses (FIG. 11b).

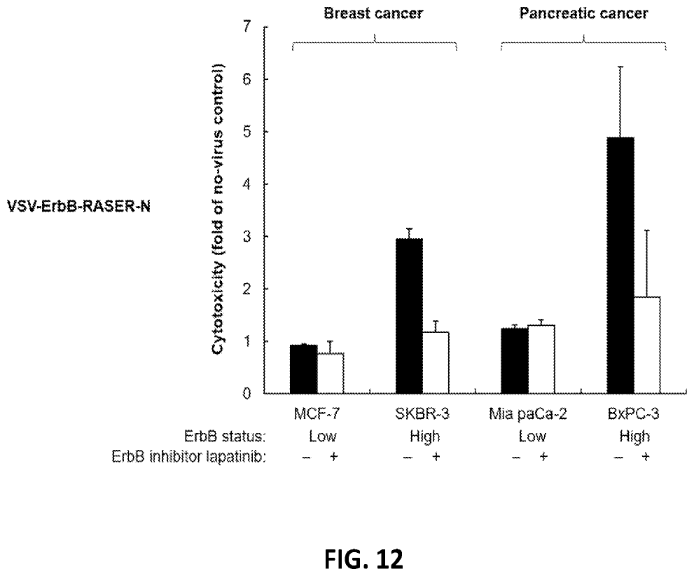

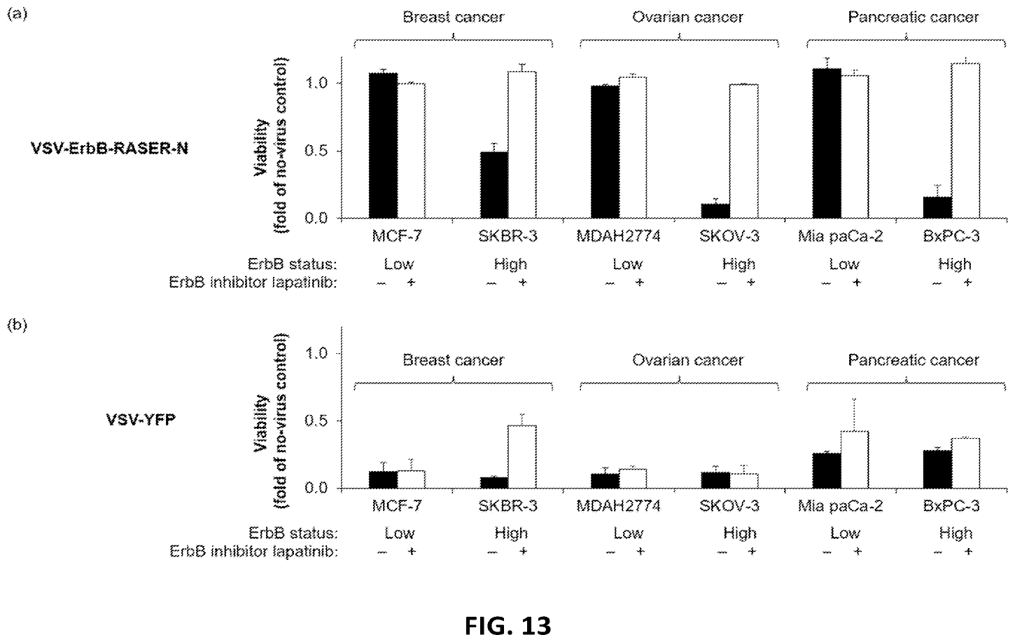

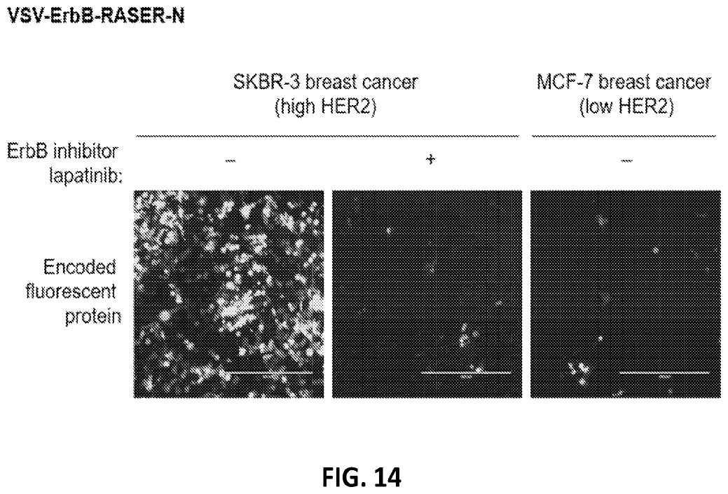

The resulting VSV-ErbB-RASER-N virus demonstrated specific cytotoxicity to ErbB-positive cells after 2 days (FIG. 12) and a clear reduction of cell viability after 4 days (FIG. 13). Notably, VSV-ErbB-RASER-N was as active as wild-type VSV on untreated ErbB-positive cells, but not on ErbB-negative cells or cells treated with lapatinib (FIG. 13). Efficacy and specificity of VSV-ErbB-RASER-N held across cancer cells from different tissue types, including breast cancer cells, ovarian cancer cells and pancreatic cancer cells, indicating broad applicability of VSV-ErbB-RASER-N viruses to multiple types of cancer (FIG. 13). Imaging of the YFP reporter in VSV-ErbB-RASER-N indicated nearly complete specificity in viral replication in ErbB-positive cells that was dependent on ErbB activity (FIG. 14).

### SUMMARY

Adenoviruses and VSV are both currently in clinical trials for cancer, by either local or systemic delivery. They are also already approved as standard vaccine vectors for viral diseases. However, adenoviruses and VSV viruses in trials for cancer therapy lack restriction to cancer cells with hyperactive signals. The Ad-ErbB-RASER-N and VSV-ErbB-RASER-N viruses described here thus are suitable for use within existing treatment approaches for cancer without further modification.

The ability of VSV-ErbB-RASER-N to kill the ErbB-positive pancreatic cancer line BxPC-3 is of particular interest as this line is resistant to killing by previous VSV variants developed as cancer treatments. Specifically, VSV-ΔM51, a recombinant VSV with a deletion of amino acid 51 in the matrix protein, is defective for interferon response evasion, and therefore replicates more poorly in normal cells with intact interferon signaling pathways than in cells with defective interferon signaling. While defective interferon signaling is characteristic of many cancer cell lines (17), some lines such as BxPC-3 have intact interferon signaling and are resistant to killing by VSV-ΔM51 (18). In addition, VSV-ΔM51 demonstrates attenuation relative to the wild-type VSV and poor activity in immunocompetent pancreatic cancer mouse models (5). By using a non-mutant matrix protein, VSV-ErbB-RASER-N is apparently able to replicate in and kill BxPC-3 cells, whereas VSV-ΔM51 was not. This indicates the greater lethality of VSV-ErbB-RASER than VSV-ΔM51 in cells with intact interferon signaling.

In addition, the ability of VSV-ErbB-RASER-N to spare ErbB-negative breast cancer cells is unprecedented as both VSV-ΔM51 and wild-type VSV were found to indiscriminately kill breast cancer and breast normal epithelial cells (4). That is, in breast cancer, no VSV variant had previously been engineered to be cancer-specific. In addition, while VSV-ΔM51 was found to be less lethal than wild-type VSV in multiple breast cancer lines (4), it was observed here that VSV-ErbB-RASER-N was as lethal as wild-type VSV in ErbB+ cells. Thus, as designed, VSV-ErbB-RASER-N specifically kills ErbB+ breast cancer cells while maintaining similar cytotoxicity as wild-type VSV in those cells.

To summarize, described herein is an anti-cancer therapeutic in the form of viruses which conditionally replicate and cause cell death in a ErbB hyperactivity-dependent manner. This work represents the first engineering of viral replication to be dependent on a hyperactive biochemical signal inside cancer cells, without depending on the presence of endogenous tumor-selective transcription factors. This work represents a new paradigm for treating cancer, in which synthetic proteins and engineered viruses selectively kill cancer cells based on well characterized biochemical drivers of tumorigenesis.

As will be appreciated by those of ordinary skill in the art of molecular biology, the methods of constructing ErbB-gated adenoviruses described herein encompass many variations. For example, with the benefit of the present disclosure, alternative ways to co-express the two RASER components are possible. These include, e.g., replacing P2A with other translation-interrupting sequences, replacing P2A with an internal ribosome entry site (IRES), replacing P2A with a segment containing a polyadenylation signal and a promoter, or flanking the upstream open reading frame with splice-donor and splice acceptor sites, each with or without reversing the order of the RASER components. In addition, with the benefit of the present disclosure, it is expected that the CMV promoter can be replaced with another strong constitutive promoter, and that GAL4-VP16 can be replaced with another transcription factor and the 5xUAS replaced with a promoter sequence activated by that transcription factor. Moreover, beneficial alterations in adenovirus genomes outside of E1 regulation described in the prior art, such as the addition of cytokine genes, can be combined with the methods for RASER regulation of E1 expression described herein. Finally, the methods described herein apply directly to any other adenovirus species with an E1 gene.

Likewise, the principles revealed here for making ErbB-gated VSV via phosphoprotein release lead directly to variations in virus structure that achieve the same principles but in different ways. For example, the RASER components were expressed as one transcription unit with a P2A site to separate the two components in the example described, but these two components could be expressed from two different transcription units, which can be in different orders, and can be located in any intergenic regions in the VSV genome, as all genes are constitutively transcribed, although those further upstream are transcribed more efficiently. The two RASER components can also be co-expressed in tandem, by replacing the P2A sequence with other translation-interrupting sequences or an IRES, either with or without reversing the components.

### REFERENCES

1. Liu B L, Robinson M, Han Z Q, Branston R H, English C, Reay P, McGrath Y, Thomas S K, Thornton M, Bullock P et al. (2003) ICP34.5 deleted herpes simplex virus with enhanced oncolytic, immune stimulating, and anti-tumour properties. Gene Ther 10:292-303.
2. Harrington K J, Kong A, Mach N, Chesney J A, Fernandez B C, Rischin D, Cohen E E W, Radcliffe H S, Gumuscu B, Cheng J et al. (2020) Talimogene Laherparepvec and Pembrolizumab in Recurrent or Metastatic Squamous Cell Carcinoma of the Head and Neck (MASTERKEY-232): A Multicenter, Phase 1b Study. Clin Cancer Res 26:5153-5161.
3. Stojdl D F, Lichty B D, tenOever B R, Paterson J M, Power A T, Knowles S, Marius R, Reynard J, Poliquin L, Atkins H et al. (2003) VSV strains with defects in their ability to shutdown innate immunity are potent systemic anti-cancer agents. Cancer Cell 4:263-275.
4. Ahmed M, Puckett S, Lyles D S (2010) Susceptibility of breast cancer cells to an oncolytic matrix (M) protein mutant of vesicular stomatitis virus. Cancer Gene Ther 17:883-892.
5. Hastie E, Besmer D M, Shah N R, Murphy A M, Moerdyk-Schauwecker M, Molestina C, Roy L D, Curry J M, Mukherjee P, Grdzelishvili V Z (2013) Oncolytic vesicular stomatitis virus in an immunocompetent model of MUC1-positive or MUC1-null pancreatic ductal adenocarcinoma. J Virol 87:10283-10294.
6. Jenks N, Myers R, Greiner S M, Thompson J, Mader E K, Greenslade A, Griesmann G E, Federspiel M J, Rakela J, Borad M J et al. (2010) Safety studies on intrahepatic or intratumoral injection of oncolytic vesicular stomatitis virus expressing interferon-beta in rodents and nonhuman primates. Hum Gene Ther 21:451-462.
7. Zhang L, Steele M B, Jenks N, Grell J, Suksanpaisan L, Naik S, Federspiel M J, Lacy M Q, Russell S J, Peng K W (2016) Safety Studies in Tumor and Non-Tumor-Bearing Mice in Support of Clinical Trials Using Oncolytic VSV-IFNβ-NIS. Hum Gene Ther Clin Dev 27:111-122.
8. Bazan-Peregrino M, Carlisle R C, Hernandez-Alcoceba R, Iggo R, Homicsko K, Fisher K D, Hallden G, Mautner V, Shen Y, Seymour L W (2008) Comparison of molecular strategies for breast cancer virotherapy using oncolytic adenovirus. Hum Gene Ther 19:873-886.
9. Kirn D (2001) Clinical research results with dl1520 (Onyx-015), a replication-selective adenovirus for the treatment of cancer: what have we learned. Gene Ther 8:89-98.
10. Torti D, Trusolino L (2011) Oncogene addiction as a foundational rationale for targeted anti-cancer therapy: promises and perils. EMBO Mol Med 3:623-636.
11. Mishra R, Hanker A B, Garrett J T (2017) Genomic alterations of ERBB receptors in cancer: clinical implications. Oncotarget 8:114371-114392.
12. Whilding L M, Maher J (2015) ErbB-targeted CAR T-cell immunotherapy of cancer. Immunotherapy 7:229-241.
13. Chung H K, Zou X, Bajar B T, Brand V R, Huo Y, Alcudia J F, Ferrell J E, Lin M Z (2019) A compact synthetic pathway rewires cancer signaling to therapeutic effector release. Science 364
14. Chung H K, Jacobs C L, Huo Y, Yang J, Krumm S A, Plemper R K, Tsien R Y, Lin M Z (2015) Tunable and reversible drug control of protein production via a self-excising degron. Nat Chem Biol 11:713-720.
15. Takahashi K, Yokobayashi Y (2019) Reversible Gene Regulation in Mammalian Cells Using Riboswitch-Engineered Vesicular Stomatitis Virus Vector. ACS Synth Biol 8:1976-1982.
16. Ammayappan A, Nace R, Peng K W, Russell S J (2013) Neuroattenuation of vesicular stomatitis virus through picornaviral internal ribosome entry sites. J Virol 87:3217-3228.
17. Moerdyk-Schauwecker M, Shah N R, Murphy A M, Hastie E, Mukherjee P, Grdzelishvili V Z (2013) Resistance of pancreatic cancer cells to oncolytic vesicular stomatitis virus: role of type I interferon signaling. Virology 436:221-234.
18. Murphy A M, Besmer D M, Moerdyk-Schauwecker M, Moestl N, Ornelles D A, Mukherjee P, Grdzelishvili V Z (2012) Vesicular stomatitis virus as an oncolytic agent against pancreatic ductal adenocarcinoma. J Virol 86:3073-3087.

Accordingly, the preceding merely illustrates the principles of the present disclosure. It will be appreciated that those skilled in the art will be able to devise various arrangements which, although not explicitly described or shown herein, embody the principles of the present disclosure and are included within its spirit and scope. Furthermore, all examples and conditional language recited herein are principally intended to aid the reader in understanding the principles of the present disclosure and the concepts contributed by the inventors to furthering the art, and are to be construed as being without limitation to such specifically recited examples and conditions. Moreover, all statements herein reciting principles, aspects, and embodiments of the present disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein.

What is claimed is:

1. A recombinant viral genome comprising:
(a) a first transcription unit comprising:
(i) a first sequence encoding a first fusion protein comprising a protease and a first binding domain that binds to a first target, and
(ii) a second sequence encoding a second fusion protein comprising, in N-terminal to C-terminal order or in C-terminal to N-terminal order, a membrane-targeting signal, a second binding domain that binds to a second target, a substrate cleavable by the protease, and a transcriptional effector protein; and
(b) a second transcription unit comprising a third sequence encoding one or more viral replication proteins, wherein the second transcription unit is operably linked to a promoter activatable by the transcriptional effector protein.

2. The recombinant viral genome of claim 1, wherein the recombinant viral genome comprises a modified genome of a virus.

3. The recombinant viral genome of claim 2, wherein the virus is an oncolytic virus.

4. The recombinant viral genome of any one of claims 1 to 3, wherein the promoter is not activatable by the transcriptional effector protein until the transcriptional effector protein is cleaved from the second fusion protein.

5. The recombinant viral genome of any one of claims 1 to 3, wherein the promoter is activatable to a lesser extent by the second fusion protein compared to the transcriptional effector protein when cleaved from the second fusion protein.

6. The recombinant viral genome of any one of claims 1 to 5, wherein the transcriptional effector protein comprises a transcription factor.

7. The recombinant viral genome of any one of claims 2 to 6, wherein the one or more viral replication proteins encoded by the second transcription unit is required for replication of the virus in cells or efficient replication of the virus in cells.

8. The recombinant viral genome of any one of claims 2 to 7, wherein replication of the virus in cells is dependent on a signal specific to cancer cells or a signal in cancer cells that is higher than the signal in non-cancer cells.

9. The recombinant viral genome of any one of claims 2 to 8, wherein replication of the virus in cells is not dependent on the presence of an endogenous transcription factor.

10. The recombinant viral genome of claim 9, wherein the endogenous transcription factor is an endogenous cancer-selective transcription factor.

11. The recombinant viral genome of any one of claims 1 to 10, wherein the recombinant viral genome further comprises a third transcription unit encoding an immunostimulatory molecule, optionally wherein the immunostimulatory molecule is granulocyte/macrophage colony stimulating factor (GM-CSF).

12. The recombinant viral genome of any one of claims 2 to 11, wherein the virus is a double-stranded DNA virus.

13. The recombinant viral genome of claim 12, wherein the double-stranded DNA virus is an adenovirus.

14. The recombinant viral genome of claim 13, wherein the one or more viral replication proteins comprise one or more E1 gene products.

15. The recombinant viral genome of any one of claims 1 to 14, wherein the transcriptional effector protein comprises a GAL4-VP16 transcription factor.

16. The recombinant viral genome of claim 15, wherein the promoter activatable by the transcriptional effector protein is a GAL4 UAS promoter.

17. A recombinant viral genome, comprising:
(a) a deficiency in at least one native viral gene; and
(b) one or more transcription units comprising:
(i) a sequence encoding a first fusion protein comprising a protease and a first binding domain that binds to a first target, and
(ii) a sequence encoding a second fusion protein comprising, in N-terminal to C-terminal order or in C-terminal to N-terminal order, a membrane-targeting signal, a second binding domain that binds to a second target, a substrate cleavable by the protease, and the viral protein encoded by the deleted gene.

18. The recombinant viral genome of claim 17, wherein the recombinant viral genome comprises a modified genome of a virus.

19. The recombinant viral genome of claim 18, wherein the virus is a wild-type virus.

20. The recombinant viral genome of claim 18, wherein the virus is an engineered virus.

21. The recombinant viral genome of claim 18, wherein the virus is an RNA virus.

22. The recombinant viral genome of claim 19, wherein the RNA virus is a negative-strand RNA virus.

23. The recombinant viral genome of claim 22, wherein the negative-strand RNA virus is a mononegavirus.

24. The recombinant viral genome of claim 23, wherein the mononegavirus is a rhabdovirus.

25. The recombinant viral genome of claim 24, wherein the rhabdovirus is a vesicular stomatitis virus (VSV).

26. The recombinant viral genome of claim 25, wherein the viral protein encoded by the deleted gene is VSV phosphoprotein (VSVP).

27. The recombinant viral genome of any one of claims 1 to 26, wherein the first and second targets are present on the same protein.

28. The recombinant viral genome of claim 27, wherein the first and second targets are different epitopes of the same protein.

29. The recombinant viral genome of any one of claims 1 to 26, wherein the first and second targets are present on different proteins.

30. The recombinant viral genome of claim 29, wherein the different proteins form a complex with each other.

31. The recombinant viral genome of claim 30, wherein the complex is present at a higher level in cancer cells than non-cancer cells.

32. The recombinant viral genome of any one of claims 1 to 28, wherein the first and second targets are present on one or more proteins that are specifically expressed by cancer cells.

33. The recombinant viral genome of any one of claims 1 to 28, wherein the first and second targets are present on one or more proteins that are expressed at a higher level in cancer cells compared to non-cancer cells.

34. The recombinant viral genome of any one of claim 1 to 28, 32, or 33, wherein the first binding domain is a phosphotyrosine binding domain.

35. The recombinant viral genome of any one of claim 1 to 28, 32, or 33, wherein the second binding domain is a phosphotyrosine binding domain.

36. The recombinant viral genome of any one of claim 1 to 28, 32, or 33, wherein the first binding domain is a phosphotyrosine binding domain and the second binding domain is a phosphotyrosine binding domain.

37. The recombinant viral genome of any one of claims 1 to 36, wherein the first and second targets are in proximity to each other such that the protease cleaves the cleavable substrate when the first and second binding domains are bound to their respective targets.

38. The recombinant viral genome of claim 27, wherein the same protein is a receptor or a transmembrane protein.

39. The recombinant viral genome of claim 27, wherein the same protein is hyperactive or signals constitutively.

40. The recombinant viral genome of claim 27, wherein the same protein is a mutant protein.

41. The recombinant viral genome of claim 27, wherein the same protein is human EGF receptor 2 (HER2).

42. The recombinant viral genome of claim 27, wherein the same protein is epidermal growth factor receptor (EGFR).

43. The recombinant viral genome of any one of claims 1 to 42, wherein the first binding domain of the first fusion protein and the second binding domain of the second fusion protein is independently selected from a Src homology 2 (SH2) domain and a Shc domain.

44. The recombinant viral genome of any one of claims 1 to 43, wherein the protease is a viral protease.

45. The recombinant viral genome of claim 44, wherein the viral protease is a hepatitis C virus (HCV) nonstructural protein 3 (NS3) protease.

46. The recombinant viral genome of any one of claims 1 to 45, wherein the membrane-targeting signal comprises a transmembrane domain.

47. The recombinant viral genome of claim 46, wherein the membrane-targeting signal comprises a membrane-tethering domain.

48. The recombinant viral genome of claim 47, wherein the membrane-tethering domain comprises a post-translational modification.

49. The recombinant viral genome of claim 48, wherein the post-translational modification comprises palmitoylation, myristoylation, prenylation, a glycosylphosphatidylinositol (GPI) anchor, or any combination thereof.

50. The recombinant viral genome of any one of claims 1 to 49, wherein the first fusion protein, the second fusion protein, or both, comprise a protein domain that emits a detectable signal.

51. A cell comprising the recombinant viral genome of any one of claims 1 to 50.

52. The cell of claim 51, wherein the cell is a mammalian cell.

53. The cell of claim 51 or claim 52, wherein the cell is that of a viral packaging cell line.

54. A virion produced by the cell of claim 53.

55. A virion comprising the recombinant viral genome of any one of claims 1 to 50.

56. A pharmaceutical composition comprising a plurality of virions of claim 55.

57. A method of treating a disease or condition in a subject in need thereof comprising administering the pharmaceutical composition of claim 56 to the subject.

58. The method of claim 57, wherein the pharmaceutical composition comprises the virions in an amount effective to induce death of cells exhibiting a signal specific to the disease or disorder in the subject.

59. The method of claim 57, wherein the pharmaceutical composition comprises the virions in an amount effective to induce death of cells exhibiting a signal that is higher than the signal in non-diseased cells.

60. The method of claim 57, wherein the disease or condition is a cancer.

61. The method of claim 57, wherein the disease or condition is characterized by hyperactive ErbB signaling.

62. The method according to claim 61, wherein the disease or condition characterized by hyperactive ErbB signaling is a disorder characterized by hyperactive epidermal growth factor receptor (EGFR) signaling.

63. The method according to claim 61, wherein the disease or condition characterized by hyperactive ErbB signaling is a disease or condition characterized by hyperactive human EGF receptor 2 (HER2) signaling.

64. The method according to claim 63, wherein the disease or condition characterized by hyperactive ErbB signaling is a cell proliferative disease or condition.

65. The method according to claim 64, wherein the cell proliferative disease or condition is a cancer.

66. The method according to claim 65, wherein the cancer is selected from breast cancer, ovarian cancer, and non-small cell lung cancer.

67. A method of producing a conditionally replicating or nonreplicating mononegavirus, the method comprising:
(a) infecting a first cell population with a virus expressing a DNA sequence-specific RNA polymerase, wherein the first cell population comprises:
(i) one or more plasmids containing one or more genes encoding a nucleocapsid, a phosphoprotein, and a large protein of a mononegavirus wherein the one or more genes are linked to a DNA sequence recognized by the RNA polymerase, and
(ii) a plasmid comprising a genome of the mononegavirus linked to the DNA sequence recognized by the DNA sequence-specific RNA polymerase; and
(b) culturing a second cell population with the first cell population and/or a culture medium thereof, wherein the second cell population expresses a nucleocapsid, a phosphoprotein, and a large protein, under one or more conditions in which the second cell population produces the mononegavirus.

68. The method according to claim 67, wherein the DNA sequence-specific RNA polymerase is a bacteriophage T7 RNA polymerase.

69. The method according to claim 67, wherein the mononegavirus is a rhabdovirus.

70. The method according to claim 69, wherein the rhabdovirus is a VSV.

71. The method according to any one of claims 67 to 70, wherein the first cell population comprises HEK293T cells.

72. The method according to any one of claims 67 to 71, wherein the second cell population comprises BHK21 cells.

73. The method according to any one of claims 67 to 72, wherein the second cell population comprises Vero cells.

74. The method according to any one of claims 67 to 73, wherein the nucleocapsid, the phosphoprotein, or the large protein, or a combination thereof, are conditionally functional.

75. The method according to any one of claims 67 to 73, wherein the nucleocapsid, the phosphoprotein, or the large protein, or a combination thereof, are nonfunctional.


SEQUENCE LISTING (10 sequences)

SEQ ID NO: 1 - DNA, 4470 bp, synthetic construct
  source 1..4470: mol_type = other DNA
actgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgaccaccatgcacctgagaatccacgcgagacggagccctcctcgccggccggcctggacgcttgggatctggttcctgttctggggatgtatcgtcagctctgtatggagtcaattgtcttctaatgtagcttcctcctcctccacctcttcctcgccggggtctcactacccatacgatgtgccagattacgccaaccccacggagccgggaatcagacgggttccgggggcctcagaggtgatccgggagtcgagcagcacaacagggatggtcgtcggcattgtggctgctgccgccctctgcatcttgatcctcctgtacgccatgtacaagtacaggaacagggacgagggctcgggctcgacctcgggctcgggcagcggtggctctggaggttctggtggcagctctggaggtatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaaggcagcgaagtttgatacagagactaaggattgtaatgaggcggcgaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatagcggtagtggcggcggcagtgggggctctggtgggtcaggtggcagtggcggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaggatctagagggggatctggtggatcagggggtagcgggggttctggcggaccggcaggcagtagcggaagcagtattatacctgacagggaggttctctaccaggagttcgatgagatggaagagtgctctcagcacctccccggcgcgcctgctagctctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggattaacggcagtggagagggcagaggaagtctgctaacatgcggtgacgtcgaggagaatcctggcccaatgggcaagccactgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcatgttagctccctatatcccagagtatgtcgcctatgttgccaaagaccctgtgaatcagagagcctgccacattctggagtgtcccgaagggcttgcccaggatgtcatcagcaccattggccaggccttcgagttgcgcttcaaacaatacctcagggacatcgagcaggtgccccagcagcccacccttaagatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttctcggacaattcctccccaccagcggtgtgagggccgctcgagcctaagcttctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacctgcaggtcggagtactgtcctccgagcggagtactgtcctccgagcggagtactgtcctccgagcggagtactgtcctccgagcggagtactgtcctccgagcggagactctagcgagcgcgtactgccgccaccatgg

SEQ ID NO: 2 - protein, 721 aa, synthetic construct
MHLRIHARRSPPRRPAWTLGIWFLFWGCIVSSVWSQLSSNVASSSSTSSSPGSHYPYDVPDYANPTEPGIRRVPGASEVIRESSSTTGMVVGIVAAAALCILILLYAMYKYRNRDEGSGSTSGSGSGGSGGSGGSSGGMNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHSGSGGGSGGSGGSGGSGGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRGSRGGSGGSGGSGGSGGPAGSSGSSIIPDREVLYQEFDEMEECSQHLPGAPASSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSMGPKKKRKVAPPTDVSLGDELHLDGEDVAMAHADALDDFDLDMLGDGDSPGPGFTPHDSAPYGALDMADFEFEQMFTDALGIDEYGGIN

SEQ ID NO: 3 - protein, 400 aa, synthetic construct
MGKPLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGMLAPYIPEYVAYVAKDPVNQRACHILECPEGLAQDVISTIGQAFELRFKQYLRDIEQVPQQPTLKMDYKDDDDKGSSGTGSGSGTSAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFSDNSSPPAV

SEQ ID NO: 4 - DNA, 3581 bp, synthetic construct
  source 1..3581: mol_type = other DNA
actgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgccaccatgggcaagccactgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcatgttagctccctatatcccagagtatgtcgcctatgttgccaaagaccctgtgaatcagagagcctgccacattctggagtgtcccgaagggcttgcccaggatgtcatcagcaccattggccaggccttcgagttgcgcttcaaacaatacctcagggacatcgagcaggtgccccagcagcccacccttaagatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttctcggacaattcctccccaccagcggtgggctctggagccaccaacttcagcctgctgaagcaggcaggcgacgtggaagagaaccctggccctgctagctctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggcttaagggatccggtaaaccaatccccaatcctctgctgggtcttgatagcaccgatgagatggaagagtgctctcagcacctccccggcggttcgggaggttctggtgggtcaggtggcagtggcggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaggatctagagggggatctggtggatcagggggtagcgggggttctggcggacaattcaagatgagcaaggacggcaagaagaaaaagaagaagtccaagactaagtgcgtgatcatgtgagggccgctcgagcctaagcttctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacctgcaggtcggagtactgtcctccgagcggagtactgtcctccgagcggagtactgtcctccgagcggagtactgtcctccgagcggagtactgtcctccgagcggagactctagcgagcgcgtactgccgccaccatgg

SEQ ID NO: 5 - protein, 423 aa, synthetic construct
ASSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSMGPKKKRKVAPPTDVSLGDELHLDGEDVAMAHADALDDFDLDMLGDGDSPGPGFTPHDSAPYGALDMADFEFEQMFTDALGIDEYGGLKGSGKPIPNPLLGLDSTDEMEECSQHLPGGSGGSGGSGGSGGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRGSRGGSGGSGGSGGSGGQFKMSKDGKKKKKKSKTKCVIM

SEQ ID NO: 6 - DNA, 18594 bp, synthetic construct
  source 1..18594: mol_type = other DNA
accaaacaaagttgggtaaggatagttcaatcaatgatcatcttctagtgcacttaggattcaagatcctattatcagggacaagagcaggattagggatatcccgacgcgtacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagactgacgtctcgcgatcgcgtccgcctaccctccatcattgttataaaaaacttaggaaccaggtccacacagccgccagcccatcaaccatccactcccacgattggagatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgagaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggacatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcatcggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgaggaccagaacaacatccgcctaccctccatcattgttataaaaaacttaggaaccaggtccacacagccgccagcccatcaaccatccactcccacgattggagcagatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggctacagtgtcattatgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtcttcattgacaggtggtgcaacccaatgtgctcgaaagtcaccctcggaaccatcagggccaggtgcacctgcggggaacgtccccgagtgtgtgagcaatgccgcactgatacaggagtggacacccgaatctggcaccacaatctccccgagatcccagaataatgaagaagggggagaccattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaaggatgagatggaagagtgctctaagcacctcggcggttcgggaggttctggtgggtcaggtggcagtggcaagccaatggcgggcaacttcgactcggaggagcggagtagctggtactgggggcggttgagtcggcaggaggcggtggcgctgctgcagggccagcggcacggggtgttcctggtgcgggactcgagcaccagccccggggactatgtgctcagcgtctcagagaactcgcgcgtctcccactacatcatcaacagcagcggcccgcgcccgccggtgccaccgtcgcccgcccagcctccgcccggggtgagcccctccagactccgaataggagatcaagagtttgattcattgcctgctttactggaattctacaaaatacactatttggacactacaacgttgatagaaccagttcttaagggatctagaggtaaaccaatccccaatcctctgctgggtcttgatagcaccgaattcaagatgagcaaggacggcaagaagaaaaagaagaagtccaagactaagtgcgtgatcatgtgatgcgcatctacagctcaacttacctgccaaccccatgccagtcgacccaactagtacaacctaaatccattataaaaaacttaggagcaaagtgattgcctcccaagttccacattcgaaaccatgggcaagccactgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcggggatccggacacagccgagtatgtcgcctatgttgccaaagaccctgtgaatcagagagcctgccacattctggagtgtcccgaagggcttgcccaggatgtcatcagcaccattggccaggccttcgagttgcgcttcaaacaatacctcagggacatcgagcaggtgccccagcagcccacccttaagatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatagggcgcgccttgactacagctcaacttacctgccaaccccatgccagtcgacccaactagtacaacctaaatccattataaaaaacttaggagcaaagtgattgcctcccaagttccacaatgacagagatctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggtccctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctggaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcagtgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggtttccaagcggaagaacaatatcggtagttaattaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaaccaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaattgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgatgggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaggtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaaggggggtttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcaaggtctaagaccggtagagaaatgtgcagttctaaccgaccatatcaaggcagaggctatgttatctccagcaggatcttcgtggaacataaatccaattattgtagaccattactcatgctccctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaggagatacttaaactaagcaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatatctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattttgggggcacattcttctttactccgggaacagaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 7 - protein, 691 aa, synthetic construct
MAEEQARHVKNGLECIRALKAEPIGSLAIEEAMAAWSEISDNPGQERATCREEKAGSSGLSKPCLSAIGSTEGGAPRIRGQGPGESDDDAETLGIPPRNLQASSTGLQCHYVYDHSGEAVKGIQDADSIMVQSGLDGDSTLSGGDNESENSDVDIGEPDTEGYAITDRGSAPISMGFRASDVETAEGGEIHELLRLQSRGNNFPKLGKTLNVPPPPDPGRASTSGTPIKKGTDARLASFGTEIASSLTGGATQCARKSPSEPSGPGAPAGNVPECVSNAALIQEWTPESGTTISPRSQNNEEGGDHYDDELFSDVQDIKTALAKIHEDNQKIISKLESLLLLKGEVESIKKQINRQNISISTLEGHLSSIMIAIPGLGKDPNDPTADVEINPDLKPIIGRDSGRALAEVLKKPVASRQLQGMTNGRTSSRGQLLKEFQLKPIGKKMSSAVGFVPDTGPASRSVIRSIIKSSRLEEDRKRYLMTLLDDIKGANDLAKFHQMLMKIIMKEDVVCCSMSYGGSGGSGGSGGSGKPMAGNFDSEERSSWYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPVLKGSRGKPIPNPLLGLDSTEFKMSKDGKKKKKKSKTKCVIM

SEQ ID NO: 8 - protein, 386 aa, synthetic construct
MGKPLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRDIEQVPQQPTLKMDYKDDDDKGSSGTGSGSGTSAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMR

SEQ ID NO: 9 - DNA, 17025 bp, synthetic construct
  source 1..17025: mol_type = other DNA
acgaagacaaacaaaccattattatcattaaaaggctcaggagaaactttaacagtaatcagaattctcgaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttaatctgcaccaccggcaagctgcccgtgccctggcccaccctcgtgaccaccttaggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccctcctcaggtgctggccatatgaaaaaaactaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatgggcaagccactgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcatgttagctccctatatcccagagtatgtcgcctatgttgccaaagaccctgtgaatcagagagcctgccacattctggagtgtcccgaagggcttgcccaggatgtcatcagcaccattggccaggccttcgagttgcgcttcaaacaatacctcagggacatcgagcaggtgccccagcagcccacccttaagatggattacaaggatgacgacgataagggctcttccgggacaggctccggatccggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagagacaaccatgagatccccggtgttctcggacaattcctccccaccagcggtgggctctggagccaccaacttcagcctgctgaagcaggcaggcgacgtggaagagaaccctggccctatggataatctcacaaaagttcgtgagtatctcaagtcctactctcgtctagatcaggcggtaggagagatagatgagatcgaagcacaacgagctgaaaagtccaattatgagttgttccaagaggacggagtggaagagcatactaggccctcttattttcaggcagcagatgattctgacacagaatctgaaccagaaattgaagacaatcaaggcttgtatgtaccagatccggaagctgagcaagttgaaggctttatacaggggcctttagatgactatgcagatgaggacgtggatgttgtattcacttcggactggaaacagcctgagcttgaatccgacgagcatggaaagaccttacggttgacattgccagagggtttaagtggagagcagaaatcccagtggcttttgacgattaaagcagtcgttcaaagtgccaaacactggaatctggcagagtgcacatttgaagcatcgggagaaggggtcatcataaaaaagcgccagataactccggatgtatataaggtcactccagtgatgaacacacatccgtaccaatcagaagccgtatcagatgtttggtctctctcaaagacatccatgactttccaacccaagaaagcaagtcttcagcctctcaccatatccttggatgaattgttctcatctagaggagaattcatctctgtcggaggtaacggacgaatgtctcataaagaggccatcctgctcggtctgaggtacaaaaagttgtacaatcaggcgagagtcaaatattctctgggtaaaccaatccccaatcctctgctgggtcttgatagcaccgatgagatggaagagtgctctcagcacctccccgggggtagcgggggttctggcggttcgggaggttctaagcctttttctacacaagacaccgacctcgacttggaaatgctcgctccatatatccctatggaggaggaatttcaactcagatcatttgaccagctctccccaaaaaagaaaggcggttcgggaggttctggtgggtcaggtggcagtggcggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaggatctagagggggatctggtggatcagggggtagcgggggttctggcggacaattcaagatgagcaaggacggcaagaagaaaaagaagaagtccaagactaagtgcgtgatcatgtgaactatgaaaaaaagtaacagatatcacaatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctagcttccagcttctgaacaatccccggtttactcagtctctcctaattccagcctttcgaacaactaatatcctgtcttttctatccctatgaaaaaaactaacagagatcgatctgtttccttgacacgcgtcacgatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcgcggccgccctgcacaacagattcttcatgtttgaaccaaatcaacttgtgatatcatgctcaaagaggccttaattatattttaatttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttggcagttatccagcaatcatttccgattaggtgggaggctatttcagtaaaaggaggatacaagcagaagtggagtactagaggtgatgggctcccaaaagatacccgaatttcagactccttggccccaatcgggaactggatcagatctttggaattggtccgaaaccaagttcgtctaaatccattcaataagatcttgttcaatcagctatgtcgtacagtggataatcatttgaagtggtcaaatttgcgaaaaaacacaggaatgattgaatggatcaatgggcgaatttcaaaagaagaccggtctatactgatgttgaagagtgacctacatgaggaaaactcttggagagattaaaaaatcaggaggagactccaaactttaagtatgaaaaaaactttgatccttaagaccctcttgtggtttttatttttttatctggttttgtggtcttcgtgggtcggcatggcatctccacctcctcgcggtccgacctgggcatccgaaggaggacgtcgtccactcggatggctaagggagagctcggatccggctgctaacaaagcccgaaaggaagctgagttggctgctgccaccgctgagcaataactagcataaccccttggggcctctaaacgggtcttgaggggttttttgctgaaaggaggaactatatccggatcgagatcctctagagtcgacctgcaggcatgcaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggggatctcgatcccgcgaaattaatacgactcactatagg

SEQ ID NO: 10 - protein, 503 aa, synthetic construct
MDNLTKVREYLKSYSRLDQAVGEIDEIEAQRAEKSNYELFQEDGVEEHTRPSYFQAADDSDTESEPEIEDNQGLYVPDPEAEQVEGFIQGPLDDYADEDVDVVFTSDWKQPELESDEHGKTLRLTLPEGLSGEQKSQWLLTIKAVVQSAKHWNLAECTFEASGEGVIIKKRQITPDVYKVTPVMNTHPYQSEAVSDVWSLSKTSMTFQPKKASLQPLTISLDELFSSRGEFISVGGNGRMSHKEAILLGLRYKKLYNQARVKYSLGKPIPNPLLGLDSTDEMEECSQHLPGGSGGSGGSGGSKPFSTQDTDLDLEMLAPYIPMEEEFQLRSFDQLSPKKKGGSGGSGGSGGSGGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRGSRGGSGGSGGSGGSGGQFKMSKDGKKKKKKSKTKCVIM